(19) United States
(12) Patent Application Publication
(10) Pub. No.: US 2019/0048342 A1
(43) Pub. Date: Feb. 14, 2019

(54) CROSS-REGULATION OF TYPE I INTERFERON SIGNALING PATHWAYS

(71) Applicant: THE METHODIST HOSPITAL SYSTEM, Houston, TX (US)

(72) Inventors: Rongfu WANG, Houston, TX (US); Yicheng WANG, Houston, TX (US); Xiao YU, Houston, TX (US)

(73) Assignee: The Methodist Hospital System, Houston, TX (US)

(21) Appl. No.: 16/075,661

(22) PCT Filed: Feb. 3, 2017
(86) PCT No.: PCT/US2017/016450

§ 371 Date: Aug. 5, 2018

Related U.S. Application Data
(60) Provisional application No. 62/292,188, filed on Feb. 5, 2016.

(51) Int. Cl. C12N 15/113; C07K 14/705; A61K 31/713; A61K 38/17; A61K 35/68; A61K 38/21; A61K 39/015; A61K 39/00; A61K 45/06; A61K 38/20; A61P 35/00; A61P 33/06

(52) U.S. Cl. CPC C12N 15/113; C12N 2320/31; A61K 31/713; A61K 38/177; A61K 35/68; A61K 38/215; A61K 38/212; A61K 39/015; A61K 39/0011; A61K 45/06; A61K 38/20; A61P 35/00; A61P 33/06; C12N 2310/14; C07K 14/70596

(57) ABSTRACT

Compositions and methods for cross-regulation of type I interferon signaling pathways in pDCs for vaccine development are provided.

### CROSS-REFERENCE TO RELATED APPLICATIONS

This application claims benefit of U.S. Provisional Application No. 62/292,188, filed Feb. 5, 2016, which is hereby incorporated herein by reference in its entirety.

### STATEMENT REGARDING FEDERALLY SPONSORED RESEARCH OR DEVELOPMENT

This invention was made with Government Support under Grant Nos. R01CA090327 and RO1CA101795 awarded by the National Institutes of Health. The Government has certain rights in the invention.

### BACKGROUND

The development of a malaria vaccine has faced several obstacles: the lack of a traditional market, few developers, and the technical complexity of developing any vaccine against a parasite. Malaria parasites have a complex life cycle, and there is poor understanding of the complex immune response to malaria infection. Malaria parasites are also genetically complex, producing thousands of potential antigens. Unlike the diseases for which we currently have effective vaccines, exposure to malaria parasites does not confer lifelong protection. Acquired immunity only partially protects against future disease, and malaria infection can persist for months without symptoms of disease.

### SUMMARY

Identified herein is a previously unrecognized regulatory mechanism between different type I IFN signaling pathways in pDCs, providing new strategies for development of therapeutic vaccines against infectious diseases and cancer. For example, a composition is disclosed that contains a TLR9 or TLR7 ligand, e.g., CpG oligonucleotide, and a SOCS1 pathway antagonist in a pharmaceutically acceptable carrier, which enhance effectiveness of an antigen or pathogen for vaccination. Therefore, in some embodiments, the composition further comprises a pathogen, such as a Plasmodium, e.g., live, attenuated or dead.

The SOCS1 pathway antagonist can be direct or indirect. In some embodiments, the SOCS1 pathway antagonist is a direct SOCS1 inhibitor. For example, the SOCS1 inhibitor can be a gene silencing functional nucleic acid, e.g., RNAi or siRNA. In some embodiments, the SOCS1 pathway antagonist comprises a TBK1 inhibitor or an IRF3 inhibitor.

In some embodiments, the composition further contains interferon-α, interferon-β, or a combination thereof. In some embodiments, the composition further contains an adjuvant.

Also disclosed is a method for vaccinating a subject for a pathogen by administering to a subject in need thereof a composition disclosed herein.

Also disclosed is a method for vaccinating a subject for cancer that involves administering to a subject in need thereof a composition disclosed herein in combination with a tumor antigen. The tumor antigen can be administered separately, but in some embodiments, the composition contains the tumor antigen. In some embodiments, the method further comprises administering to the subject a composition comprising a checkpoint inhibitor. In some embodiments, the method further comprises administering to the subject a composition comprising interleukin-2, interferon-α, or combination thereof.

Also disclosed is a method for enhancing tumor immunity in a subject that involves administering to a subject diagnosed with a tumor a composition disclosed herein. In some embodiments, the method further comprises administering to the subject a composition comprising a tumor antigen. In some embodiments, the method further comprises administering to the subject a composition comprising a checkpoint inhibitor. In some embodiments, the method further comprises administering to the subject a composition comprising interleukin-2, interferon-α, or combination thereof.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A to 1I show that mice deficient in cGAS, Sting, Mda5, Mavs, or Irf3, but not in TLR signaling molecules, are protected from P. yoelii YM infection. FIGS. 1A to 1C show daily YM parasitemias and mortality rates of WT (squares) and TLR deficient mice (circles) after Plasmodium yoelii YM (0.5×10⁶ iRBCs):Tlr7^{-/-} (FIG. 1A), Tlr9^{-/-} (FIG. 1B) and Myd88^{-/-} (FIG. 1C). FIGS. 1D to 1I show daily YM parasitemias and mortality rates of WT (squares) and cGAS^{-/-}, Sting^{-/-}, Mda5^{-/-}, Mavs^{-/-}, Irf3^{-/-}, and Irf7^{-/-} mice (circles) after Plasmodium yoelii YM (1×10⁶ iRBCs): cGAS^{-/-} (FIG. 1D), Sting^{-/-} (FIG. 1E), Mda5^{-/-} (FIG. 1F), Mavs^{-/-} (FIG. 1G), Irf3^{-/-} (FIG. 1H), and Irf7^{-/-} (FIG. 1I) mice. Data are representative of three independent experiments and are plotted as the mean±s.d. *P<0.05, **P<0.01, ***P<0.001 vs. corresponding control. NS, not significant.

FIGS. 2A to 2H show DNA and RNA sensors of P. yoelii YM infection and type I IFN production in cells and in serum of mice deficient in Mda5, Mavs, Sting, or cGAS. FIG. 2A is a schematic representation of type I IFN signaling pathways leading to the induction of IFN-3 transcription after in vitro stimulation with gDNA or RNA of P. yoelii YM. FIG. 2B shows quantitative analysis of IFN-β mRNA in RAW264.7 cells stimulated with purified P. yoelii YM gDNA or RNA from YM-infected RBCs. gDNA or RNA purified from uninfected RBCs served as a control. FIG. 2C shows quantitative analysis of IFN-β mRNA in BMDMs derived from WT and deficient (Mda5^{-/-}, Mavs^{-/-}, and Sting^{-/-}) mice after P. yoelii YM gDNA stimulation.

FIG. 2D shows quantitative analysis of IFN-β mRNA in cDCs derived from WT and deficient (Mda5^{-/-}, Mavs^{-/-}, and Sting^{-/-}) mice after P. yoelii YM gDNA or RNA stimulation. FIG. 2E shows quantitative analysis of IFN-β mRNA in STING stably expressing 293T cells transfected with Flag-cGAS, Dai, Ddx41, or Ifi16 followed by P. yoelii YM DNA stimulation for 18 h. FIG. 2F shows quantitative analysis of IFN-β mRNA in RAW264.7 cells transfected with cGAS-specific siRNA, Dai-specific siRNA, Ddx41-specific siRNA, Ifi16-specific siRNA or scramble siRNA for 48 h, followed by P. yoelii YM gDNA stimulation for 6 h. FIG. 2G shows ELISA analysis of cell supernatants from RAW264.7 cells transfected with cGAS-specific siRNA or scramble siRNA, followed by P. yoelii YM DNA stimulation for 24 h. FIG. 2H shows serum levels of IFN-α and IFN-β from WT and KO (Mda5^{-/-}, Mavs^{-/-}, Sting^{-/-} and cGAS^{-/-}) mice (n=5) infected with P. yoelii YM. Data are plotted as the mean±s.d. and are representative of three independent experiments. *P<0.05, **P<0.01, ***P<0.001 vs. corresponding control. ND, not detected; NS, not significant.

FIGS. 3A to 3F shows the requirement and regulation of TLR7-MyD88-IRF7 in type I IFN-mediated protection of mice from P. yoelii YM infection. FIG. 3A shows serum levels of IFN-α and IFN-β in WT, Myd88^{-/-}, Mavs^{-/-}, and Mavs^{-/-}:Myd88^{-/-} mice at 0, 18, and 24 h after P. yoelii YM infection. FIG. 3B shows daily YM parasitemias and mortality rates of WT, Myd88^{-/-}, Mavs^{-/-}, and Mavs^{-/-}:Myd88^{-/-} mice after P. yoelii YM (1×10⁶ iRBCs) infection. FIG. 3C shows serum levels of IFN-α and IFN-β in WT, Tlr7^{-/-}, Sting^{-/-}, and Sting^{-/-}:Tlr7^{-/-} mice at 0, 24, and 48 h after P. yoelii YM infection. FIG. 3D shows serum levels of IFN-α and IFN-β in WT and KO (Irf3^{-/-}, Irf7^{-/-}, and Irf3^{-/-}:Irf7^{-/-}) at 24 h after P. yoelii YM infection. FIG. 3E shows daily YM parasitemias and mortality rates of WT and KO (Tlr7^{-/-}, Sting^{-/-}, and Sting^{-/-}:Tlr7^{-/-}) mice after P. yoelii YM (1×10⁶ iRBCs) infection. FIG. 3F shows daily YM parasitemias and mortality rates of WT and KO (Irf3^{-/-}, Irf7^{-/-}, and Irf3^{-/-}:Irf7^{-/-}) mice after P. yoelii YM (1×10⁶ iRBCs) infection. Data are plotted as the mean±s.d. and are representative of three independent experiments. **P<0.01, ***P<0.001 vs. corresponding control. ND, not detected.

FIGS. 4A to 3E show requirement of plasmacytoid DCs in type I IFN mediated protection of mice from P. yoelii YM infection. FIGS. 4A to 4C show depletion of pDCs cell population in WT, Mda5^{-/-}, Mavs^{-/-}, and Sting^{-/-} mice by administration of anti-mPDCA-1 antibody at 12 h before and 12 h after YM infection, rat IgG2b treatment served as a control. Serum levels of IFN-α and IFN-β collected at 24 h after infection in WT, Mda5^{-/-}, Mavs^{-/-}, and Sting^{-/-} mice untreated or treated with anti-mPDCA-1 are shown in FIG. 4A. Daily YM parasitemias and mortality rates of WT, Mda5^{-/-}, Mavs^{-/-}, and Sting^{-/-} mice untreated or treated with anti-mPDCA-1 are shown in FIGS. 4B and 4C. FIGS. 4D and 4E show WT, WT BDCA2-DTR, Mavs^{-/-}, and Mavs^{-/-}:BDCA2-DTR mice treated with DT (5 ng per gram body weight) as indicated at 1 day before and 1, 3, and 5 days after P. yoelii YM infection. Serum levels of IFN-α and IFN-β collected at 24 h after infection are shown in FIG. 4D. Daily YM parasitemias and mortality rates are shown in FIG. 4E. Data are plotted as the mean±s.d. and are representative of three independent experiments. *P<0.05, **P<0.01, ***P<0.001 vs. corresponding control. NS, not significant.

FIGS. 5A to 5I show SOCS1 is a key negative regulator induced by STING/MAVS-mediated type I signaling and inhibits MyD88-dependent type I IFN signaling in pDCs. FIG. 5A shows expression of negative regulators Socs1 in the spleens of WT and deficient (Mda5^{-/-}, Mavs^{-/-}, and Sting^{-/-}) mice at the indicated time points after P. yoelii YM infection. RNAs from splenocytes were isolated and used for expression analysis. FIGS. 5B and 5C show expression of Socs1 in pDCs of WT and KO (Mda5^{-/-}, Mavs^{-/-}, and Sting^{-/-}) mice after P. yoelii YM RNA (FIG. 5B) or gDNA (FIG. 5C) stimulation. FIG. 5D shows WT and KO (Mavs^{-/-}) mice infected with P. yoelii YM for 18 h. PDCs, cDCs, and macrophages were isolated and purified from infected mice by cell isolation kits, and then used for analysis of Socs1 mRNA expression. FIG. 5E shows IFN-α and IFN-β expression at the mRNA and protein levels in pDCs, cDCs or macrophages from WT and Mavs^{-/-} mice after overnight culture. FIG. 5F shows SOCS1 interacts with MyD88 and IRAK1. 293T cells were transfected with HA-Socs1 and Flag-Irak1, Flag-Irak4, or Flag-Myd88. Immunoprecipitation of cell lysates was performed with an anti-Flag, followed by immunoblotting with an anti-HA or anti-Flag antibody. Whole cell lysates were immunoblotted with either an anti-HA or anti-Flag antibody for protein expression. Anti-β-actin blot was served as a control. FIGS. 5G to 5I show C57BL/6 WT mice treated with Socs1-specific or scramble siRNA for 48 h, followed by P. yoelii YM (0.5×10⁶ iRBCs) infection. Diagram of experiment procedure are shown in FIG. 5G. Serum levels of IFN-α, IFN-β, and IFN-γ in Socs1-specific or scrambled siRNA-treated mice at 24 h after P. yoelii YM infection are shown in FIG. 5H. Daily YM parasitemias and mortality rates of Socs1-specific or scrambled siRNA-treated mice after P. yoelii YM infection are shown in FIG. 5I. Data are plotted as the mean±s.d. and are representative of three independent experiments. **P<0.01, ***P<0.001 vs. corresponding control. NS, not significant. ND, not detected.

FIGS. 6A to 6H show SOCS1 is responsible for inhibition of Myd88-dependent type I IFN in pDCs and finely tuned mice resistance. FIGS. 6A and 6B show WT mice treated with scramble siRNA, Socs1-specific siRNA, or Socs1-specific siRNA with anti-mPDCA-1 antibody at 24 h before infection, followed by P. yoelii YM (0.5×10⁶ iRBCs) infection. Serum levels of IFN-α and IFN-β collected at 24 h after infection are shown in FIG. 6A. Daily YM parasitemias and mortality rates are shown in FIG. 6B. FIG. 6C shows WT and Myd88¹ mice treated with Socs1-specific or scramble siRNA for 48 h, followed by P. yoelii YM (0.5×10⁶ iRBCs) infection. Daily YM parasitemias and mortality rates of Socs1-specific or scramble siRNA treated WT and Myd88^{-/-} mice after P. yoelii YM infection were monitored. FIG. 6D shows intracellular staining for Socs1, IFN-α, and IFN-β of uninfected or infected WT and KO mice (Sting^{-/-}, Mda5^{-/-}, and Mavs^{-/-} mice) splenocytes at 18 h post P. yoelii YM infection and gated for pDCs using CD11b⁻CD11c⁺B220⁺. FIGS. 6E to 6G show WT and KO (Sting^{-/-}, Mda5^{-/-}, Mavs^{-/-}, Mavs^{-/-}:Sting^{-/-}, and Irf3^{-/-}) mice infected with high dose of P. yoelii YM (1×10⁷ iRBCs). Serum levels of IFN-α and IFN-β in WT and KO (Sting^{-/-}, Mda5^{-/-}, Mavs^{-/-}, Mavs^{-/-}:Sting^{-/-}, and Irf3^{-/-}) at 24 h after P. yoelii YM infection are shown in FIG. 6E. YM parasitemias of WT and KO (Sting^{-/-}, Mda5^{-/-}, Mavs^{-/-}, Mavs^{-/-}:Sting^{-/-} and Irf3^{-/-}) mice at day 5 after P. yoelii YM infection are shown in FIG. 6F, and daily YM parasitemias over 30 days and mortality rates of WT and KO (Sting^{-/-}, Mda5^{-/-}, Mavs^{-/-}, Mavs^{-/-}:Sting^{-/-} and Irf3^{-/-}) mice after P. yoelii YM infection are shown in FIG. 6G. Data are plotted as the mean±s.d. and are representative of three independent experiments. *P<0.05, **P<0.01, ***P<0.001 vs. corresponding control. NS, not significant. FIG. 6H shows a working model to illustrate how DNA/RNA sensors (cGAS, MDA5 and TLR7) detect malaria infection and activate two type I IFN signaling pathways in pDCs. Importantly, cGAS-STING and MDA5-MAVS-induced IRF3-dependent type I IFN signaling inhibits TLR7-MyD88-induced IRF7-dependent type IFN signaling pathway through upregulation of SOCS1 in WT pDCs in response to lethal P. yoelii YM infection. Deficiency in cGAS, STING, MDA5, MAVS or IRF3 markedly increases IFN-α and IFN-β production at the early stage of infection (24 h) in KO pDCs in response to lethal P. yoelii YM infection.

FIGS. 7A to 7G show Plasmodium. yoelii YM infection inhibits melanoma growth by activating type I IFN signaling. FIG. 7A shows serum cytokine levels of IFN-α and IFN-β in WT mice at 24 h after indicated irradiation doses for YM. FIG. 7B shows images of spleens and lungs from WT mice at 15 days post YM infection as indicated. FIG. 7C shows spleen sizes and weights of lungs from WT mice at 15 days post YM infection as indicated. FIG. 7D shows experimental design and procedures for a B16 melanoma tumor model to assess antitumor immunity induced by DC/TRP-2 vaccines. FIG. 7E shows serum cytokine levels of IFN-α and IFN-β in WT mice (day 5) at 24 h after malaria infection. FIG. 7F shows pathological images of B16 lung metastasis at 15 days after DC/TRP-2 vaccine with or without irradiated YM infection as indicated. FIG. 7G shows numbers of B16 lung metastasis in WT mice at 15 days after DC/TRP-2 vaccine with or without irradiated YM infection. Data are plotted as the mean±s.d. and are representative of three independent experiments. *P<0.05, **P<0.01, ***P<0.001 vs. corresponding control. NS, not significant.

FIGS. 8A to 8C show Mavs deficiency increases type I IFN signaling and antitumor immunity. FIG. 8A shows serum cytokine levels of IFN-α and IFN-β in WT and Mavs^{-/-} mice treated with DC/TRP-2 peptide or control peptide, with or without irradiated YM infection. Serum were collected at 24 h after YM infection and used for measuring type I IFN cytokines by ELISA. FIG. 8B shows pathological images of B16 lung metastasis in WT and Mavs^{-/-} mice at 15 days after DC/TRP-2 vaccine with or without irradiated YM infection. FIG. 8C shows numbers of B16 lung metastasis in WT and Mavs^{-/-} mice at 15 days after DC/TRP-2 vaccine with or without irradiated YM infection. Data are plotted as the mean±s.d. and are representative of three independent experiments. *P<0.05, **P<0.01, ***P<0.001 vs. corresponding control. NS, not significant.

FIGS. 9A to 9E show Tlr2^{-/-}, Tlr3^{-/-}, Tlr4^{-/-}, Trif^{-/-}, and Rig-i^{-/-} mice are susceptible to Plasmodium yoelii YM infection. Daily YM parasitemias and mortality rates of WT (circles) and deficient mice (squares). C57BL/6 (WT), Tlr2^{-/-}, Tlr3^{-/-}, Tlr4^{-/-}, Trif^{-/-}, and Rig-i^{-/-} mice were intraperitoneally infected with P. yoelii YM (0.2-0.5×10⁶ iRBCs). Tlr2^{-/-} (FIG. 9A), Tlr3^{-/-} (FIG. 9B), Tlr4^{-/-} (FIG. 9C), Trif^{-/-} (FIG. 9D), and Rig-i^{-/-} (FIG. 9E). Data are plotted as the mean±s.d. and representative of three independent experiments. NS, not significant.

FIGS. 10A to 10G show type I IFN gene expression after P. yoelii YM gDNA or RNA stimulation and WT mice serum cytokine level after P. yoelii YM infection. FIG. 10A shows quantitative analysis of IFN-β mRNA in RAW264.7 cells 6 h after stimulation with gDNA or RNA of P. yoelii YM, without or with DNase or RNase pretreatment. FIG. 10B shows ELISA analysis of cell supernatants from BMDMs derived from WT and deficient (Mda5^{-/-}, Mavs^{-/-}, and Sting^{-/-}) mice stimulated with P. yoelii YM gDNA for 24 h. FIG. 10C shows ELISA analysis of cell supernatants from cDCs derived from WT and deficient (Mda5^{-/-}, Mavs^{-/-}, and Sting^{-/-}) mice stimulated with P. yoelii YM gDNA or RNA for 24 h. FIG. 10D shows quantitative analysis of IFN-β mRNA in RAW264.7 cells transfected with RNA pol III-specific siRNA or scramble siRNA for 48 h, followed by P. yoelii YM gDNA stimulation for 6 h. FIG. 10E shows quantitative analysis of IFN-β mRNA in RAW264.7 cells pretreated with ML60218 or DMSO for 24 h, followed by P. yoelii YM gDNA stimulation for 6 h. FIG. 10F shows knockdown efficiency of cGAS, Dai, Ddx41, and Ifi16 in RAW264.7 cells transfected with specific siRNAs for cGAS, Dai, Ddx41, Ifi16 or scrambled siRNAs for 48 h. FIG. 10G shows serum levels of IFN-α and IFN-β from WT mice at the indicated times after P. yoelii YM infection, assessed by ELISA. Data are plotted as the mean±s.d. and are representative of three independent experiments. *P<0.05, **P<0.01, ***P<0.001 vs. corresponding control. ND, not detected; NS, not significant.

FIGS. 11A to 11H show the requirement and regulation of TLR7-MyD88-IRF7 in type I IFN mediated protection of mice from P. yoelii YM infection. FIGS. 11A and 11B show serum levels of IFN-α and IFN-β in WT, Myd88^{-/-}, Mda5^{-/-}, Mda5^{-/-}:Myd88^{-/-}, Sting^{-/-} and Sting^{-/-}:Myd88^{-/-} mice at 0, 18, and 24 h after P. yoelii YM infection. FIGS. 11C and 11D show daily YM parasitemias and mortality rates of WT, Myd88^{-/-}, Mda5^{-/-}, Mda5^{-/-}:Myd88^{-/-}, Sting^{-/-} and Sting^{-/-}:Myd88^{-/-} mice after P. yoelii YM (1×10⁶ iRBCs) infection. FIG. 11E shows serum levels of IFN-α and IFN-β in WT, Tlr7, Mavs^{-/-}, Mavs^{-/-}:Tlr7^{-/-}, Mda5^{-/-} and Mda5^{-/-}:Tlr7^{-/-} mice at 24 h after P. yoelii YM infection. FIG. 11F shows daily YM parasitemias and mortality rates of WT and KO (Tlr7^{-/-}, Mavs^{-/-}, Mavs^{-/-}:Tlr7^{-/-}, Mda5^{-/-} and Mda5^{-/-}:Tlr7^{-/-}) mice after P. yoelii YM (1×10⁶ iRBCs) infection. FIG. 11G shows serum levels of IFN-α and IFN-β in WT, Tlr9^{-/-}, Mavs^{-/-}, Mavs^{-/-}:Tlr9^{-/-}, Mda5^{-/-} and Mda5^{-/-}:Tlr9^{-/-} mice at 24 h after P. yoelii YM infection. FIG. 11H shows daily YM parasitemias and mortality rates of WT and KO (Tlr9^{-/-}, Mavs^{-/-}, Mavs^{-/-}:Tlr9^{-/-}, Mda5^{-/-} and Mda5^{-/-}:Tlr9^{-/-} mice) mice after P. yoelii YM (1×10⁶ iRBCs) infection. Data are plotted as the mean±s.d. and are representative of three independent experiments. **P<0.01, ***P<0.001 vs. corresponding control. ND, not detected. NS, not significant.

FIG. 12 shows detection of malaria 18S rRNA in pDC, cDC and macrophage. The cell populations of pDC, cDC and macrophage were isolated from WT mice splenocytes at 18 h post YM infection by cell isolation kits, and then analyzed for cell-specific expression of P. yoelii 18S rRNA by PCR. Data are representative of three independent experiments.

FIGS. 13A to 13E show expression of putative negative regulators of type I interferon signaling after P. yoelii YM infection. FIG. 13A shows expression of putative negative regulators Socs3, Ship1, Ship2, Duba, Gsk3β, Nlrc3, Pcbp2, Raul, and Rnf5 in the spleens of WT and deficient (Mda5^{-/-}, Mavs^{-/-}, and Sting^{-/-}) mice at the indicated times after P. yoelii YM infection. RNA from splenocytes was isolated and used for expression analysis using qPCR. FIG. 13B shows purification of pDCs (CD11b⁻B220⁺CD11c⁺) by FACS analysis. The last panel shows the percentage of pDCs after sorting. FIG. 13C shows expression of Socs3, Ship1, and Ship2 in pDCs of WT and deficient (Mda5^{-/-}, Mavs^{-/-}, and Sting^{-/-}) mice after P. yoelii YM RNA or gDNA stimulation. FIG. 13D shows quantitative analysis of Socs1 mRNA, IFN-α and IFN-β mRNA in pDCs, cDCs, and macrophages from WT and deficient (Mda5^{-/-}, Mavs^{-/-}, and Sting^{-/-}) mice after co-culture with P. yoelii YM iRBCs for 9 h. FIG. 13E shows ELISA analysis of IFN-α and IFN-β protein in supernatants of pDCs, cDCs, and macrophages from WT and deficient (Mda5^{-/-}, Mavs^{-/-}, and Sting^{-/-}) mice after co-culture with P. yoelii YM iRBCs for 24 h. Data are plotted as the mean±s.d. and are representative of three independent experiments. *P<0.05, **P<0.01, ***P<0.001 vs. corresponding control. NS, not significant.

FIGS. 14A to 14I show SOCS1 is responsible for inhibition of Myd88 dependent type I IFN in pDCs. FIG. 14A shows knockdown efficiency of Socs1, Socs3, Ship1, and Ship2 in pDCs transfected with specific siRNAs for Socs1, Socs3, Ship1, Ship2, or scrambled siRNAs for 48 h. FIG. 14B shows expression of IFN-α and IFN-β mRNA in WT pDCs transfected with Socs1-specific, Socs3-specific, Ship1-specific, Ship2-specific, or scrambled siRNAs for 48 h, followed by P. yoelii YM RNA stimulation for 6 h. FIG. 14C shows IFN-β protein of pDCs transfected with Socs1-specific or scrambled siRNAs, followed by P. yoelii YM RNA stimulation for 24 h. FIG. 14D shows experimental procedure of knockout of Socs1 using CRISPR/Cas9 system in bone marrow for generating chimera mice. FIG. 14E shows Western blot analysis of Socs1 knockout in bone marrow cells after transduction with Socs1-specific or scrambled sgRNA lentiviral supernatant for 48 h. FIG. 14F shows serum levels of IFN-α, IFN-β, and IFN-γ in Socs1-specific or scramble sgRNA-treated mice at 24 h after P. yoelii YM infection. FIG. 14G shows daily YM parasitemias and mortality rates of Socs1-specific or scrambled sgRNA-treated mice after P. yoelii YM infection. FIG. 14H shows a working model to explain SOCS1 inhibition of Myd88 dependent type I IFN signaling and Jak1 dependent downstream signaling of type I IFN. FIG. 14I shows serum levels of IFN-α, IFN-β, and IFN-γ in Socs1-specific or scramble siRNA-treated Myd88^{-/-} mice at 24 h after P. yoelii YM infection. Data are plotted as the mean±s.d. and are representative of three independent experiments. **P<0.01, ***P<0.001 vs. corresponding control. NS, not significant.

FIGS. 15A and 15B show serum levels of IFN-α, IFN-β, IL-6, and IFN-γ in WT, Mda5^{-/-}, Mavs^{-/-}, and Sting^{-/-} mice in response to P. yoelii N67C (FIG. 15A) or P. yoelii N67 (FIG. 15B) infection. Data are plotted as the mean±s.d. and are representative of three independent experiments. **P<0.01, ***P<0.001 vs. corresponding control.

FIGS. 16A to 16F show type I IFN, blockade of its receptor (IFNAR) and downstream signaling determine the fate of P. yoelii YM-infected mice. FIG. 16A shows parasitemias and mortality rates of WT mice infected with P. yoelii YM, followed by intravenous administration of recombinant mouse IFN-α and IFN-β together, IL-6, IFN-γ, or control BSA protein at 18 h post infection. FIG. 16B shows parasitemias and mortality rates of WT mice infected with P. yoelii YM, followed by intravenous administration of recombinant mouse IFN-α and IFN-β at 18, 32, or 48 h post infection. FIGS. 16C and 16D show parasitemias (FIG. 16C) and mortality rates (FIG. 16D) of WT, Mda5^{-/-}, Mavs^{-/-}, and Sting^{-/-} mice treated with anti-IFNAR1 blocking antibody at days −1, 2, 4, and 6 (500 μg), followed by infection with P. yoelii YM (1-2×10⁶ iRBCs). FIGS. 16E and 16F show WT mice were treated with scrambled siRNA, Stat1-specific siRNA or Jak1-specific siRNA at 24 h before infection, then infected with P. yoelii YM (0.5×10⁶ iRBCs), followed by intravenous administration of recombinant mouse IFN-α and IFN-β at 18 h post infection. Knockdown efficiency of Stat1 and Jak1 siRNA are shown in FIG. 16E. Daily YM parasitemias and mortality rates are shown in FIG. 16F. Data are representative of three independent experiments and are plotted as the mean±s.d. *P<0.05, **P<0.01, ***P<0.001 vs. corresponding control. NS, not significant. † denotes mouse death.

FIGS. 17A to 17G show stage-specific role of pDCs, macrophages, and cDCs in generating IFN-α/β-induced immunity. FIG. 17A is a schematic figure to show cell (pDCs, cDCs, and macrophages) depletion at different stages of IFN-α/β cytokine production after P. yoelii YM infection. FIGS. 17B and 17C show depletion of pDCs in Mavs^{-/-} mice by anti-mPDCA-1 antibody administrated at 24 h before or 24 h after YM infection, rat IgG2b treatment served as a control. Serum levels of IFN-α and IFN-β collected at 24 h after infection are shown in FIG. 17B. Daily YM parasitemias and mortality rates of Mavs^{-/-} mice untreated or treated with anti-mPDCA-1 at the indicated time points are shown in FIG. 17C. FIGS. 17D and 17E show depletion of macrophages in Sting^{-/-} mice by clodronate (700 μg/injection) administered 2 days before or 1 day after YM infection, control liposome served as a control. Serum levels of IFN-α and IFN-β collected at 24 h after P. yoelii YM infection in Sting^{-/-} mice untreated (control liposome) or treated with clodronate at the indicated times are shown in FIG. 17D. Parasitemias and mortality rates of Sting^{-/-} mice untreated or treated with clodronate at the indicated times are shown in FIG. 17E. FIGS. 17F and 17G show WT chimeric mice were irradiated and transplanted with bone marrow cells of Mavs^{-/-}:zDC-DTR mice, then untreated or treated with DT (2.5 ng per gram body weight) as indicated at 4 days before or 1 day after P. yoelii YM infection. Serum levels of IFN-α and IFN-β collected at 24 h after infection are shown in FIG. 17F, and daily YM parasitemias and mortality rates are shown in FIG. 17G. Data are representative of three independent experiments and are plotted as the mean±s.d. *P<0.05, **P<0.01, ***P<0.001 vs. corresponding control. NS, not significant. † denotes mouse death.

FIGS. 18A to 18D show stage-specific role of T cells and B cells in generating IFN-α/β-induced immunity. FIGS. 18A and 18B show WT and Rag2^{-/-} mice infected with P. yoelii YM, followed by intravenous administration of recombinant mouse IFN-α and IFN-β at 18 h post infection. Parasitemias at day 5 and day 7 are shown in FIG. 18A. Daily YM parasitemias and mortality rates are shown in FIG. 18B. FIG. 18C shows parasitemias and host mortality rates after T cell depletion. WT and Mavs^{-/-} mice were treated with anti-CD4/CD8 antibody (300 μg/injection) every 3 days from 1 day before infection, followed by infection with P. yoelii YM. Daily YM parasitemias and mortality rates are shown. FIG. 18D shows parasitemias and host mortality rates after B cell depletion. WT and Mavs^{-/-} mice were treated with anti-CD20 antibody (250 μg/injection) every 4 days from 7 day before infection, followed by infection with P. yoelii YM. Daily YM parasitemias and mortality rates are shown. Data are plotted as the mean±s.d. and are representative of three independent experiments. **P<0.01, ***P<0.001 vs. corresponding control. NS, not significant. † denotes mouse death.

FIGS. 19A to 19E shows YM-infected mice generate adaptive immune responses after P. yoelii YM immunization. FIG. 19A is a schematic representation of procedures for mice infection. FIGS. 19B and 19C show daily parasitemias (FIG. 19B) and mortality rates (FIG. 19C) of WT (diamonds) and Mavs^{-/-} mice (squares, triangles) after primary P. yoelii YM infection (1×10⁶ iRBCs), then re-challenged with P. yoelii YM and P. yoelii N67C at dosage of 1×10⁶ iRBCs. FIG. 19D shows intracellular staining of IFN-γ were measured by FACS in splenocytes of Mavs^{-/-} mice with and without plasmodium infection as indicated, followed by stimulation with crude antigens (iRBC) infected with different malaria strain, uninfected RBCs serve as control. Statistical analysis of percentages of IFN-γ⁺/CD4⁺ specific T cells is shown. FIG. 19E shows splenocytes from plasmodium-infected or naïve Mavs^{-/-} mice cultured with crude antigens (iRBC) overnight, cell supernatants were collected for ELISA analysis. Data are representative of three independent experiments and are plotted as the mean±s.d. *P<0.05, **P<0.01, ***P<0.001 vs. corresponding control. NS, not significant. f, A working model using Mavs^{-/-} mice infected with P. yoelii YM to explain the stage-specific roles of pDCs, macrophages, cDCs and T/B cells in generating IFN-α/β-induced immunity, which can effectively protect the host from the second challenge of the same or different lethal malaria strains.

FIGS. 20A to 20E show blockage of type I IFN receptor determines the fate of P. yoelii YM infected mice. FIG. 20A shows serum levels of IFN-α, IFN-β, IL-6, and IFN-γ collected at indicated times after P. yoelii YM infection in WT mice. FIG. 20B shows serum levels of IFN-α, IFN-β, IL-6, and IFN-γ collected at 24 h after P. yoelii YM infection in Mavs^{-/-} mice untreated or treated with anti-IFNAR1 blocking antibody (500 μg) single injection at 12 h before P. yoelii YM infection. FIG. 20C shows parasitemias and mortality rates of Mavs^{-/-} mice treated with anti-IFNAR blocking antibody (500 μg, single injection at 12 h before infection), followed by infection with P. yoelii YM (2×10⁶ iRBCs). FIG. 20D shows serum levels of IFN-α, IFN-β, IL-6, and IFN-γ in WT and Ifnar^{-/-} mice at 24 h after P. yoelii YM infection. FIG. 20E shows parasitemias and mortality rates of WT and Ifnar^{-/-} mice infected with P. yoelii YM, followed by intravenous administration of recombinant mouse IFN-α and IFN-β at 18 h post infection. Data are plotted as the mean±s.d. and representative of three independent experiments. **P<0.01, ***P<0.001 vs. corresponding control. NS, not significant. † denotes mouse death.

FIGS. 21A and 21B show serum levels of IFN-α and IFN-β, parasitemias and mortality rates from BDCA2-DTR mice. WT BDCA2-DTR and Mavs^{-/-}:BDCA2-DTR mice were treated with DT (5 ng per gram body weight) as indicated at 24 h before or 24 h after P. yoelii YM infection. Serum levels of IFN-α and IFN-β collected at 24 h or 48 h after infection were measured using ELISA (FIG. 21A), Daily YM parasitemias and mortality rates were also monitored (FIG. 21B). Data are plotted as the mean±s.d. and are representative of three independent experiments. **P<0.01, ***P<0.001 vs. corresponding control. ND, not detected.

FIG. 22 shows depletion efficiency of macrophage after clodronate injection. Sting^{-/-} mice were injected with clodronate (750 μg, intraperitoneally) and peripheral blood was collected at the indicated times and stained with CD11b, F4/80 for the FACS analysis of macrophage percentage. Percentage of macrophage was compared to which in untreated mice.

FIG. 23 shows detection of malaria 18S rRNA in pDC, cDC and macrophage. The cell populations of pDC, cDC and macrophage were isolated from Mavs^{-/-} mice splenocytes at indicated times post YM infection by cell isolation kits, and then analyzed for cell-specific expression of P. yoelii 18S rRNA by PCR. Data are representative of three independent experiments.

FIGS. 24A to 24C show Mavs^{-/-} mice are resistant to P. yoelii YM single infection, but sensitive to P. yoelii N67C single infection. FIGS. 24A and 24B show daily parasitemias and mortality rates of WT (diamonds) and Mavs^{-/-} mice (triangles) after malaria one time infection (1×10⁶ iRBCs): P. yoelii YM (FIG. 24A) and P. yoelii N67C (FIG. 24B). FIG. 24C shows malaria specific IgG in serum from re-challenged Mavs^{-/-} mice, evaluated by ELISA. † denotes mouse death. Data are plotted as the mean±s.d. and are representative of three independent experiments.

FIGS. 25A and 25B show mice deficient in MAVS are sensitive to lethal P. yoelii N67C or P. berghei ANKA infection. P. b ANKA-iRBCs (FIG. 25A) or P. y N67C-iRBCs (FIG. 25B) were intraperitoneally injected into WT and Mavs^{-/-} mice. Daily parasitemias and mortality rates of WT and KO mice were measured. Similar data were obtained with repeated experiments.

FIGS. 26A and 26B show serum cytokine production in WT mice after P.y. YM, P.b. ANKA and P.y. N67C infection. FIG. 26A shows WT mice (n=5/group) infected with P. yoelii YM or P. berghei ANKA. Serum were collected at indicated times and subjected to ELISA analysis of cytokines production. FIG. 26B shows WT mice and Mavs^{-/-} mice (n=5/group) infected with P. yoelii YM or N67C. Serum were collected at indicated times and subjected to ELISA analysis of cytokines production. Data are representative of three independent experiments and are plotted as the mean±s.d.

FIGS. 27A and 27B shows blockage of type I IFN signaling or IL-6 signaling protect Mavs^{-/-} mice from lethal P. yoelii N67C or P. berghei ANKA infection. Mavs^{-/-} mice were infected with P. berghei ANKA-iRBCs (FIG. 27A) or P. yoelii N67C-iRBCs (FIG. 27B), then injected with anti-IFNAR or anti-IL6R antibodies (250 μg) at day 3 days post infection, Daily parasitemias and mortality rates were measured. Similar data were obtained with repeated experiments.**P<0.01.

### DETAILED DESCRIPTION

The term “antigen” or “immunogen” refers to any substance that stimulates an immune response. The term includes killed, inactivated, attenuated, or modified live bacteria, viruses, or parasites. The term antigen also includes polynucleotides, polypeptides, recombinant proteins, synthetic peptides, protein extract, cells (including tumor cells), tissues, polysaccharides, or lipids, or fragments thereof, individually or in any combination thereof. The term antigen also includes antibodies, such as anti-idiotype antibodies or fragments thereof, and to synthetic peptide mimotopes that can mimic an antigen or antigenic determinant (epitope).

The term “cancer” and “cancerous” refer to or describe the physiological condition in mammals that is typically characterized by unregulated cell growth. Examples of cancer include, but are not limited to, breast cancer, colon cancer, lung cancer, prostate cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, head and neck cancer, and brain cancer; including, but not limited to, gliomas, glioblastomas, glioblastoma multiforme (GBM), oligodendrogliomas, primitive neuroectodermal tumors, low, mid and high grade astrocytomas, ependymomas (e.g., myxopapillary ependymoma papillary ependymoma, subependymoma, anaplastic ependymoma), oligodendrogliomas, medulloblastomas, meningiomas, pituitary carcinomas, neuroblastomas, and craniopharyngiomas.

The term “immune response” in a host refers to the development of a humoral immune response, a cellular immune response, or a humoral and a cellular immune response to an antigen. Immune responses can usually be determined using standard immunoassays and neutralization assays, which are known in the art.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “tumor,” as used herein refers to all neoplastic cell growth and proliferation, whether malignant or benign, and all pre-cancerous and cancerous cells and tissues.

A composition for enhancing vaccine efficacy is disclosed that comprises a TLR9 or TLR7 ligand and a SOCS1 pathway antagonist in a pharmaceutically acceptable carrier.

TLR9 or TLR7 Ligand

Activation of TLR receptors has been used for the treatment of various diseases e.g activation of TLR9 by pharmaceutical products has been shown to be beneficial in treatment of allergy and oncology. Studies in mice and human indicate that the natural ligands of TLR9 are unmethylated CpG sequences in DNA molecules. Three major classes of CpG ODNs have been identified: classes A, B and C, which differ in their immunostimulatory activities. CpG-A ODNs are characterized by a PO central CpG-containing palindromic motif and a PS-modified 3′ poly-G string. They induce high IFN-α production from pDCs but are weak stimulators of TLR9-dependent NF-κB signaling and pro-inflammatory cytokine (e.g. IL-6) production. CpG-B ODNs contain a full PS backbone with one or more CpG dinucleotides. They strongly activate B cells and TLR9-dependent NF-κB signaling but weakly stimulate IFN-α secretion. CpG-C ODNs combine features of both classes A and B. They contain a complete PS backbone and a CpG-containing palindromic motif. C-Class CpG ODNs induce strong IFN-α production from pDC as well as B cell stimulation.

SOCS1 Pathway Antagonist

Gene Silencing Functional Nucleic Acids

The SOCS1 pathway antagonist can be a gene silencing functional nucleic acid. Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, triplex forming molecules, RNAi, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (K_{d}) less than or equal to 10⁻⁶, 10⁻⁸, 10⁻¹⁰, or 10⁻¹². A representative sample of methods and techniques which aid in the design and use of antisense molecules can be found in U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. It is preferred that the triplex forming molecules bind the target molecule with a K_{d} less than 10-6, 10-8, 10-10, or 10-12. Representative examples of how to make and use triplex forming molecules to bind a variety of different target molecules can be found in U.S. Pat. Nos. 5,176,996, 5,645,985, 5,650,316, 5,683,874, 5,693,773, 5,834,185, 5,869,246, 5,874,566, and 5,962,426.

External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)).

Similarly, eukaryotic EGS/RNAse P-directed cleavage of RNA can be utilized to cleave desired targets within eukarotic cells. (Yuan et al., Proc. Natl. Acad. Sci. USA 89:8006-8010 (1992); WO 93/22434 by Yale; WO 95/24489 by Yale; Yuan and Altman, EMBO J 14:159-168 (1995), and Carrara et al., Proc. Natl. Acad. Sci. (USA) 92:2627-2631 (1995)). Representative examples of how to make and use EGS molecules to facilitate cleavage of a variety of different target molecules be found in U.S. Pat. Nos. 5,168,053, 5,624,824, 5,683,873, 5,728,521, 5,869,248, and 5,877,162.

Gene expression can also be effectively silenced in a highly specific manner through RNA interference (RNAi). This silencing was originally observed with the addition of double stranded RNA (dsRNA) (Fire, A., et al. (1998) Nature, 391:806-11; Napoli, C., et al. (1990) Plant Cell 2:279-89; Hannon, G. J. (2002) Nature, 418:244-51). Once dsRNA enters a cell, it is cleaved by an RNase III-like enzyme, Dicer, into double stranded small interfering RNAs (siRNA) 21-23 nucleotides in length that contains 2 nucleotide overhangs on the 3′ ends (Elbashir, S. M., et al. (2001) Genes Dev., 15:188-200; Bernstein, E., et al. (2001) Nature, 409:363-6; Hammond, S. M., et al. (2000) Nature, 404:293-6). In an ATP dependent step, the siRNAs become integrated into a multi-subunit protein complex, commonly known as the RNAi induced silencing complex (RISC), which guides the siRNAs to the target RNA sequence (Nykanen, A., et al. (2001) Cell, 107:309-21). At some point the siRNA duplex unwinds, and it appears that the antisense strand remains bound to RISC and directs degradation of the complementary mRNA sequence by a combination of endo and exonucleases (Martinez, J., et al. (2002) Cell, 110:563-74). However, the effect of iRNA or siRNA or their use is not limited to any type of mechanism.

Short Interfering RNA (siRNA) is a double-stranded RNA that can induce sequence-specific post-transcriptional gene silencing, thereby decreasing or even inhibiting gene expression. In one example, an siRNA triggers the specific degradation of homologous RNA molecules, such as mRNAs, within the region of sequence identity between both the siRNA and the target RNA. For example, WO 02/44321 discloses siRNAs capable of sequence-specific degradation of target mRNAs when base-paired with 3′ overhanging ends, herein incorporated by reference for the method of making these siRNAs. Sequence specific gene silencing can be achieved in mammalian cells using synthetic, short double-stranded RNAs that mimic the siRNAs produced by the enzyme dicer (Elbashir, S. M., et al. (2001) Nature, 411:494 498) (Ui-Tei, K., et al. (2000) FEBS Lett 479:79-82). siRNA can be chemically or in vitro-synthesized or can be the result of short double-stranded hairpin-like RNAs (shRNAs) that are processed into siRNAs inside the cell. Synthetic siRNAs are generally designed using algorithms and a conventional DNA/RNA synthesizer. Suppliers include Ambion (Austin, Tex.), ChemGenes (Ashland, Mass.), Dharmacon (Lafayette, Colo.), Glen Research (Sterling, Va.), MWB Biotech (Esbersberg, Germany), Proligo (Boulder, Colo.), and Qiagen (Vento, The Netherlands). siRNA can also be synthesized in vitro using kits such as Ambion's SILENCER® siRNA Construction Kit.

The production of siRNA from a vector is more commonly done through the transcription of a short hairpin RNAs (shRNAs). Kits for the production of vectors comprising shRNA are available, such as, for example, Imgenex's GENESUPPRESSOR™ Construction Kits and Invitrogen's BLOCK-IT™ inducible RNAi plasmid and lentivirus vectors. Disclosed herein are any shRNA designed as described above based on the sequences for the herein disclosed inflammatory mediators.

Vaccine Compositions

The disclosed composition comprising a TLR9 or TLR7 ligand and a SOCS1 pathway antagonist can be combined with a pharmaceutically acceptable carrier or vehicle for administration as a vaccine to humans or animals. The terms “pharmaceutically acceptable carrier” or “pharmaceutically acceptable vehicle” are used herein to mean any liquid including, but not limited to, water or saline, a gel, salve, solvent, diluent, fluid ointment base, liposome, micelle, giant micelle, and the like, which is suitable for use in contact with living animal or human tissue without causing adverse physiological responses, and which does not interact with the other components of the composition in a deleterious manner.

The vaccine formulations may conveniently be presented in unit dosage form and may be prepared by conventional pharmaceutical techniques. Such techniques include the step of bringing into association the active ingredient and the pharmaceutical carrier(s) or excipient(s). In general, the formulations are prepared by uniformly and intimately bringing into association the active ingredient with liquid carriers. Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets commonly used by one of ordinary skill in the art.

Preferred unit dosage formulations are those containing a dose or unit, or an appropriate fraction thereof, of the administered ingredient. It should be understood that in addition to the ingredients particularly mentioned above, the formulations of the present invention may include other agents commonly used by one of ordinary skill in the art.

The vaccine may be administered through different routes, such as oral, including buccal and sublingual, rectal, parenteral, aerosol, nasal, intramuscular, subcutaneous, intradermal, and topical. The vaccine may be administered in different forms, including but not limited to solutions, emulsions and suspensions, microspheres, particles, microparticles, nanoparticles, and liposomes. It is expected that from about 1 to 5 dosages may be required per immunization regimen. Initial injections may range from about 1 μg to 1 mg, with a preferred range of about 10 μg to 800 μg, and a more preferred range of from approximately 25 μg to 500 μg. Booster injections may range from 1 μg to 1 mg, with a preferred range of approximately 10 μg to 750 μg, and a more preferred range of about 50 μg to 500 μg.

The volume of administration will vary depending on the route of administration. Intramuscular injections may range from about 0.1 ml to 1.0 ml.

The vaccine may be stored at temperatures of from about 4° C. to −100° C. The vaccine may also be stored in a lyophilized state at different temperatures including room temperature. The vaccine may be sterilized through conventional means known to one of ordinary skill in the art. Such means include, but are not limited to filtration, radiation and heat. The vaccine may also be combined with bacteriostatic agents, such as thimerosal, to inhibit bacterial growth.

The disclosed vaccine composition may be administered to humans, especially individuals traveling to regions where malaria is present, and also to inhabitants of those regions. The optimal time for administration of the vaccine is about one to three months before the initial infection. However, the vaccine may also be administered after initial infection to ameliorate disease progression, or after initial infection to treat the disease.

A variety of adjuvants known to one of ordinary skill in the art may be administered in conjunction with the protein in the vaccine composition. Such adjuvants include, but are not limited to the following: polymers, co-polymers such as polyoxyethylene-polyoxypropylene copolymers, including block co-polymers; polymer P1005; Freund's complete adjuvant (for animals); Freund's incomplete adjuvant; sorbitan monooleate; squalene; CRL-8300 adjuvant; alum; QS 21, muramyl dipeptide; CpG oligonucleotide motifs and combinations of CpG oligonucleotide motifs; trehalose; bacterial extracts, including mycobacterial extracts; detoxified endotoxins; membrane lipids; or combinations thereof.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: Cross-Regulation of Type I Interferon Signaling Pathways in pDC and its Utility for Vaccine Development Against Cancer

NF-κB and type I interferon (IFN) pathways play a key role in controlling the disease pathogenesis and severity of pathogen infection (Takeuchi, O. & Akira, S. Cell 140 (2010); Paludan, S. R. & Bowie, A. G. Immunity 38:870-880 (2013); Goubau, D., et al. Immunity 38:855-869 (2013); Gazzinelli, R. T., et al. Nat Rev Immunol 14:744-757 (2014)). In particular, interferon regulatory factor 3-mediated type I IFN signaling has recently been shown to inhibit or promote malaria infection, depending on the infection stage or different strains (Liehl, P. et al. Nat Med 20:47-53 (2014); Sharma, S. et al. Immunity 35:194-207 (2011); Miller, J. L., et al. Cell reports 7:436-447 (2014); Wu, J. et al. Proc Natl Acad Sci USA 111:E511-520 (2014)). However, understanding of the type I IFN innate immune response and mechanisms in vivo is still limited. Furthermore, despite recent progress, the role and regulatory mechanisms of MyD88-dependent type I IFN pathway in plasmacytoid cells (pDC) in malaria infection remain poorly understood. As shown below, mice deficient in several Toll-like receptors (TLRs) respond similarly to or are slightly more susceptible to lethal P. yoelii YM infection compared with wild-type (WT) mice, but mice deficient in cGAS, Sting, Mda5, Mavs, or Irf3 gene are resistant. The DNA/RNA sensors, cGAS and MDA5, are required for recognizing P. yoelii YM genomic DNA and RNA to activate STING- and MAVS-mediated type I IFN signaling. In contrast to the reduced IFNα/β production in Sting- Mavs- or Mda5-deficient conventional dendritic cells or macrophages, serum levels of IFNα/β in these knockout mice are 5-10-fold higher than in WT mice at 24 h after YM infection. Increased production of IFNα/β and host protection in Sting-, Mavs- or Mda5-deficient mice are completely abolished when Tlr7, Myd88, or Irf7 gene is ablated or pDCs are depleted, suggesting a major role for TLR7-MyD88-IRF7-mediated type I IFN signaling in pDCs. Further SOCS1 is identified as a key negative regulator induced by the cGAS-STING/MDA5-MAVS-mediated pathways to inhibit MyD88-dependent type I IFN signaling in pDCs, but not other cell types within the first 24 h post infection. Based on these studies, potent cancer vaccines were further developed by activating type I IFN signaling in pDCs through YM infection. Thus, the disclosed findings have identified a previously unknown innate immune regulatory mechanism by which STING/MAVS-mediated IRF3-dependent type I IFN pathway inhibits MyD88-dependent IFN signaling in pDCs in response to malaria infection, resulting in a new approach to development of therapeutic vaccines against infectious diseases and cancer.

Mice Deficient in cGAS, Sting, Mda5, Mavs or Irf3 are Resistant to P. yoelii YM Infection

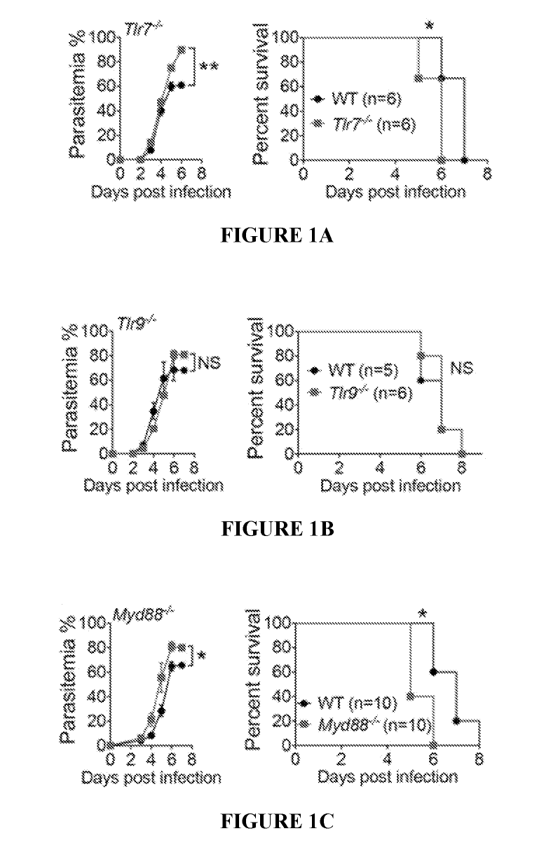

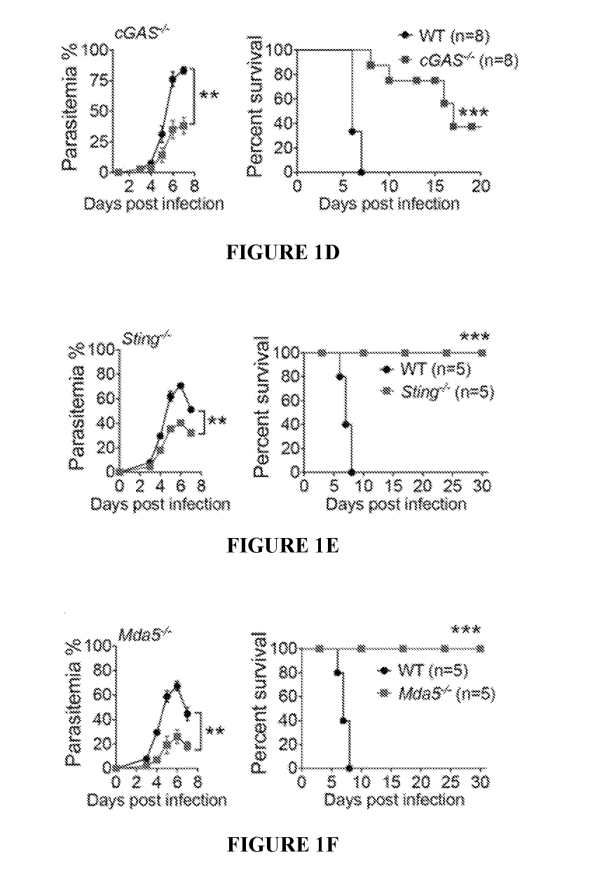

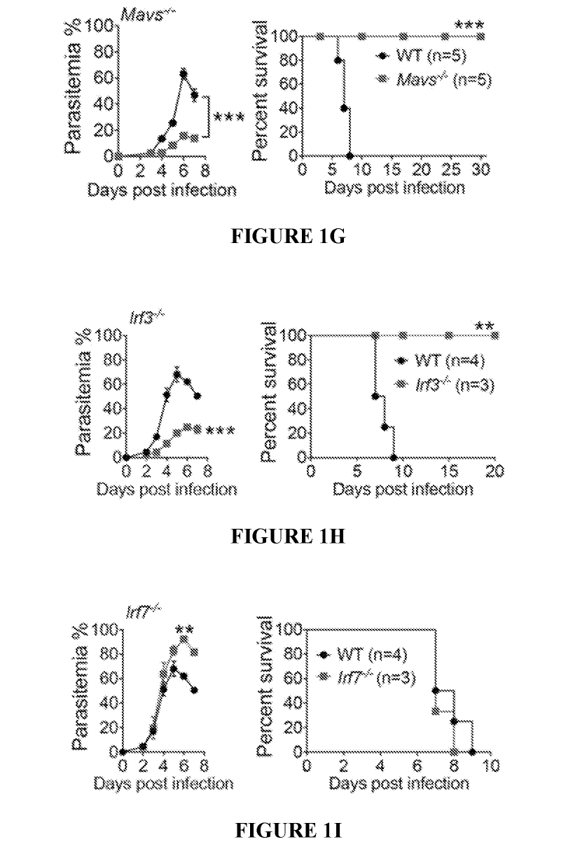

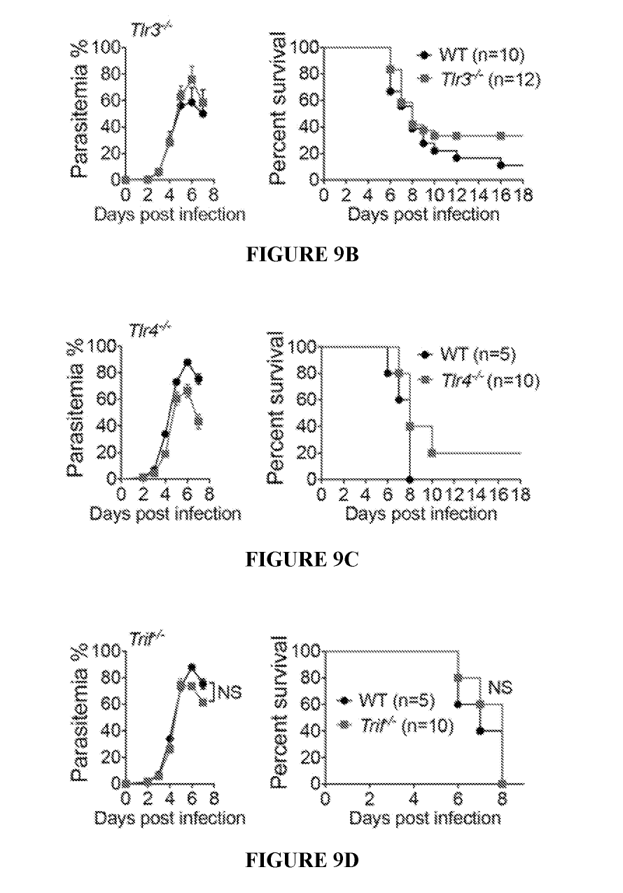

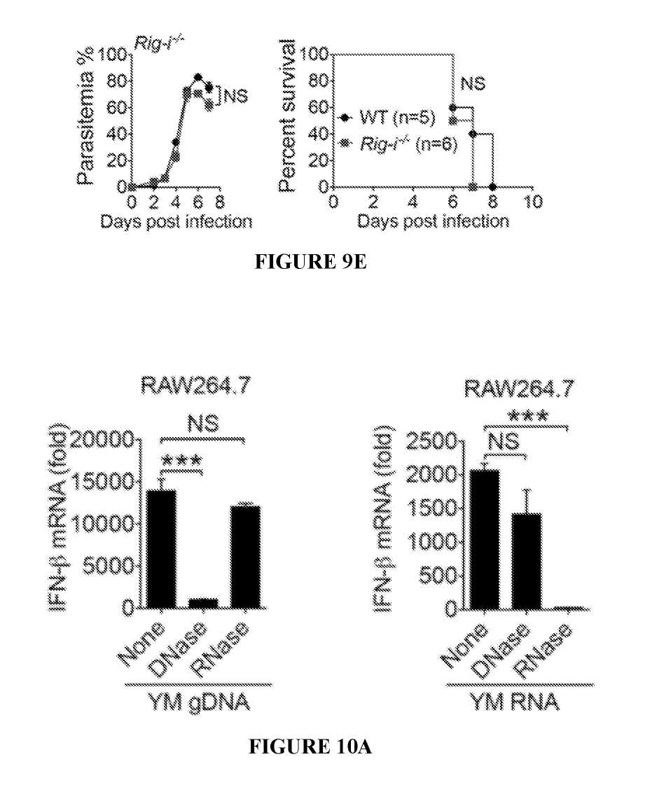

Malaria is a deadly infectious disease, affecting up to 300 million individuals worldwide, and is responsible for up to 0.5 million deaths each year (Miller, L. H., et al. Nat Med 19:156-167 (2013)). Lack of effective vaccines against malaria has been one of the major limiting factors in controlling the disease (Riley, E. M. & Stewart, V. A. Nat Med 19:168-178 (2013)). The innate immune response plays a key role in controlling the severity of malaria infection and disease pathogenesis. Recent studies show that the liver stage of infection can induce type I IFN signaling activation, either through the cytosolic pattern recognition receptor, MDA5-MAVS pathway (Liehl, P. et al. Nat Med 20:47-53 (2014)) or multiple mechanisms (Miller, J. L., et al. Cell reports 7:436-447 (2014)). Despite these important progresses, understanding of the molecular signaling pathways and mechanisms of host innate immune response to malaria during blood stage infection is still limited (Riley, E. M. & Stewart, V. A. Nat Med 19:168-178 (2013); Langhorne, J., et al. Nat Immunol 9:725-732 (2008); Stevenson, M. M. & Riley, E. M. Nat Rev Immunol 4:169-180 (2004)). To address this issue, a lethal P. yoelii YM strain was used to probe innate immune responses and potential regulatory mechanisms. Because the role of Toll-like receptors (TLRs) in malaria infection and pathogenesis remains controversial (Wu, J. et al. Proc Natl Acad Sci USA 111:E511-520 (2014); Gazzinelli, R. T. & Denkers, E. Y. Nat Rev Immunol 6:895-906 (2006); Baccarella, A., et al. Infect Immun 81:4431-4442, (2013); Gowda, N. M., et al. J Immunol 188:5073-5085 (2012); Lepenies, B. et al. Medical microbiology and immunology 197:39-44 (2008); Togbe, D. et al. Am J Pathol 170:1640-1648 (2007); Coban, C., et al. Trends Microbiol 15:271-278 (2007); Erdman, L. K., et al. Molecular and biochemical parasitology 162:105-111 (2008); Franklin, B. S. et al. Proc Natl Acad Sci USA 106:5789-5794 (2009); Wu, X., et al. J Immunol 184:4338-434 (2010); Liehl, P. & Mota, M. M. Int J Parasitol 42:557-566 (2012), experiments were first conducted to determine whether TLRs and their downstream signaling molecules play a role in controlling lethal P. yoelii YM infection. Tlr2^{-/-}, Tlr3^{-/-}, Tlr4^{-/-}, Tlr7^{-/-}, Tlr9^{-/-}, Trif^{-/-}, Myd88^{-/-} and WT mice were infected by intraperitoneal injection of P. yoelii YM-infected red blood cells (iRBCs). WT and knockout (KO) mice died within 8 days after P. yoelii YM infection (FIGS. 1a-1c, 9a-9d). Notably, Tlr7^{-/-} and Myd88^{-/-} mice showed (small but significantly) increased parasitemia and died sooner after infection than WT mice, suggesting that TLR7 and MyD88 may play a role in the control of parasitemia and host mortality.

To test whether RNA and/or DNA sensor-mediated type I IFN signaling plays a role in host immune responses to P. yoelii YM infection, cGAS^{-/-}, Sting^{-/-}, Mda5^{-/-}, and Mavs^{-/-} mice were infected by intraperitoneal injection of 1×10⁶ P. yoelii YM iRBCs. These KO mice remarkably reduced parasitemia, compared with WT mice. Sting^{-/-}, Mda5^{-/-}, and Mavs^{-/-} mice were completely resistant to YM infection, whereas cGAS^{-/-} mice showed partial protection (FIG. 1d-1g). However, mice with ablation of Rig-I gene, whose protein recognizes a short RNA with a triphosphate (PPP) moiety (Kato, H., et al. Immunol Rev 243:91-98 (2011)), showed no difference in malaria parasitemia and host death, compared with WT mice (FIG. 9e). IRF3 is a key factor for stimulating type I IFN signaling for IFN-β production in almost all cell types, whereas IRF7 is responsible for activating type I IFN signaling to produce IFN-α and IFN-β only in plasmacytoid dendritic cells (pDCs) (Liu, Y. J. Annu Rev Immunol 23:275-306 (2005); Wang, Y., et al. Immunol Rev 243:74-90 (2011)). Infection of Irf3^{-/-} and Irf7^{-/-} mice with P. yoelii YM iRBCs revealed that Irf3^{-/-} mice, not Irf7^{-/-} mice, remarkably reduced parasitemia and were resistant to P. yoelii YM infection (FIGS. 1h and 1i). Collectively, these results suggest that activation of cGAS-STING and MDA5-MAVS mediated IRF3-dependent type I IFN signaling leads to a lethal phenotype of P. yoelii YM infection.

Robust Production of IFN-α and IFN-β in cGAS^{-/-}, Sting^{-/-}, Mda5^{-/-}, Mavs^{-/-} Mice

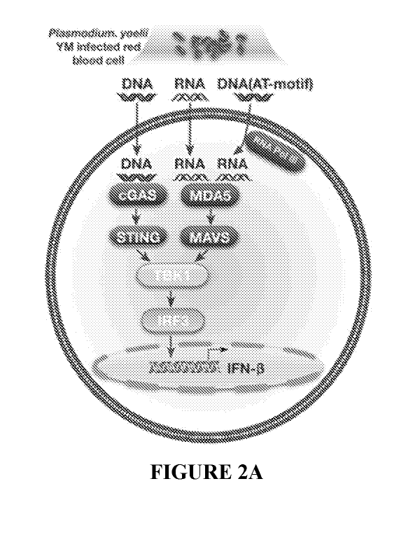

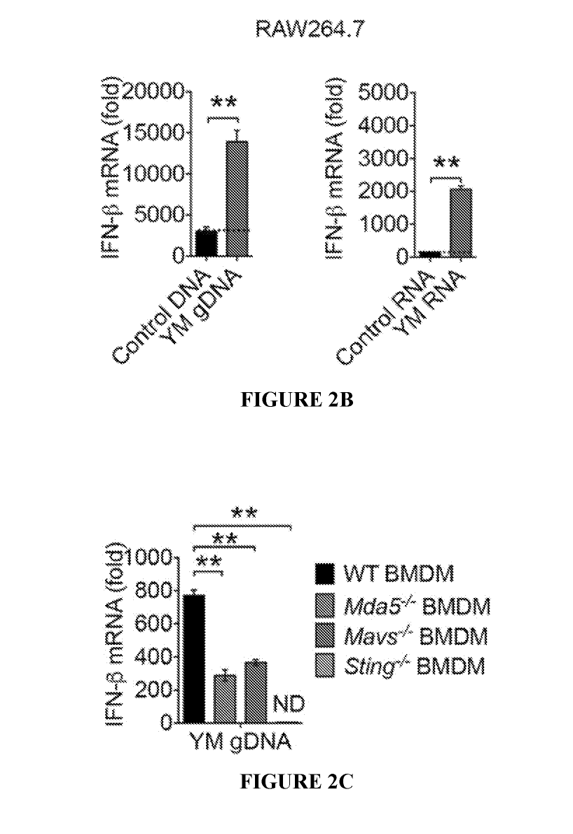

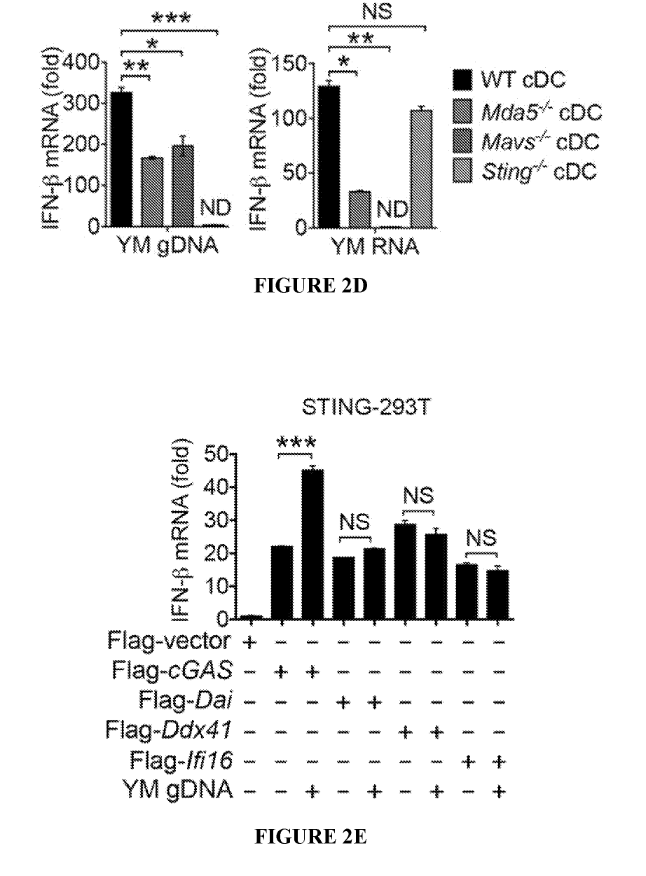

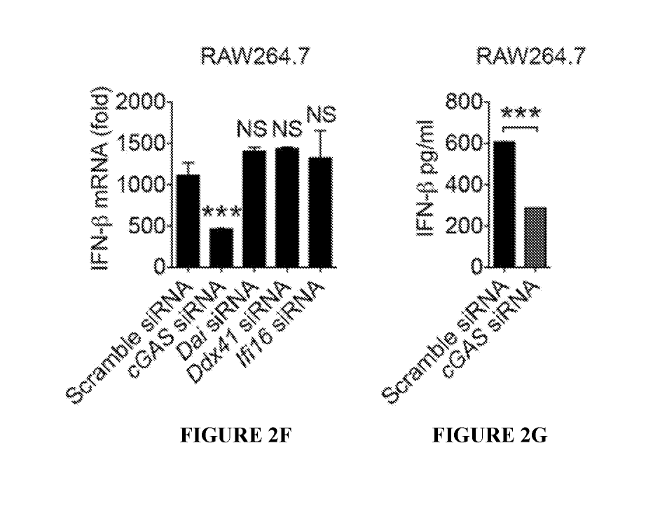

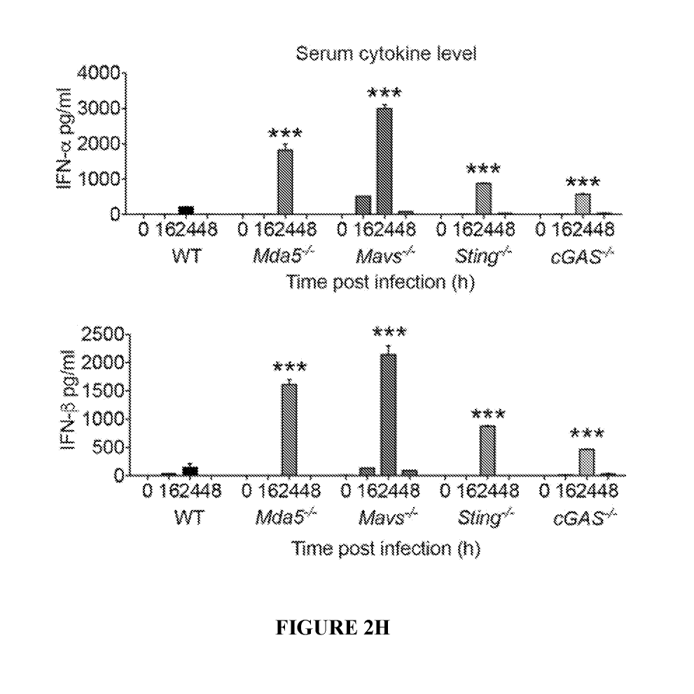

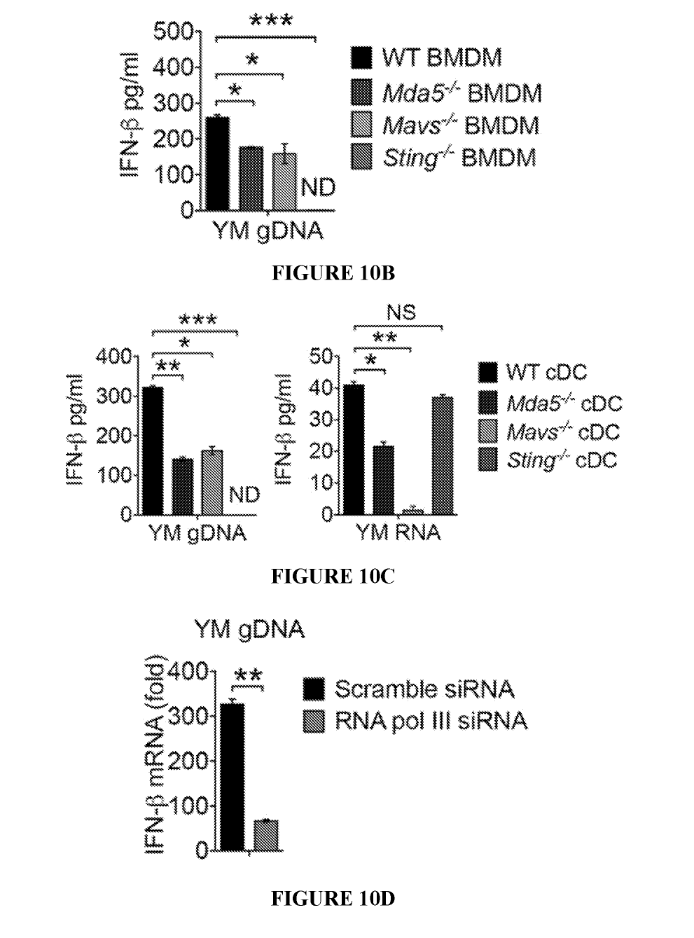

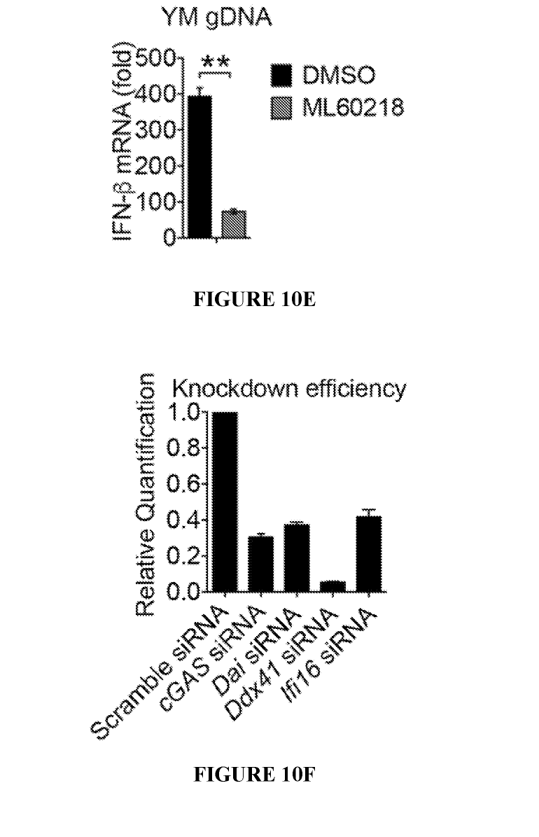

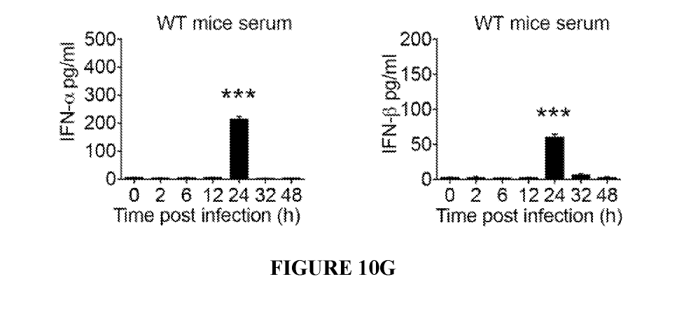

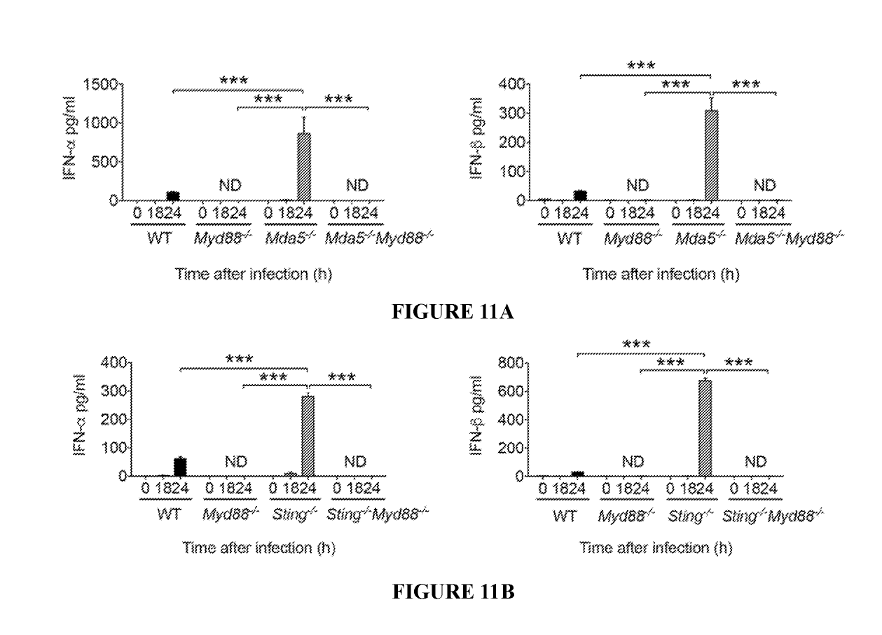

To understand the molecular mechanisms by which mice deficient in the cGAS, Sting, Mda5, or Mavs gene were resistant to P. yoelii YM infection, experiments were conducted to determine whether cGAS and MDA5 function as DNA/RNA sensors for detecting P. yoelii YM gDNAs or RNAs (FIG. 2a). Both purified P. yoelii YM gDNA and RNA could induce IFN-β mRNA expression in RAW264.7 cells (FIG. 2b). The ability of P. yoelii YM gDNA to induce type I IFN response was completely abolished upon DNase, but not RNase, treatment (FIG. 10a). Similarly, the stimulating activity of parasite RNA was abolished after treatment with RNase, but not DNase (FIG. 10a), which is in agreement with other studies showing that malaria RNA can be detected by MDA5 sensor (Liehl, P. et al. Nat Med 20:47-53 (2014); Wu, J. et al. Proc Natl Acad Sci USA 111:E511-520 (2014)). Next, bone marrow-derived macrophages (BMDM) from WT, Mda5^{-/-}, Mavs^{-/-}, and Sting^{-/-} mice were stimulated with purified P. yoelii YM gDNA, resulting in marked reduction in IFN-β mRNA and protein expression in BMDM from Mda5^{-/-} or Mavs^{-/-} mice compared with WT mice, whereas Sting^{-/-} BMDM completely lost the ability to produce IFN-β mRNA and protein (FIG. 2c, 10b). Similar results were obtained with Mda5^{-/-}, Mavs^{-/-}, and Sting^{-/-} bone marrow-derived DCs (cDCs) stimulated with P. yoelii YM gDNA, showing that either markedly reduced or completely abolished IFN-β mRNA and protein, compared with WT cells (FIG. 2d, 10c). Because RNA polymerase III can convert Poly (dA-dT) DNA into RNA for recognition by the RIG-I sensor (Zeng, W. et al. Cell 141:315-330 (2010)), it was reasoned that RNA polymerase III might convert AT-enriched malaria gDNA into long RNA for MDA5 recognition. Indeed, knockdown of RNA polymerase III with siRNA or inhibition of RNA polymerase III with inhibitor ML60218 markedly inhibited IFN-β mRNA expression (FIG. 10d-10e), thus explaining why IFN-β mRNA expression levels in Mda5^{-/-} or Mavs^{-/-} macropages and cDCs (DNA-activated STING pathway is intact) treated with P. yoelii YM gDNA were lower than those in WT cells (FIGS. 2c, 2d, 10b and 1c). Furthermore, YM RNA-stimulated IFN-β mRNA and protein expression were markedly reduced or abolished in Mda5^{-/-} or Mavs^{-/-} cDCs, compared with those in WT or Sting^{-/-} cDCs (FIG. 2d, 10c).

Although STING has been shown to play an important role in the host response to AT-rich malaria DNA (Sharma, S. et al. Immunity 35:194-207 (2011)), it is not clear which DNA sensors (cGAS, DAI, DDX41, or IFI16) are responsible for recognizing P. yoelii YM gDNA and activating STING-mediated type I IFN signaling pathway. To this end, 293T-STING stable cells were transfected with plasmids containing the gene encoding one of four putative DNA sensors (cGAS, DAI, DDX41, or IFI116), followed by P. yoelii YM gDNA stimulation. Ectopic expression of cGAS, but not DAI, DDX41, or IFI116, induced IFN-β mRNA expression, which was markedly enhanced by P. yoelii YM gDNA treatment (FIG. 2e). Consistent with this observation, knockdown (KD) of cGAS, but not DAI, DDX41, or IFI116, by specific siRNAs in RAW264.7 cells markedly reduced IFN-β mRNA and protein after P. yoelii YM gDNA treatment (FIG. 2f-2g, 10f). These data suggest that cGAS functions as a DNA sensor for detecting YM gDNA and inducing STING-mediated type I IFN signaling pathway.

Previous studies showed that type I IFN signaling inhibits type II IFN (IFN-γ) or adaptive immune responses upon malaria and viral infection (Teles, R. M. et al. Science 339:1448-1453 (2013); Palomo, J. et al. Eur J Immunol 43:2683-2695 (2013); Haque, A. et al. Eur J Immunol 41:2688-2698 (2011); Teijaro, J. R. et al. Science 340:207-211 (2013); Wilson, E. B. et al. Science 340:202-207 (2013)). To this end, the serum levels of IFN-α and IFN-β in WT mice were examined during YM infection, where cytokines were increased and peaked at 24 h post infection, and then completely disappeared in WT mice (FIG. 10g). However, the serum levels of IFN-α and IFN-β in Mda5^{-/-}, Mavs^{-/-}, Sting^{-/-}, and cGAS^{-/-} mice were much higher (5-10 fold) than in WT mice at 24 h after P. yoelii YM infection (FIG. 2h), suggesting that Mda5, Mavs, Sting or cGAS deficiency enhances type I IFN production in vivo after YM infection.

Role of TLR7-MyD88-IRF7 Type I IFN Signaling in Robust Production of IFN-α and IFN-β

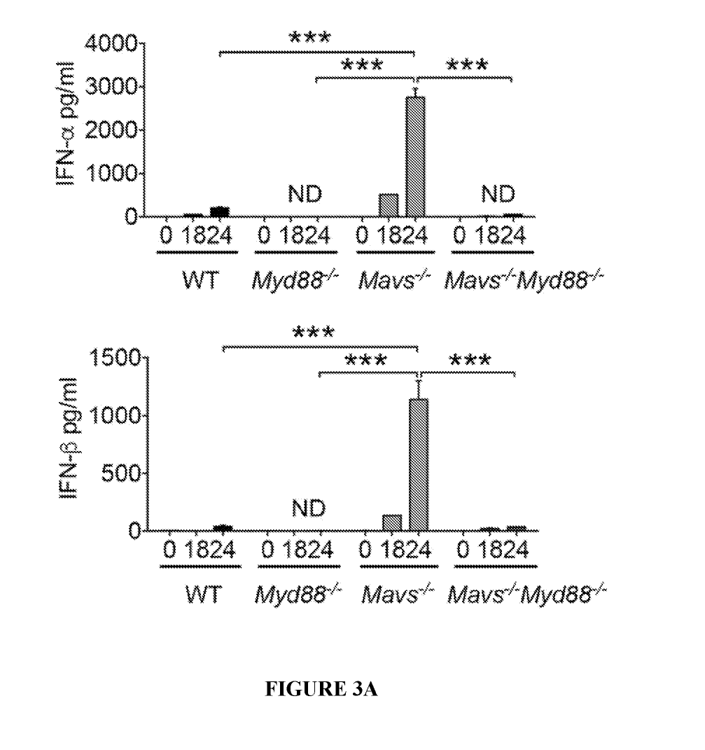

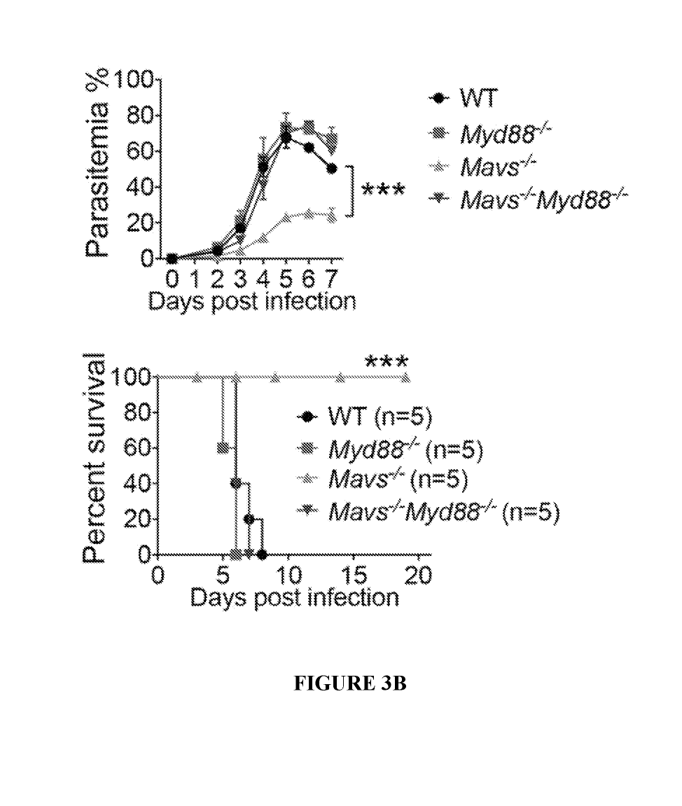

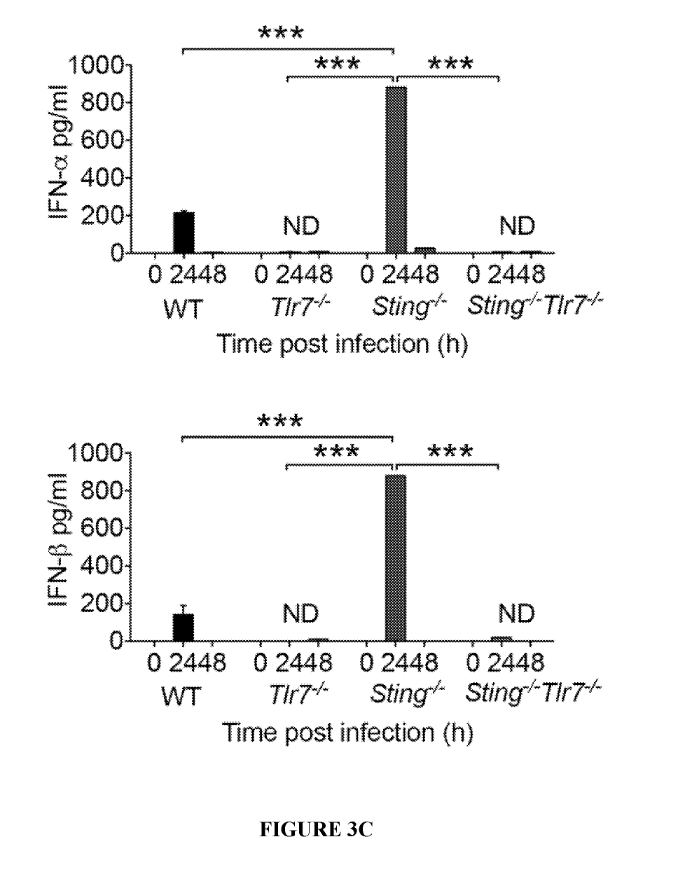

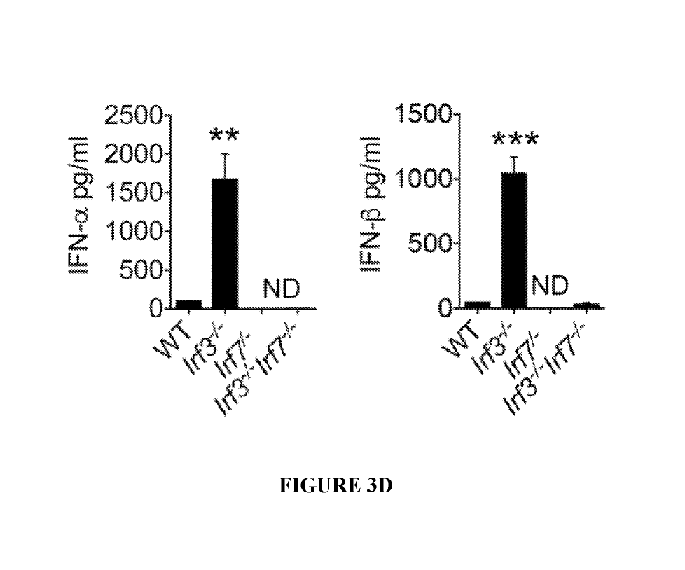

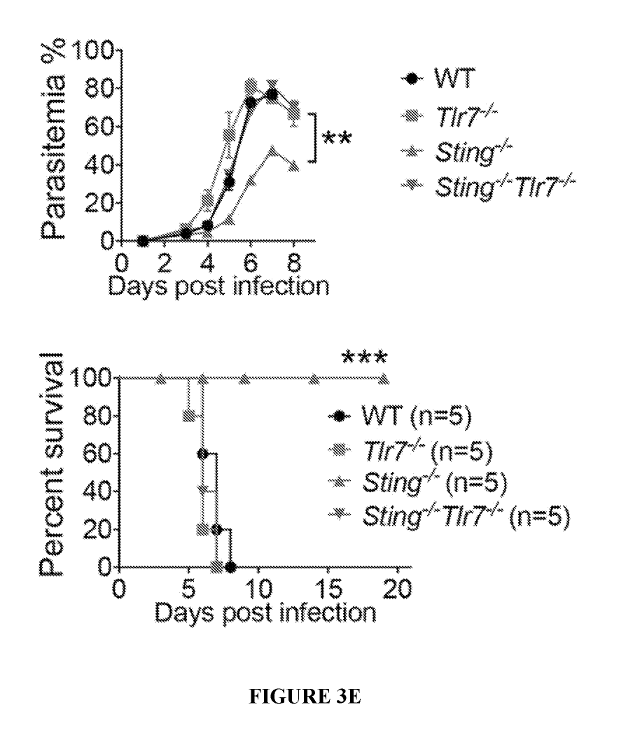

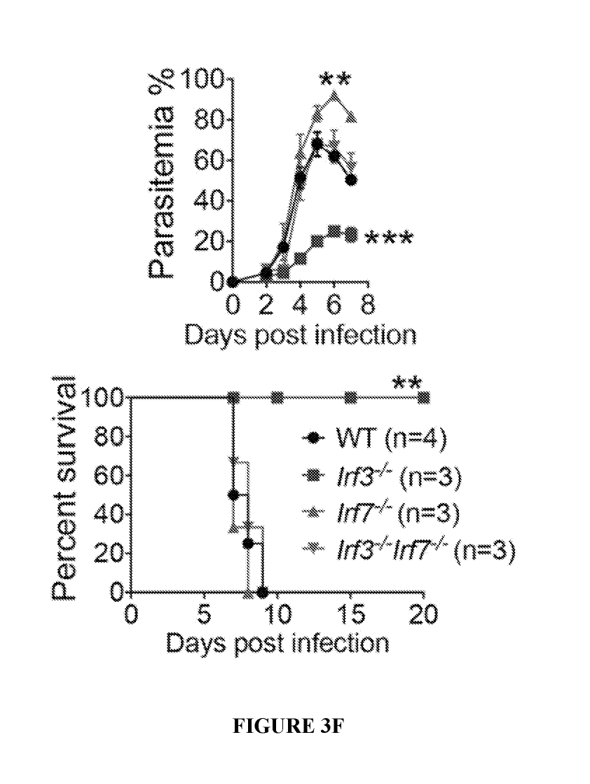

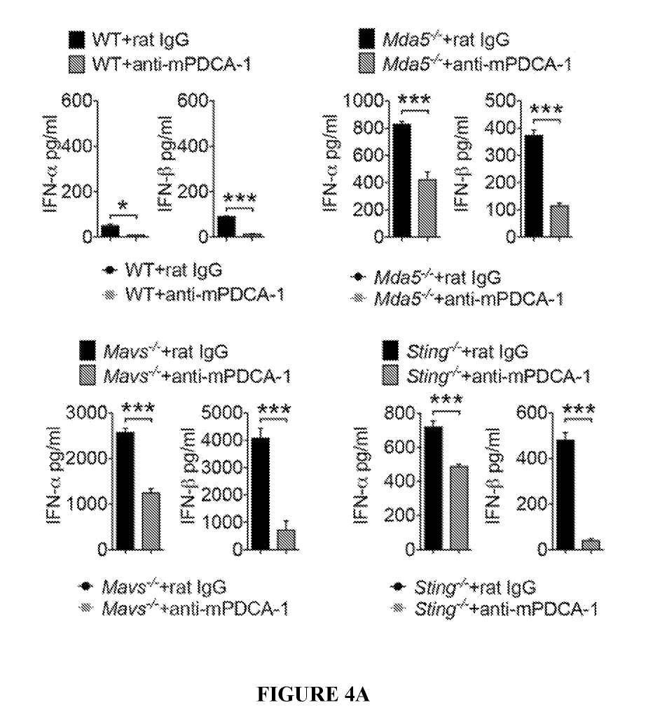

Since the in vitro data using Mda5^{-/-}, Mavs^{-/-}, and Sting^{-/-} cDCs and macrophages after malaria gDNA or RNA stimulation show markedly reduced or completed loss of IFN-α and IFN-β, and thus could not explain high serum levels of these cytokines in Mda5^{-/-}, Mavs^{-/-}, and Sting^{-/-} mice, experiments were conducted to identify the key signaling pathways that are responsible for the robust production of IFN-α and IFN-β in vivo at 24 h after YM infection. Because Tlr7^{-/-}, Myd88^{-/-}, and Irf7^{-/-} mice were more sensitive than WT mice in response to YM infection (FIG. 1), experiments were conducted to determine whether ablation of the Tlr7, Myd88, or Irf7 gene could reduce IFN-α and IFN-β production and reverse the resistant phenotypes of Mda5^{-/-}, Mavs^{-/-} and Sting^{-/-} mice to YM infection. Mda5^{-/-}:Myd88^{-/-}, Mavs^{-/-}:Myd88^{-/-}, and Sting^{-/-}:Myd88^{-/-} double knockout (DKO) mice were generated, and then WT, single KO, and DKO mice were infected with P. yoelii YM. As expected, serum levels of IFN-α and IFN-β were very high in single KO (Mda5^{-/-}, Mavs^{-/-}, and Sting^{-/-}) mice, but low in WT mice. Strikingly, no IFN-α and IFN-β was detected in the sera of DKO (Mda5^{-/-}:Myd88^{-/-}, Mavs^{-/-}:Myd88^{-/-}, and Sting^{-/-}:Myd88^{-/-}) or in the sera of Myd88 single KO mice (FIG. 3a, 11a-11b), suggesting a critical role for MyD88 in the rapid production of high amounts of these cytokines in response to P. yoelii YM infection. Consistent with the cytokine production data, MyD88 ablation sensitized single KO (Mda5^{-/-}, Mavs^{-/-}, and Sting^{-/-}) mice to YM strain-induced high parasitemia and host lethality (FIG. 3b, 11c-11d). Taken together, these results suggest that the rapid production of high levels of type I IFN in the Mda5^{-/-}, Mavs^{-/-}, and Sting^{-/-} mice after P. yoelii YM infection is MyD88-dependent.

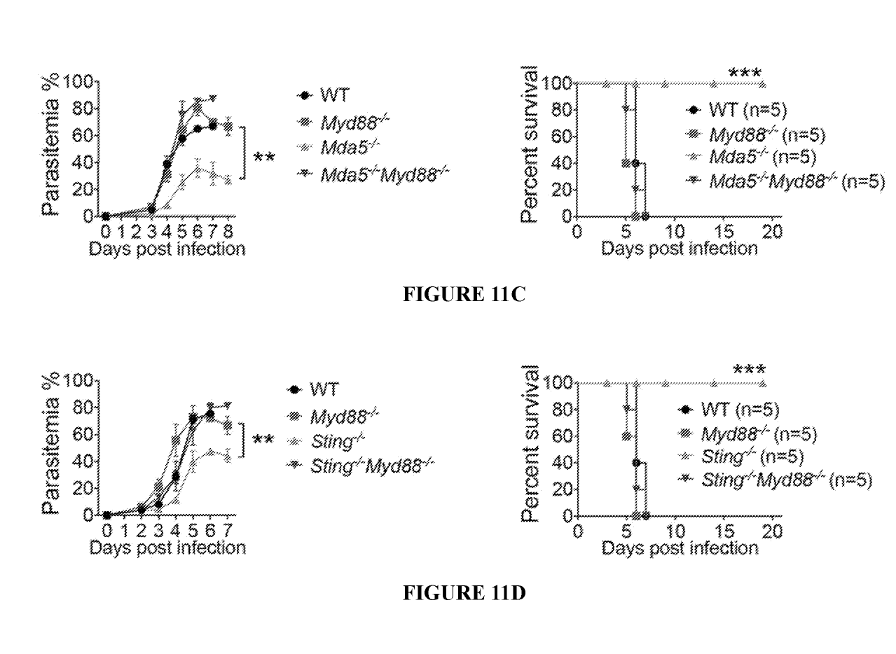

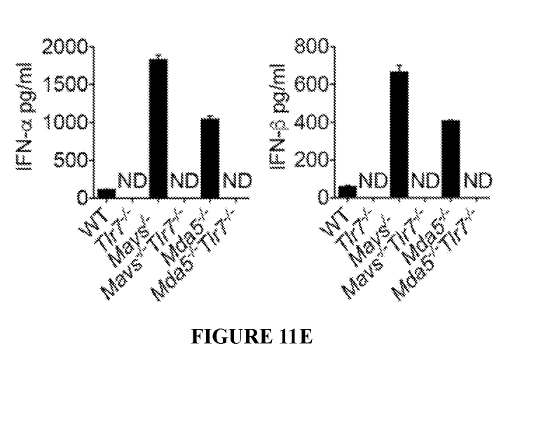

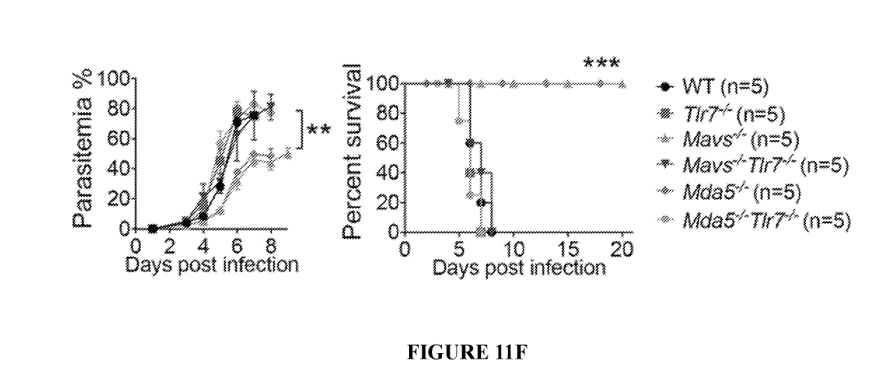

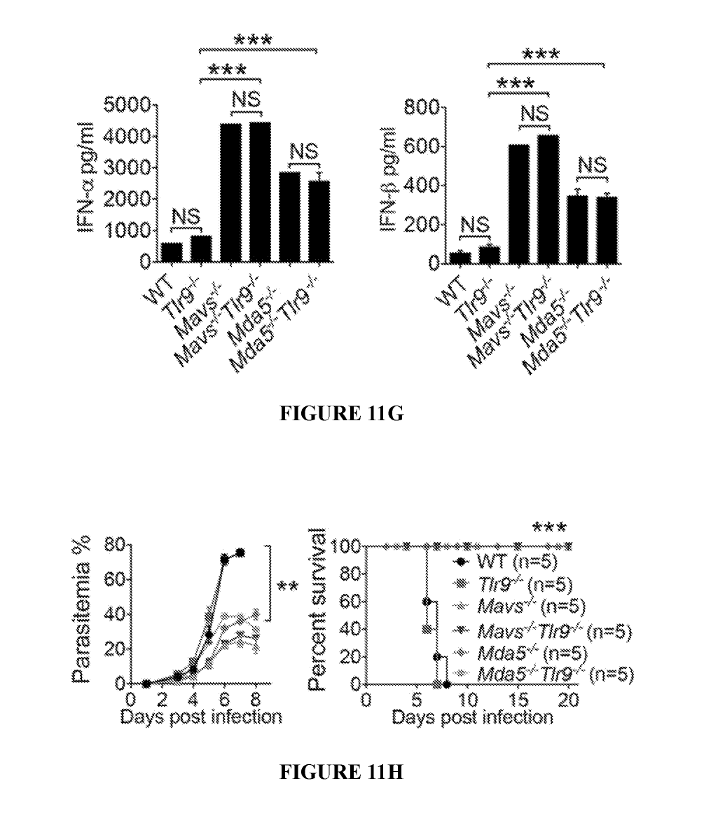

Using a similar strategy, serum levels of IFN-α and IFN-β were shown to be very high in Sting^{-/-} and Irf3^{-/-} mice, but not detectable in the sera of Tlr7^{-/-}, Irf7^{-/-}, Sting^{-/-}:Tlr7^{-/-} and Irf3^{-/-}:Irf7^{-/-} mice (FIG. 3c-3d). TLR7 or IRF7 ablation converted the resistant phenotypes (low parasitemia and host survival) of Sting^{-/-} and Irf3^{-/-} mice to the sensitive phenotypes (high parasitemia and host death) of Sting^{-/-}:Tlr7^{-/-} and Irf3^{-/-}:Irf7^{-/-} mice after P. yoelii YM infection (FIG. 3e-3f). Similar results were observed in Mavs^{-/-}:Tlr7^{-/-} and Mda5^{-/-}:Tlr7^{-/-} mice (FIG. 11e-11f). By contrast, no difference in IFN-α and IFN-β, parasitemia, or host death was observed between Mda5^{-/-} and Mavs^{-/-} mice versus Mda5^{-/-}:Tlr9^{-/-} and Mavs^{-/-}:Tlr9^{-/-} mice (FIG. 11g-11h), indicating that TLR9 is not required for cytokine production and protective immunity. Taken together, these results suggest that TLR7-MyD88-IRF7 molecules constitute key components of MyD88-dependent type I IFN signaling, which has been shown to operate in only pDC for production of large amounts of IFN-α and IFN-β, generally in response to viral infection (Liu, Y. J. Annu Rev Immunol 23:275-306 (2005); Wang, Y., et al. Immunol Rev 243:74-90 (2011)).

PDCs are the Major Source of Type I IFN Cytokine Production

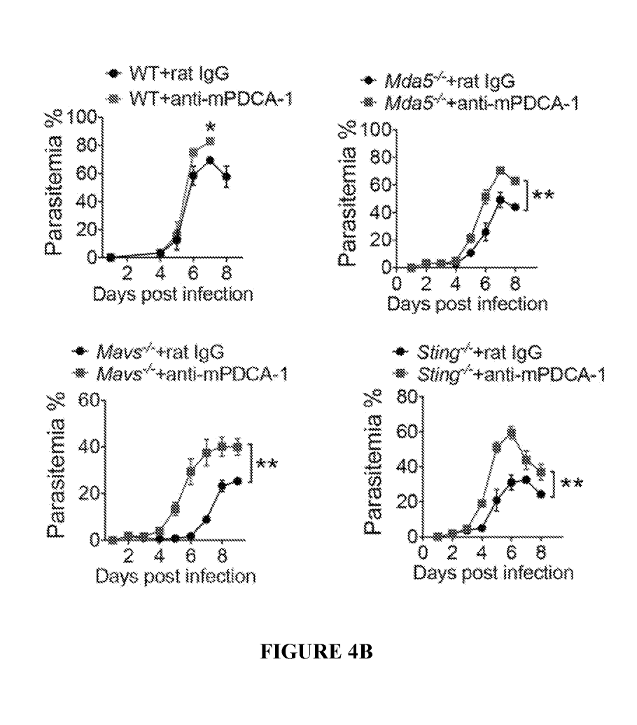

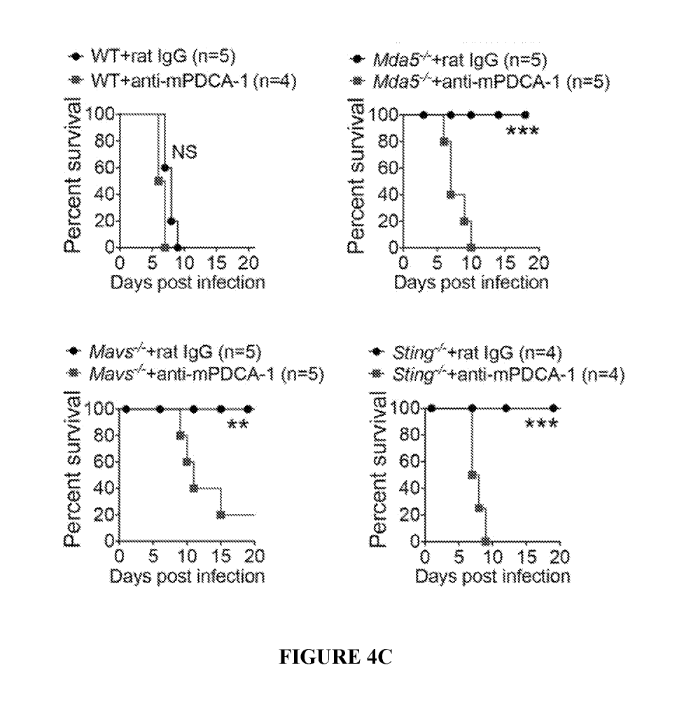

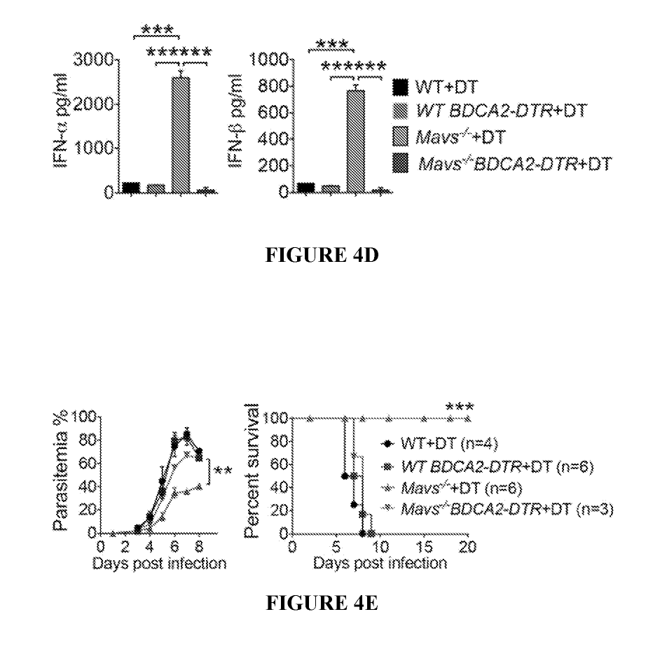

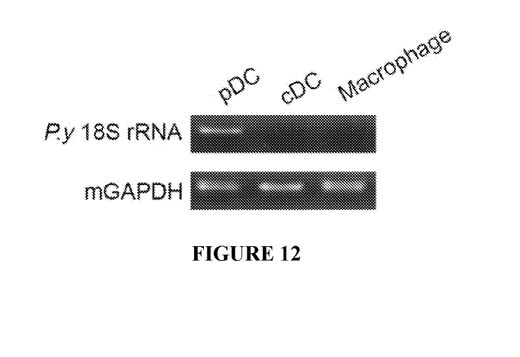

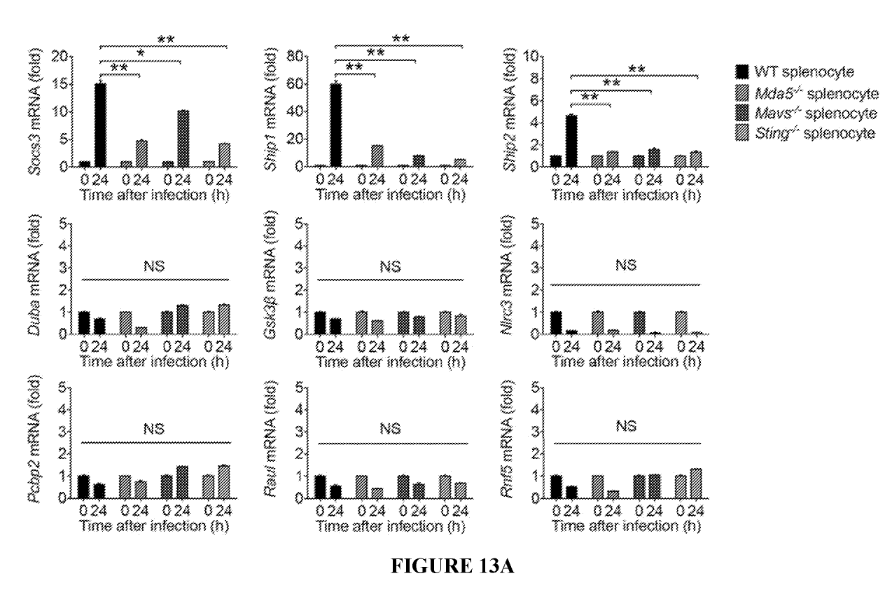

To determine a role of pDC in malaria infection, experiments were conducted to determine whether pDCs could be preferentially targeted by P. yoelii YM infection. To this end, WT mice were infected with P. yoelii YM infection and cDCs, macrophages, and pDCs isolated at 18 h post infection to detect malaria 18S rRNA by PCR with malaria-specific primers. Malaria 18S rRNA could be detected in pDCs, but not in macrophages and cDCs (FIG. 12), suggesting that pDCs, but not macrophages and cDCs, are involved in detecting P. yoelii YM infection within the first 18 h. To directly demonstrate that pDCs are the major source of type I IFN cytokine production, pDCs were depleted in WT, Mda5^{-/-}, Mavs^{-/-}, and Sting^{-/-} mice by injection of the monoclonal antibody, mPDCA-1, at 12 h prior to P. yoelii YM infection and at 12 h after infection. Depletion of pDCs significantly reduced serum levels of IFN-α and IFN-β in WT, Mda5^{-/-}, Mavs^{-/-}, or Sting^{-/-} mice, compared with control antibody treatment (FIG. 4a). These results suggest that IFN-α and IFN-β are primarily produced by pDCs. Most importantly, pDC depletion by mPDCA-1 treatment markedly increased parasitemias and mortality in Mda5^{-/-}, Mavs^{-/-}, and Sting^{-/-} mice infected with P. yoelii YM, compared with control antibody-treated mice (FIG. 4b-4c). Taken together, these results clearly suggest that pDCs are responsible for the early and rapid production of type I IFN in the Mda5^{-/-}, Mavs^{-/-}, and Sting^{-/-} mice, which, in turn, activates type I IFN downstream signaling for potent innate immunity against P. yoelii YM infection.

To further validate the role of pDCs in type I IFN production and mouse protection after P. yoelii YM infection, pDCs were genetically ablated in vivo by generating Mda5^{-/-}:BDCA2-DTR, Mavs^{-/-}:BDCA2-DTR, and Sting^{-/-}:BDCA2-DTR mice, as previously described for BDCA2-DTR mice (Swiecki, M., et al. Immunity 33:955-966 (2010)). Upon diphtheria toxin (DT) administration, pDCs were depleted in Mavs^{-/-}:BDCA2-DTR mice, but not in Mavs^{-/-} mice. These treated mice were then infected with P. yoelii YM. Treatment of Mavs^{-/-}:BDCA2-DTR mice with DT markedly reduced IFN-α and IFN-β compared with Mavs^{-/-} mice treated with DT (FIG. 4d). More importantly, DT-treated Mavs^{-/-}:BDCA2-DTR mice were sensitive to P. yoelii YM infection based on parasitemia and survival, compared with DT-treated Mavs^{-/-} mice, which remained resistant to P. yoelii YM infection (FIG. 4e). Taken together, these results suggest that pDCs are required for robust production of type I IFN cytokines and host protective immunity against P. yoelii YM infection.

SOCS1 is Induced by STING/MAVS-Mediated Type I Signaling and Inhibits MyD88-Dependent Type I IFN Signaling in pDCs

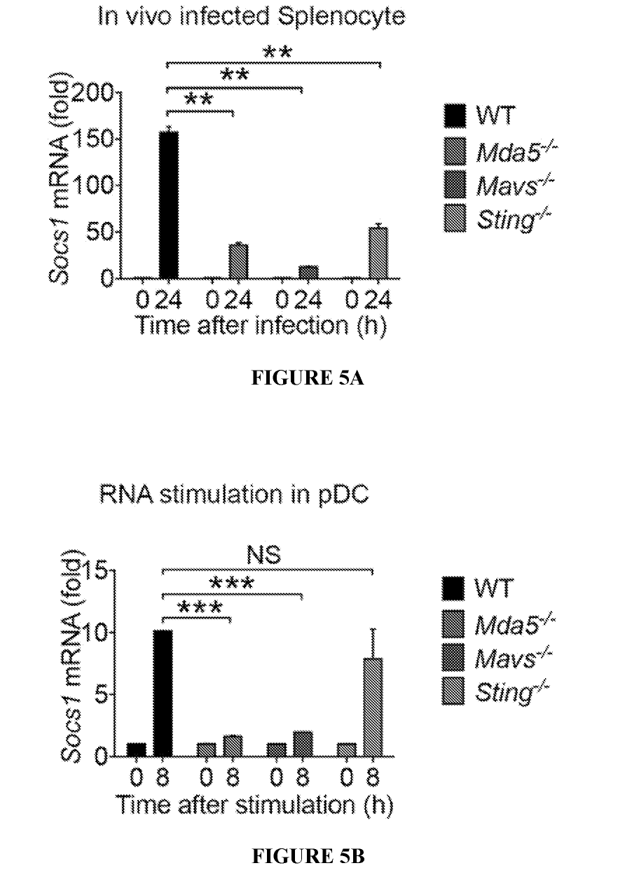

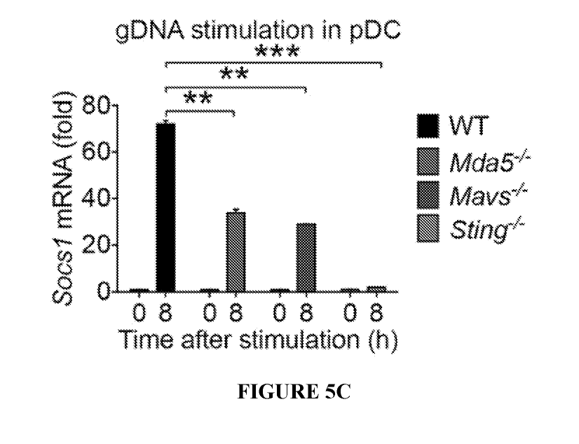

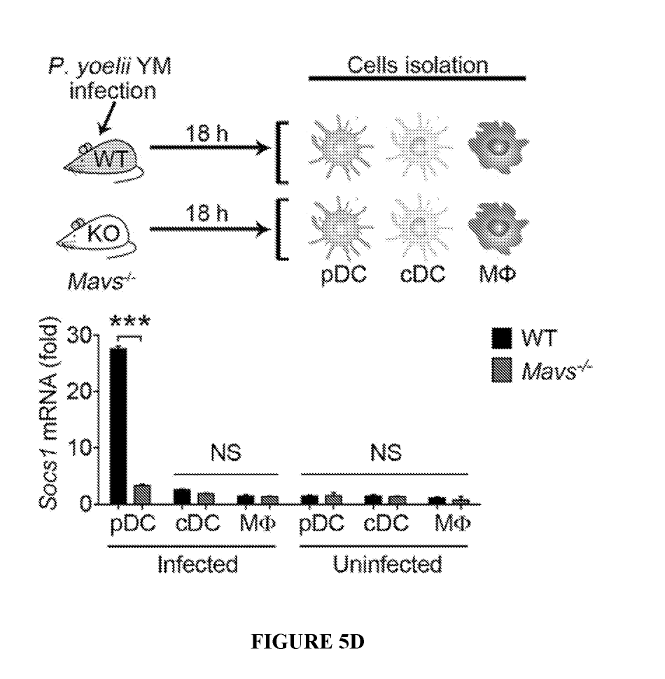

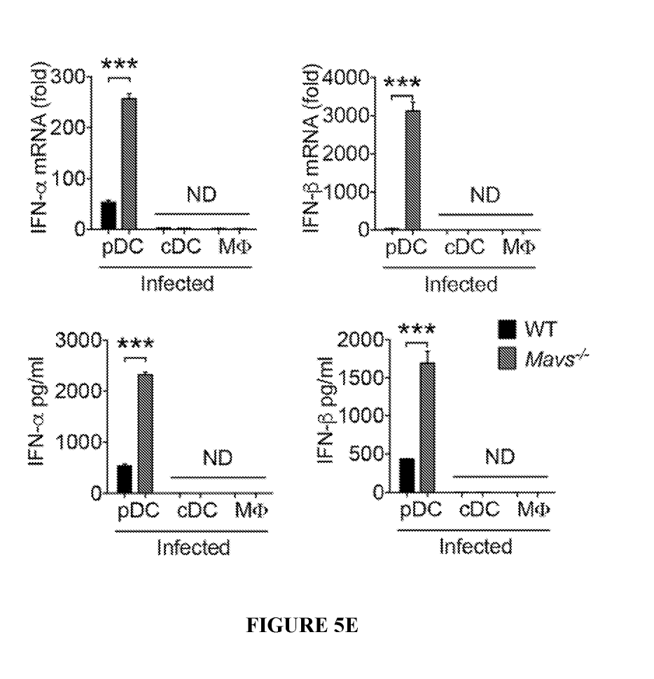

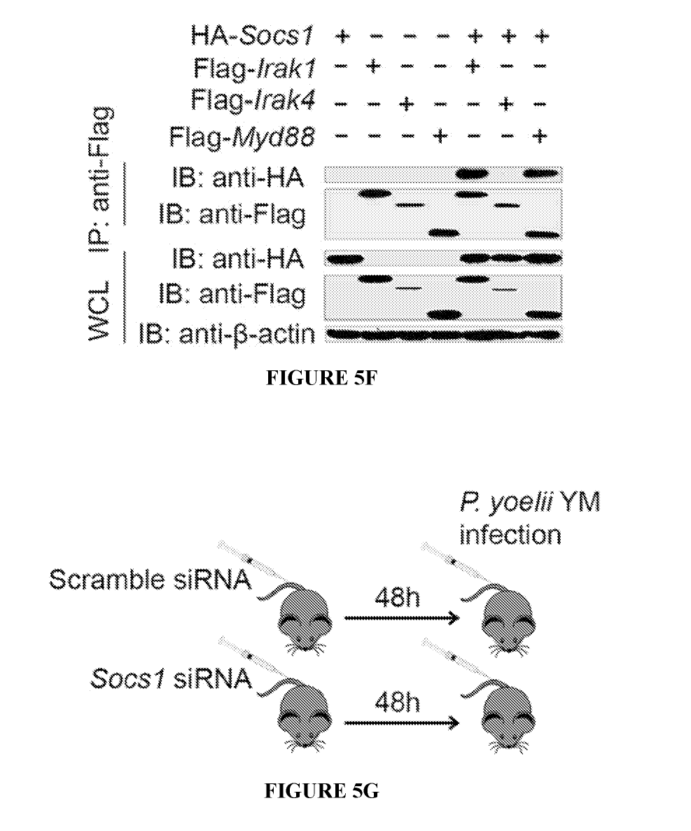

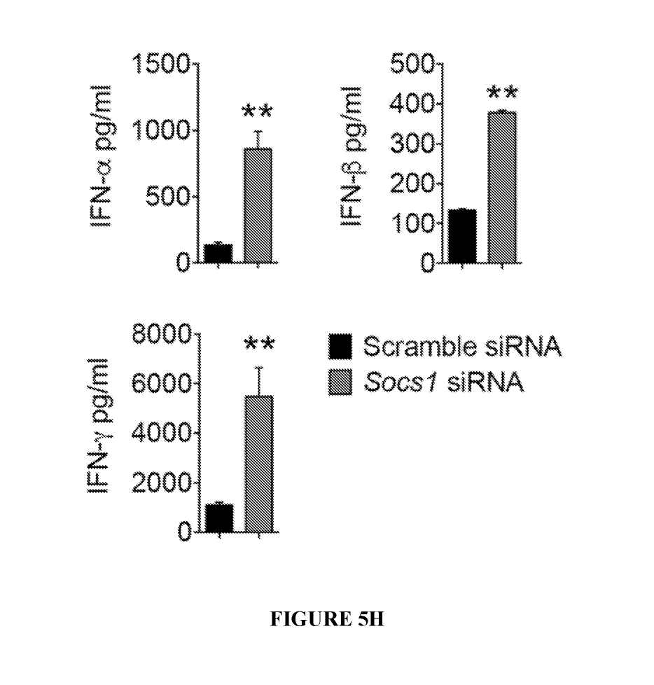

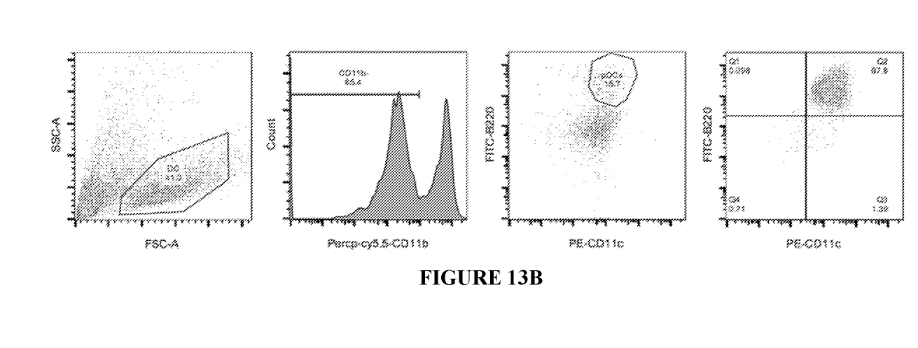

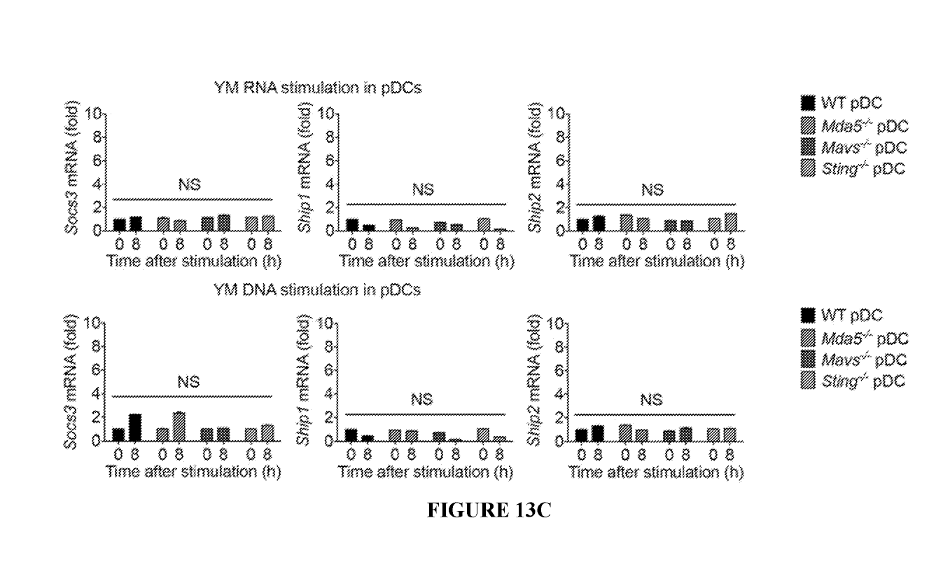

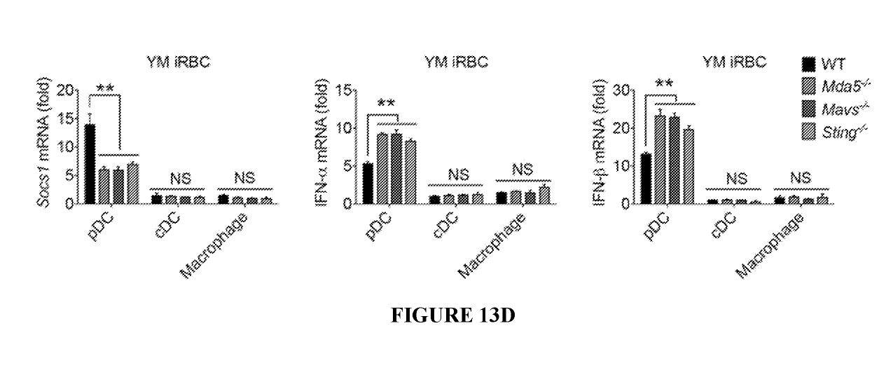

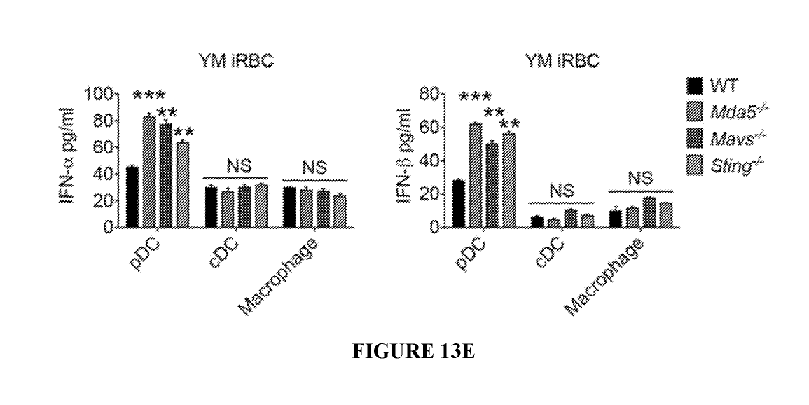

To further elucidate the mechanisms by which ablation of Mda5, Mavs, or Sting markedly enhanced MyD88-dependent type I IFN production in pDCs, it was hypothesized that activation of STING- and MAVS-mediated type I IFN signaling by P. yoelii YM may induce expression of negative regulators, such as SOCS1, SOCS3, SHIP1, and SHIP2, which, in turn, may inhibit MyD88-dependent type I IFN signaling activation in pDCs. To test this possibility, the expression pattern of several negative regulators was examined in freshly isolated splenocytes from P. yoelii YM-infected WT and KO (Mda5^{-/-}, Mavs^{-/-}, and Sting^{-/-}) mice, demonstrating that the mRNA levels of Socs1, Socs3, Ship1, and Ship2 were markedly increased in WT, but little or low in KO splenocytes (FIG. 5a, 13a). By contrast, no appreciable changes in the expression of other potential negative regulators, such as Duba, Gsk3β, Nlrc3, Pcbp2, Raul, and Rnf5, was observed between YM-infected WT and KO mice (FIG. 13a). To further assess the expression of these negative regulators in pDCs, pDCs were purified from Flt3L-induced bone morrow-derived DCs of WT and KO mice by FACS (FIG. 13b), and were stimulated with P. yoelii YM RNA or gDNA. SOCS1 was the only negative regulator showing a markedly increase in gene expression in WT pDCs, but significantly lower in KO (Mda5^{-/-}, Mavs^{-/-}, and Sting^{-/-}) pDCs (FIG. 5b-5c). No appreciable differences in expression of other negative regulators Socs3, Ship1, and Ship2 were observed between WT and KO pDCs after YM RNA or gDNA stimulation (FIG. 13c). To further substantiate these findings, pDCs, macrophages, and cDCs were freshly purified from YM-infected WT and Mavs^{-/-} mice (18 h after infection), and SOCS1 expression was directly assessed in these cell populations, demonstrating that SOCS1 mRNA expression levels in pDCs from WT mice were significantly higher than pDCs from Mavs^{-/-} mice (FIG. 5d). Notably, no difference in expression of SOCS1 was detected in macrophages and cDCs from WT and Mavs^{-/-} mice, compared with these cells from untreated mice (FIG. 5d). Importantly, SOCS1 expression levels were inversely correlated with IFN-α and IFN-β mRNA and protein expression in pDCs from YM-infected WT and Mavs^{-/-} mice (FIG. 5e). Similar results were obtained using purified pDCs, cDCs and macrophages treated in vitro with YM-iRBCs (FIGS. 13d and 13e). Collectively, these results suggest that macrophages and cDCs may not be involved in anti-malaria immunity within the first 18-24 h post infection.

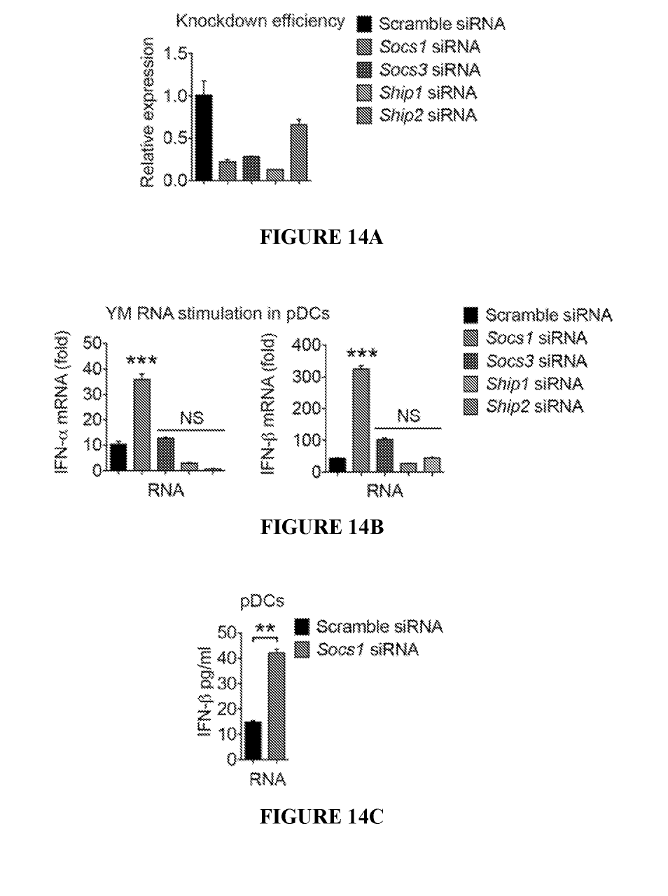

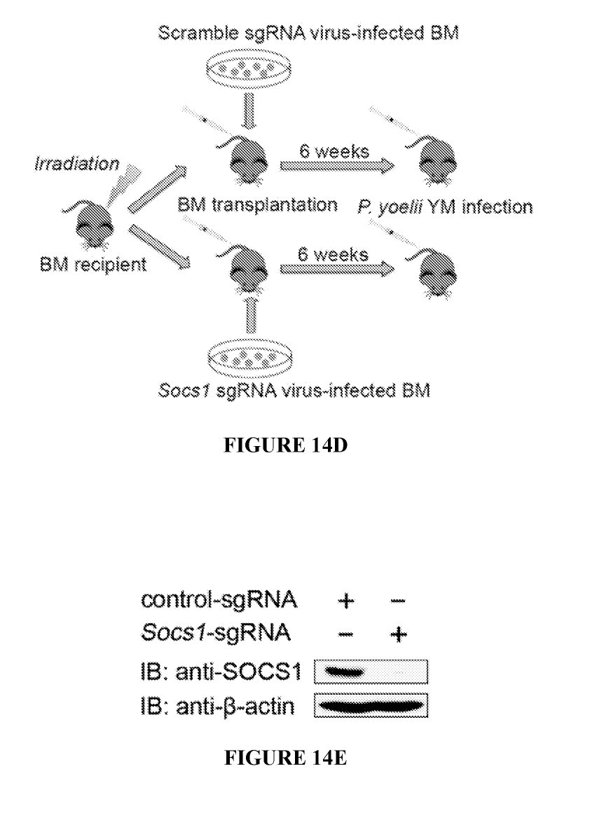

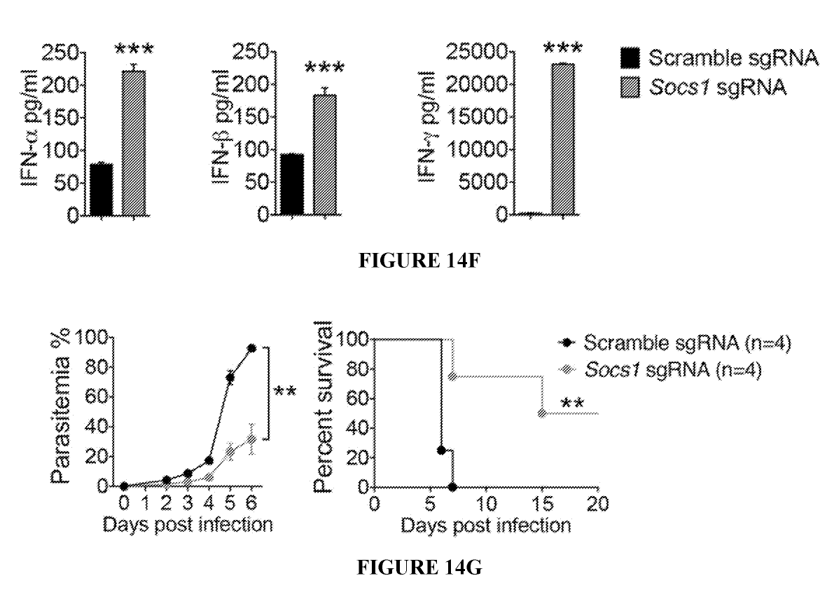

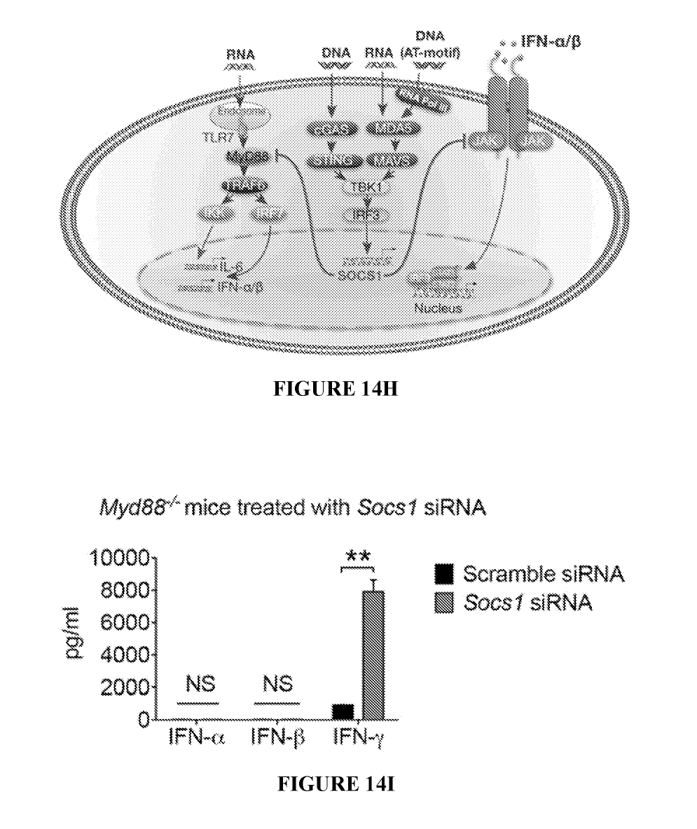

Next, experiments were conducted to determine how SOCS1 inhibits MyD88-dependent type I IFN signaling pathway. Although previous studies showed that SOCS1 inhibits the NF-κB signaling pathway by interacting with IRAK1 adaptor protein (Nakagawa, R. et al. Immunity 17:677-687 (2002)), the involvement of SOCS1 in MyD88-mediated type I IFN signaling is not known. To determine whether SOCS1 directly interacts with MyD88, co-immunoprecipitation of 293T cells expressing Socs1 plus Myd88, Irak1 or Irak4, was performed, demonstrating that SOCS1 directly interacted with MyD88 (FIG. 5f), in addition to the previously reported IRAK protein (Nakagawa, R. et al. Immunity 17:677-687 (2002)). To determine whether STING-, MDA5- and MAVS-induced SOCS1 is responsible for inhibition of MyD88-dependent type I IFN production in pDCs, Socs1, Socs3, Ship1, and Ship2 were knocked down in WT pDCs by siRNA transfection (FIG. 14a) and IFN-α and IFN-β production was determined after YM RNA stimulation. KD of Socs1, but not Socs3, Ship1 or Ship2, resulted in a significant increase in IFN-β and IFN-β mRNA as well as IFN-β protein after YM RNA stimulation (FIGS. 14b and 14c). These results suggest that STING- or MAVS-mediated type I IFN signaling induces SOCS1 expression, which, in turn, inhibits MyD88-dependent type I IFN production in pDCs.

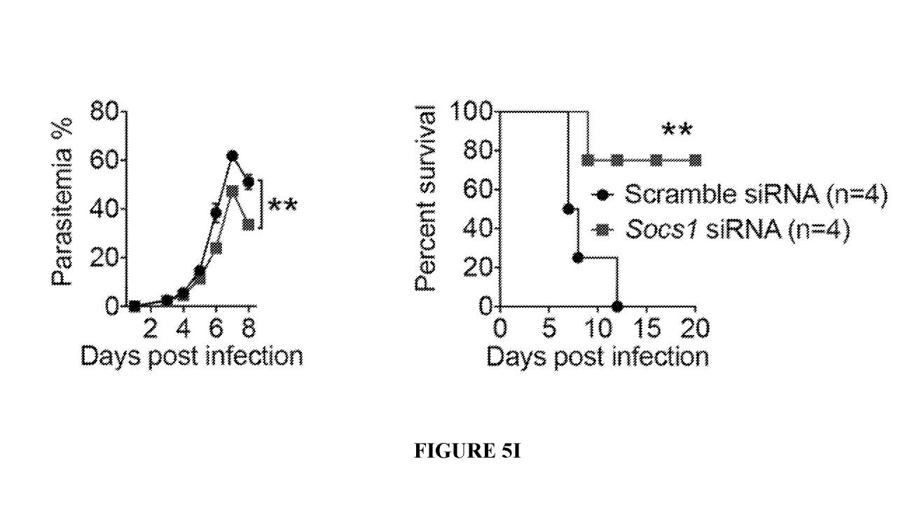

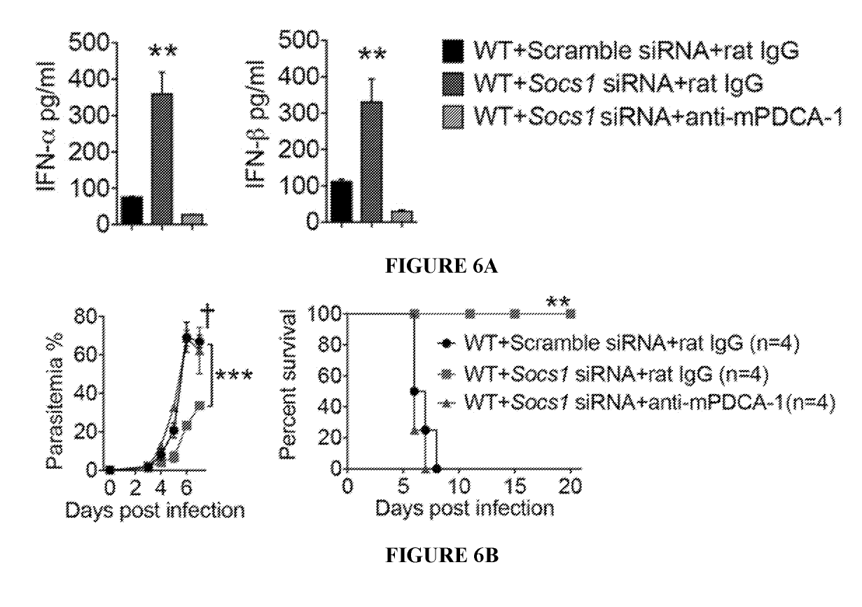

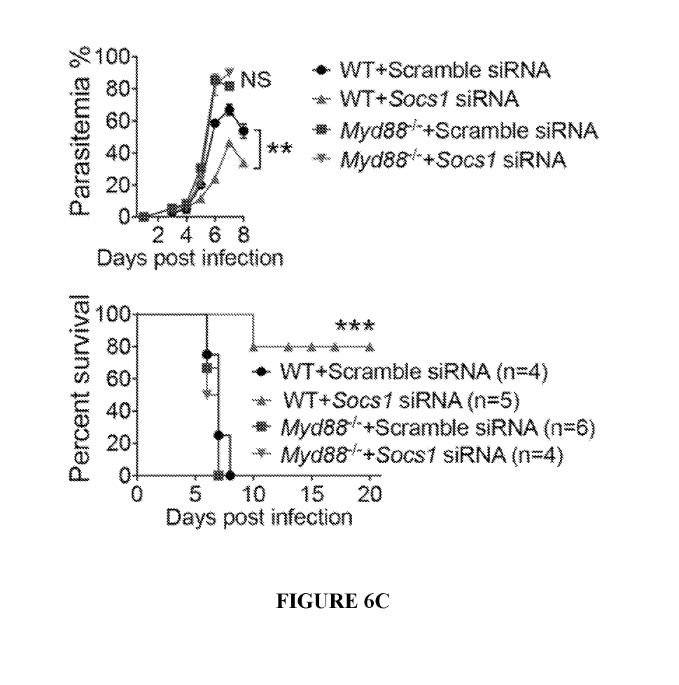

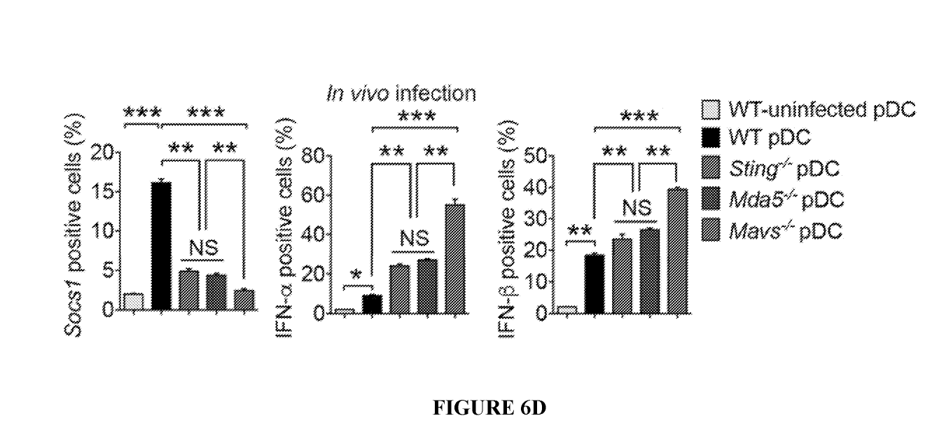

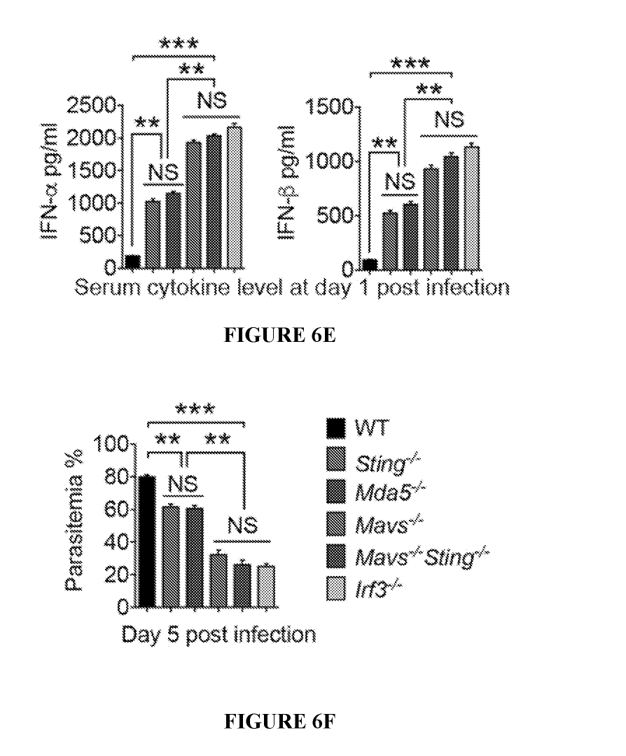

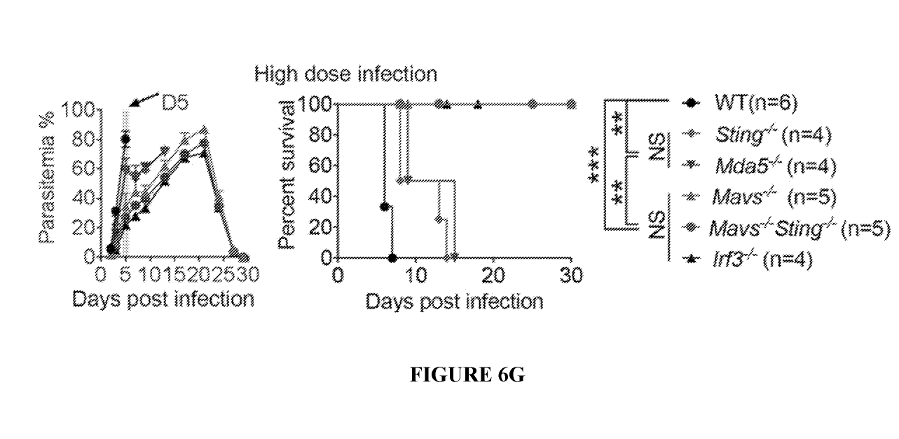

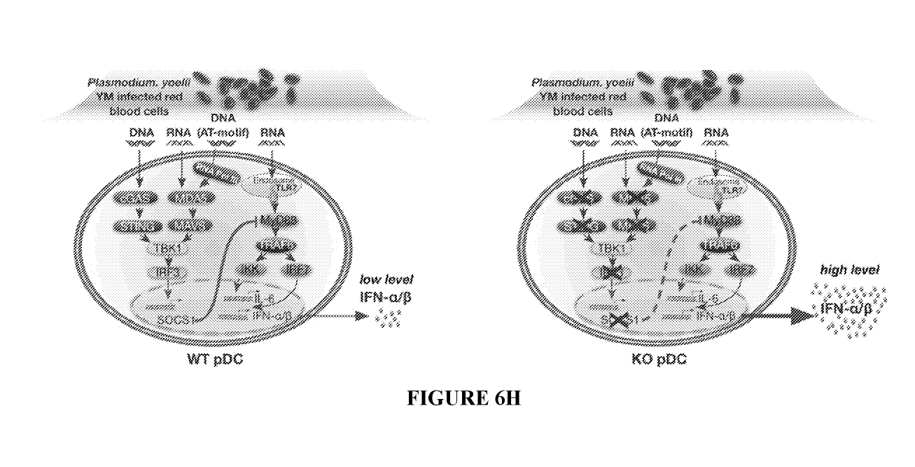

Next, experiments were conducted to determine the role of SOCS1 in inhibiting MyD88-mediated type I IFN production and protective immunity in vivo after YM infection. Due to neonatal mortality (mice die within 3 weeks after birth) and the complex inflammatory pathology of Socs1^{-/-} mice, the in vivo role of SOCS1 in response to malaria infection could not be addressed in these mice. Hence, Socs1 gene was first knocked down in WT mice with Socs1-specific siRNA (FIG. 5g). As expected, Socs1 KD increased IFN-γ production through its inhibitory effect on JAK1. Increased serum levels of IFN-α and IFN-β were observed compared with WT mice treated with scrambled siRNA after YM infection (FIG. 5h). Most importantly, knockdown of Socs1 resulted in reduction of parasitemia and increased survival of WT mice after YM infection compared with scrambled siRNA-treated WT mice (FIG. 5i). To further confirm these observations, Socs1 gene was knocked out in WT bone marrow cells using the CRISPR/Cas9 system, and then chimeric mice were generated (FIG. 14d-6e). Socs1 KO chimeric mice produced high levels of IFN-α and IFN-β, reduced parasitemia and increased survival in response to YM infection (FIG. 14f-14g). These results suggest that KD or KO of Socs1 relieves its inhibition of MyD88-dependent type I IFN signaling pathway, thus leading to the control of parasitemia and reduction in host mortality.

SOCS1-Mediated Inhibition of Type I IFN Signaling is pDC-Specific and MyD88-Dependent

To provide direct evidence that SOCS1-mediated inhibition of MyD88-dependent type I IFN signaling is pDC-specific, we performed Socs1 KD in WT mice at 24 h before infection with or without pDC depletion using anti-mPDCA-1. We showed that the high serum levels of IFN-α and IFN-β observed in Socs1-silenced WT mice were abolished upon pDC depletion (FIG. 6a). Consistently, pDC depletion in Socs1-silenced WT mice also increased parasitemia and host mortality after infection compared with Socs1-silenced WT mice without pDC depletion (FIG. 6b). These results suggest that SOCS1-mediated inhibition of MyD88-dependent type I IFN signaling is pDC-specific.

Because SOCS1 can inhibit MyD88-dependent type I IFN signaling (as presented in this study) and JAK1 in STAT signaling (a downstream IFN-stimulated signaling pathway for IFN-γ production) (FIG. 14h), experiments were conducted to determine whether SOCS1-mediated type I IFN signaling inhibition is MyD88-dependent. As expected, WT mice with Socs1 KD reduced parasitemia and increased survival after YM infection. By contrast, Myd88^{-/-} mice with Socs1 KD did not show reduced parasitemia or increased survival compared with those treated with scrambled siRNA (FIG. 6c). Consistently, no IFN-α and IFN-β were not detected in Myd88^{-/-} mice regardless of Socs1 KD, suggesting that SOCS1-mediated type I IFN signaling inhibition is MyD88-dependent. Interestingly, IFN-γ production was markedly increased in in Myd88^{-/-} mice with Socs1 KD, compared with Myd88^{-/-} mice with scrambled siRNA (FIG. 14i), suggesting that SOCS1 regulates IFN-γ production mainly through its inhibitory effect on Jak1. Taken together, these results suggest that SOCS1, which is induced by the STING/MAVS pathway in response to YM infection, mainly inhibits MyD88-dependent type I IFN signaling and host survival.

SOCS1 Induction and its Inhibition of MyD88-Dependent Type I IFN Signaling is Finely Tuned in Different KO Mice

Because DNA sensor/adaptor (cGAS-STING)- and RNA sensor/adaptor (MDA5-MAVS)-induced signaling pathways converge at TBK to activate IRF3-mediated type I IFN signaling for SOCS1 upregulation, a key question is whether ablation of one or two of these molecules (STING, MDA5, MAVS and IRF3) in IRF3-mediated type I IFN signaling has equal effect on SOCS1 expression, and subsequently on IFN-α/IFN-β production by MyD88-dependent type I IFN signaling pathway. To address this issue, pDCs were isolated from YM-infected WT and different KO mice (Sting^{-/-}, Mda5^{-/-}, and Mavs^{-/-}) mice at 18 h post YM infection and determined expression levels of SOCS1, IFN-α and IFN-β. 17% of YM-infected WT pDCs expressed SOCS1, but only 3.8% of Sting^{-/-} pDCs, 2.8% of Mda5^{-/-} pDCs and 2% of Mavs^{-/-} pDCs expressed SOCS1 (FIG. 6d). Conversely, only 14% of pDCs isolated from in WT mice expressed IFN-α and IFN-β, compared with 25% of pDCs from Mda5^{-/-} and Sting^{-/-} mice and 40% of pDCs from Mavs^{-/-} mice that expressed IFN-α and IFN-β (FIG. 6d). Thus, expression levels of SOCS1 are finely tuned in different KO mice and inversely correlated with IFN-α and IFN-β production in WT, Sting^{-/-} Mda5^{-/-}, and Mavs^{-/-} pDCs. To further determine whether the fine-tuning of MyD88-IRF7-mediated type I IFN signaling by STING/MAVS-induced SOCS1 expression is correlated with protective immunity in vivo, WT, Sting^{-/-}, Mda5^{-/-}, Mavs^{-/-}, Mavs^{-/-}:Sting^{-/-} DKO, and Irf3^{-/-} mice were infected with 1×10⁷ iRBCs (a 10-fold higher dose than that used in other experiments). IFN-α and IFN-β production in Mavs^{-/-}, Mavs^{-/-}:Sting^{-/-}, and Irf3^{-/-} mice was significantly higher than those in Mda5^{-/-} and Sting^{-/-} mice, which produced much more IFN-α and IFN-β than WT mice (FIG. 6e). The levels of IFN-α and IFN-β production were inversely correlated with parasitemias in different KO mice (FIG. 6e-6f). More importantly, Mavs^{-/-}, Mavs^{-/-}:Sting^{-/-}, and Irf3^{-/-} mice were completely protected and capable of clearing malaria after 4 weeks; by contrast, Mda5^{-/-} and Sting^{-/-} mice failed to control malaria infection and died at day 15 post infection, even though these KO mice provided better protection than WT mice (FIG. 6g). Thus, these results provide clear evidence that SOCS1 expression levels are finely tuned by activation signaling from cGAS-STING- and MDA5-MAVS-induced IRF3-dependent type I IFN pathway, and are inversely correlated with IFN-α and IFN-β production through MyD88-dependent type I IFN signaling in pDCs, as well as with their protective immune responses against YM infection. These new findings may facilitate the efforts to determine the underlying mechanisms for strain-specific and -shared immune responses for malaria strains, which undoubtedly provide new avenues and therapeutic targets for the development of safe and effective malaria vaccines. These results may facilitate the efforts to determine the underlying mechanisms for malaria strain-specific and -shared type I IFN signaling responses, which undoubtedly provide new avenues and therapeutic targets for the development of safe and effective malaria vaccines.

Plasmodium. Yoelii YM Infection Inhibits Melanoma Growth by Activating Type I IFN Signaling in pDCs

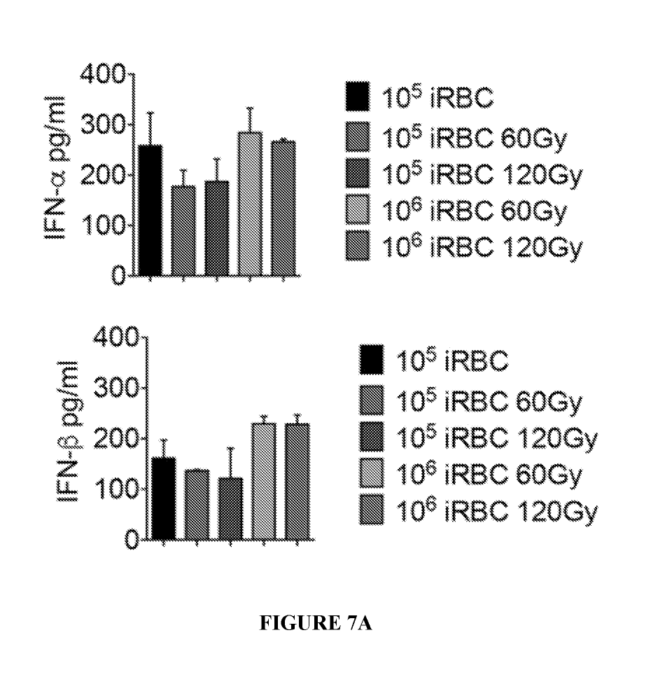

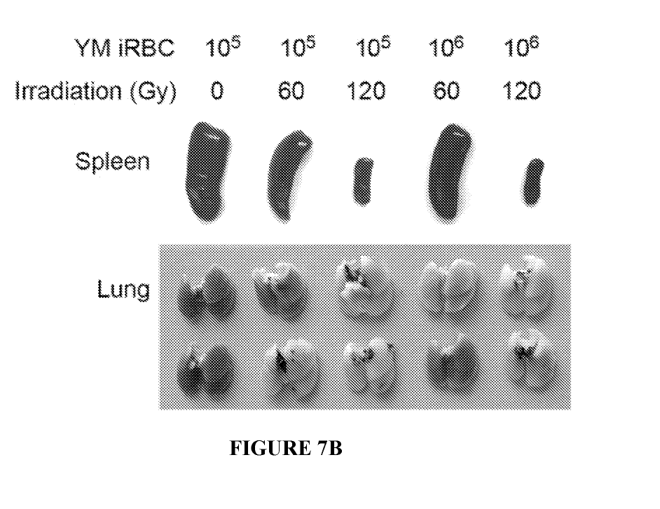

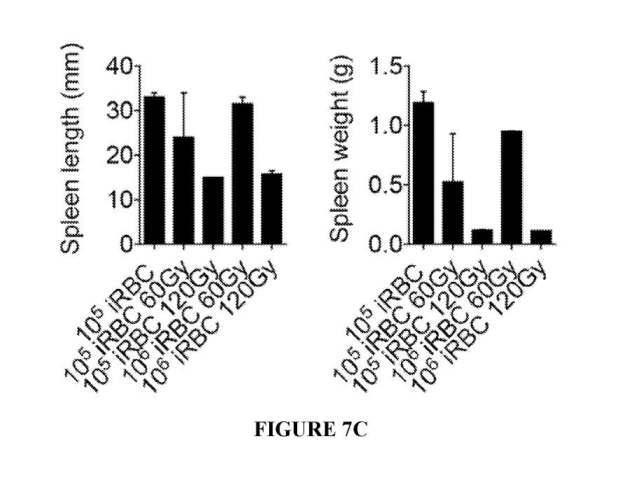

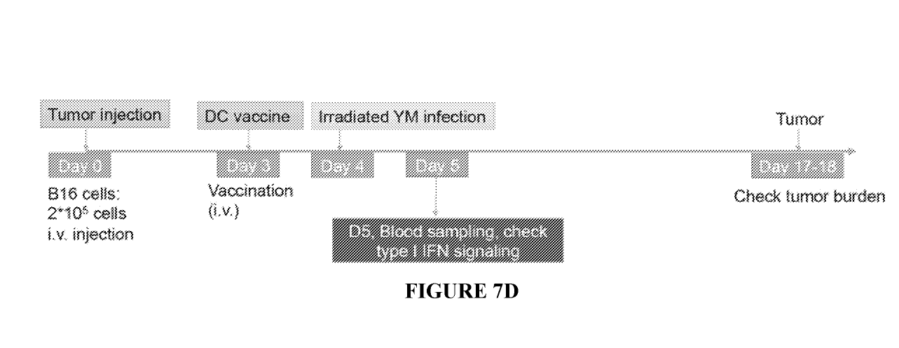

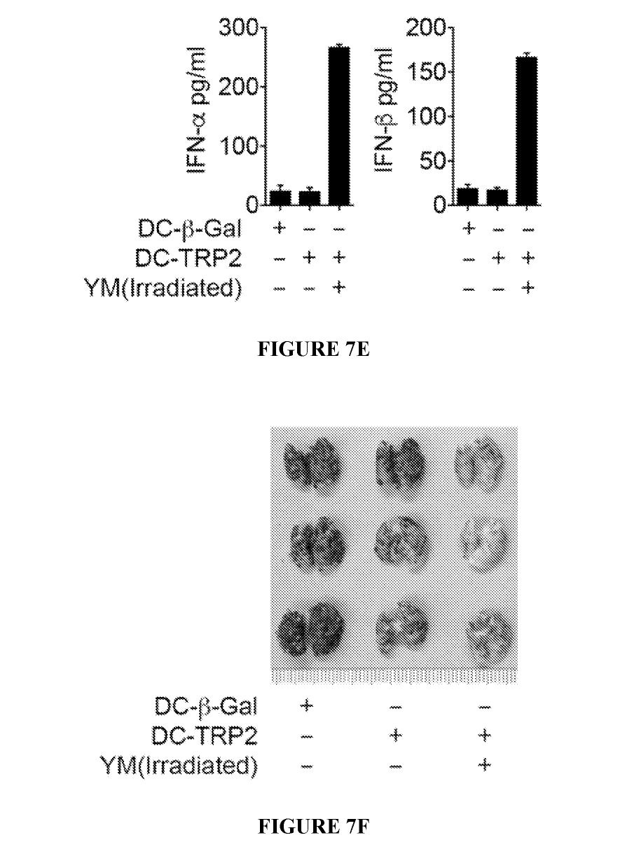

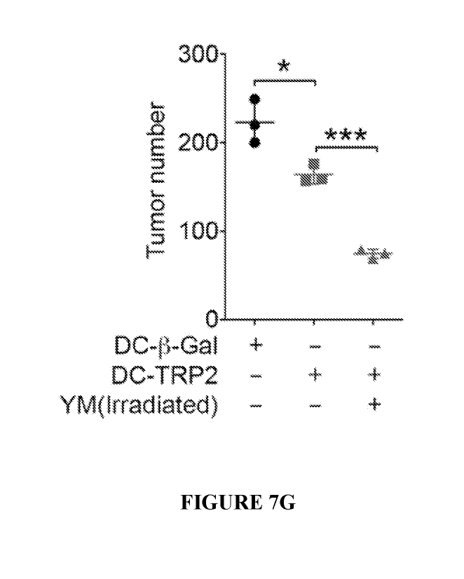

Because lethal Plasmodium. yoelii YM infection causes rodent death due to high parasitemia and rupture of red blood cells, experiments were conducted to determine whether irradiated P. yoelii YM for the malaria infection would avoid such problems. By detecting the spleen and lung at day 15 after YM infection, 120 Gy of 10⁶ YM iRBCs was found to be the appropriate dosage for the malaria infection by activating type I IFN in pDCs (FIG. 7a), but did not cause splenomegaly (FIGS. 1B and 1C) and damage of the lung tissue (FIG. 1B). Next experiments were conducted to determine whether malaria infection could enhance anti-tumor immunity induced by DC/TRP-2 vaccine. C57BL6 mice were injected with B16 melanoma tumor cells at day 0, vaccinated with DC/TRP2 vaccine at day 2 after and then infected with irradiated P. yoelii YM at day 4. Blood samples were collected before and after YM infection for serum cytokine level analysis. Mice were sacrificed on day 18, and lung tissues were collected for determining the number of lung metastasis. Irradiated P. yoelii YM effectively activated IFN-α and IFN-β in TRP2-vaccine treated mice (FIG. 7d, 7e), and markedly reduced lung metastasis in B16 tumor mode, compared with DC/TRP-2 treatment (FIG. 7f, 7g). These results suggest that activation of type I IFN signaling in pDCs by irradiated YM infection enhances antitumor immunity.

Robust Production of Type I IFN in Mavs^{-/-} Mice Generates More Potent Antitumor Immunity than in WT Mice

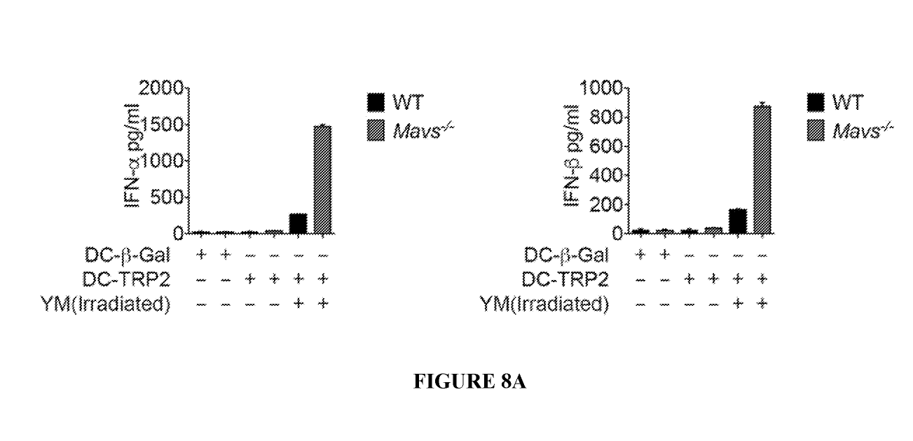

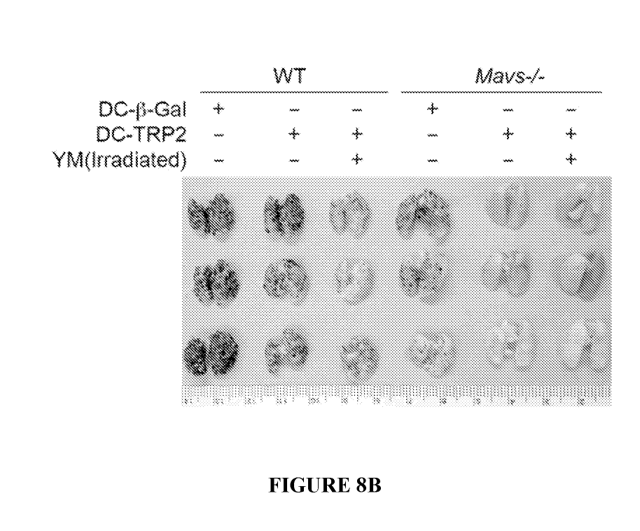

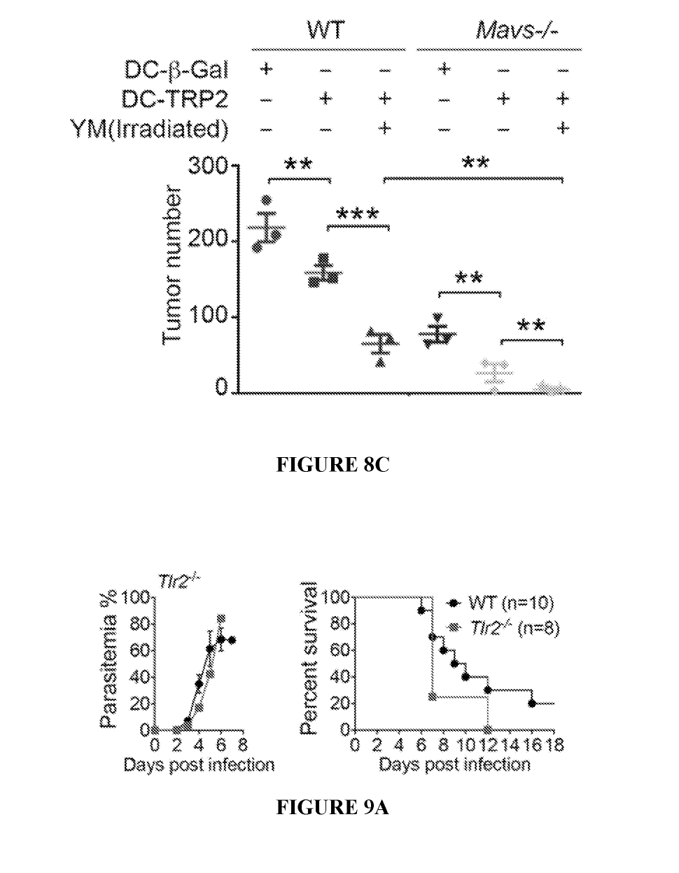

Since IFN-α and IFN-β production through MyD88-dependent type I IFN signaling in pDCs in Mavs^{-/-}, Sting^{-/-} and Mda5^{-/-} mice was significantly higher than WT mice, experiments were conducted to determine whether Mavs^{-/-} mice would generate more potent antitumor immunity than WT mice. As expected, B16-bearing Mavs^{-/-} mice produced significantly higher amounts of type I IFN in serum than WT mice at 24 h after P. yoelii YM infection (FIG. 8a). Importantly, Mavs^{-/-} mice markedly reduced the number of B16 lung metastasis compared with WT mice with or without irradiated P. yoelii YM infection. However, the combination of DC/TRP-2 vaccination with irradiated P. yoelii YM infection almost completely eliminated tumor cells and generated much stronger antitumor immunity after DC/TRP-2 vaccination Mavs^{-/-} mice than WT mice (FIGS. 8b and 8c). These results suggest that P. yoelii YM infection could markedly enhance DC/TRP2-induced antitumor immunity by targeting MyD88-dependent type I IFN signaling pathway in pDCs in Mavs^{-/-} mice. Taken together, these findings not only identify a previously unrecognized utility of malaria infection in enhancing antitumor immunity by manipulating type I IFN signaling pathways, but also provide new strategies for the development of therapeutic vaccines against cancer and other infectious diseases.

Discussion

This study demonstrates for the first time that cGAS functions as a DNA sensor for recognizing malaria gDNA and triggers STING-mediated type I IFN signaling. Interestingly, Sting-deficient mice were more resistant to YM infection than were cGAS-deficient mice, suggesting that other DNA sensors might function in recognizing malarial genomic DNA. For RNA sensing, MDA5, but not RIG-I, is responsible for detecting malaria RNA and triggers MAVS-mediated type I IFN signaling. It is known that RIG-I recognizes RNA with a triphosphate (PPP) moiety and 5′ blunt-ended 20 nucleotides, whereas MDA5 recognizes long dsRNA (1-2 kb) (Goubau, D., et al. Immunity 38:855-869 (2013)). These structural and length requirements of dsRNA may explain why malaria RNA interacts with MDA5, but not with RIG-I, to trigger downstream MAVS-dependent type I IFN signaling. Although cGAS-STING/MDA5-MAVS-mediated type I IFN signaling is important and operates in all cell types, including macrophages and cDCs, YM malaria 18S rRNA was detected and IFN-β and SOCS1 expression was not observed in macrophages and cDCs within the first 18-24 h post infection. This suggests that macrophages and cDCs may not be involved the production of IFN-α and IFN-β in the early stage of YM infection.

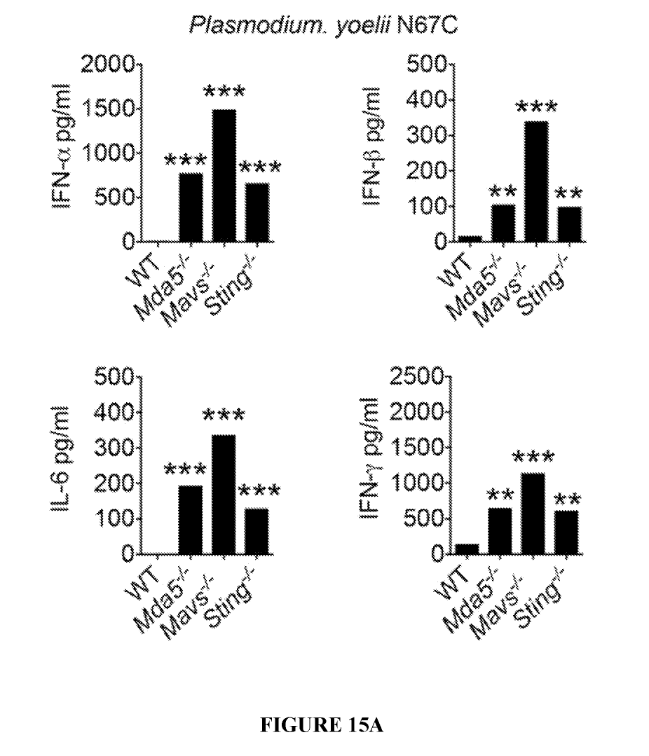

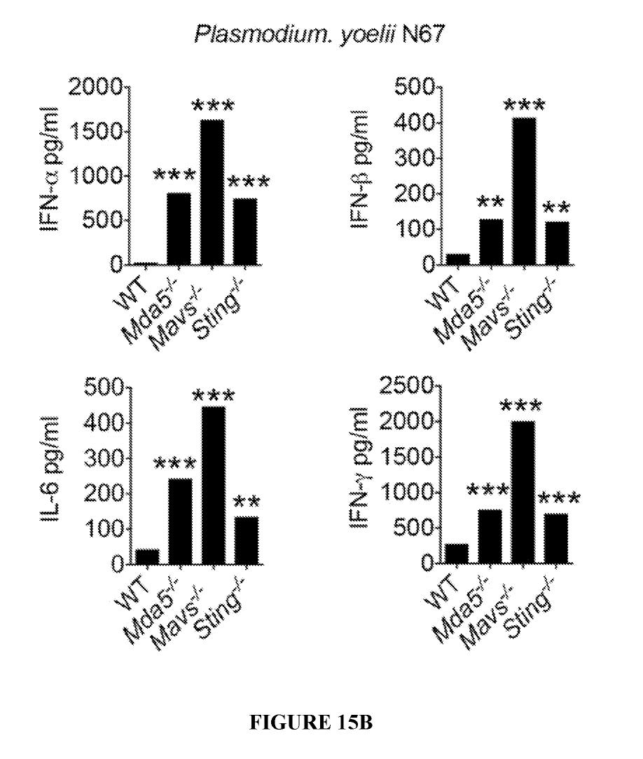

The fact that there were 10-fold higher serum levels of IFN-α and IFN-β in Mda5^{-/-}, Mavs^{-/-}, Sting^{-/-}, or cGAS^{-/-} mice than those in WT mice after YM infection suggests that alternative cell types or signaling pathways must be activated to account for the robust production of IFN-α and IFN-β at 24 h post infection. There are several lines of evidence to support this conclusion: 1) Mda5:Myd88 DKO, Mavs:Myd88 DKO, or Sting:Myd8 DKO mice failed to produce any IFN-α and IFN-β and become susceptible to YM infection, compared with Mda5^{-/-}, Mavs^{-/-} and Sting^{-/-} mice; 2) depletion of pDCs by specific antibody or genetic ablation markedly reduced the production of IFN-α and IFN-β, suggesting that pDCs are the major sources for production of these cytokines. Interestingly, the high serum levels of cytokines were also observed in Mda5^{-/-}, Mavs^{-/-}, Sting^{-/-} mice in response to different malaria strains (N67 and N67C) (FIG. 15), even though diseases pathogenesis differs from YM. These results provide the first evidence that MyD88-dependent type I IFN signaling in pDCs plays a critical role in the early production of IFN-α/β and protection against malaria infection.

TLR7, but not TLR9, is shown to be essential for IFN-α and IFN-β production in pDCs. Like Myd88 deficiency, Tlr7^{-/-}, Irf7^{-/-}, Sting^{-/-}:Tlr7^{-/-}, or Irf3^{-/-}:Irf7^{-/-} mice failed to produce IFN-α and IFN-β and had increased sensitivity to YM infection. These findings suggest that YM RNA activates TLR7 and recruits the downstream adaptor MyD88 to trigger IRF7-mediated type I IFN signaling pathway. However, mice deficient with Sting, Tbk1, Irf3/Irf7, or Ifnar expression are resistant to Plasmodium berghei ANKA infection (Sharma, S. et al. Immunity 35:194-207 (2011); Haque, A. et al. J Clin Invest 124:2483-2496 (2014)). Thus, the requirement or opposing effects of IRF3- and/or IRF7-mediated type I IFN signaling may be dependent upon distinct pathogenesis, cell tropism (targeting different cell types during infection), and the level and timing of type I IFN production of different malaria strains (Wu, J. et al. Proc Natl Acad Sci USA 111:E511-520 (2014); Gun, S. Y., et al. Mediators of inflammation 2014:243713 (2014)). In the case of YM infection, TLR7 and MyD88 are essential for the robust production of IFN-α and IFN-β for resistance to YM infection in Mda5^{-/-}, Mavs^{-/-}, and Sting^{-/-} mice. Activation of TLR7-MyD88-dependent type I IFN signaling might be blocked by negative regulators activated by STING- or MAVS-mediated type I IFN signaling in WT mice. Indeed, SOCS1 was identified as a key negative regulator, which interacted with MyD88 and inhibited MyD88-dependent type I IFN signaling. Importantly, expression of Socs1, but not Socs3, Ship1, or Ship2, was induced in pDCs by STING/MAVS-mediated type I IFN signaling. Interestingly, a recent study shows a role for STING in negatively regulating inflammatory responses of macrophages in systemic lupus erythematosus (SLE) through upregulation of A20, SOCS1, and SOCS3 (Sharma, S. et al. Proc Natl Acad Sci USA 112:E710-717 (2015)), but its role in MyD88-dependent type I IFN signaling is not known in this disease model. Inactivation of Mda5, Sting, or Mavs markedly reduced Socs1 expression in vitro and in vivo and increased IFN-α and IFN-β mRNA and protein expression. Most strikingly, KD or KO of Socs1, using siRNA or the CPRISPR/Cas9 system in YM-infected WT mice, significantly reduced parasitemia and host mortality, compared with YM-infected WT mice treated with scrambled siRNA. The predominant role of SOCS1 is to regulate MyD88-dependent type I IFN signaling in pDCs, because the observed increase in IFN-α and IFN-β expression after siRNA-mediated Socs1 KD disappeared when pDCs were depleted using anti-mPDCA-1, suggesting that Socs1 KD mainly affects MyD88-dependent type I IFN signaling in pDCs.

Collectively, this study shows that pDCs are the major cells sensing and detecting lethal YM in the first 24 h post infection through cGAS-STING and MDA5-MAVS-induced IRF3 and TLR7-MyD88-induced IRF7 type IFN signaling pathways (FIG. 6h). Activation of cGAS-STING and MDA5-MAVS triggers IRF3-mediated type I IFN signaling in pDCs, which produce low amounts of IFN-β and activate negative regulators, such as SOCS1 in WT pDCs. Although the constitutive expression of TLR7 in pDCs allows for the recruitment of MyD88, in response to YM infection, which triggers IRF7-dependent type I IFN signaling, SOCS1 upregulation by cGAS-STING and MAD5-MAVS triggers IRF3-mediated type I IFN signaling. This inhibits MyD88-dependent type I IFN signaling pathway, which produces more than 100 fold higher cytokine levels than those produced by STING/MAVS-mediated IRF3-dependent type I IFN signaling pathway. By contrast, SOCS1 expression is markedly reduced Mda5^{-/-}, Mavs^{-/-}, and Sting^{-/-} pDCs, allowing the activation of MyD88-dependent type I IFN signaling. Thus, lethal YM infection activates two distinct type I IFN signaling pathways in pDCs. YM takes advantage of the cross-inhibition of TLR7-MyD88 mediated type I IFN signaling by cGAS-STING/MDA5-MAVS-mediated type I IFN through upregulation of Socs1 in WT mice to avoid innate immune response. KO or KD of genes such as Socs1 or those in the STING/MAVS-mediated type I IFN signaling pathway relieves SOCS1 suppressive effects on TLR7-MyD88-mediated type I IFN signaling and permits robust production of IFN-α and IFN-β for generating innate and adaptive immunity against YM. Most importantly, robust production of IFN-α and IFN-β through MyD88-dependent type I signaling pathway in pDCs by blockade of cGAS-STING and MAD5-MAVS triggers IRF3-mediated type I IFN signaling or SOCS1 expression markedly enhance antitumor immunity in a B16 tumor model. Thus, these findings not only identify a previously unrecognized regulatory mechanism between different type I IFN signaling pathways in pDCs, but also provide novel strategies for development of therapeutic vaccines against infectious diseases and cancer.

Methods

Malaria Parasites and Mice.

The parasite Plasmodium yoelii YM, N67C and N67 have been previously described (Li, J. et al. Proc Natl Acad Sci USA 108:E374-382 (2011)). For plasmodium infection, 0.2 (low) or 2×10⁶ iRBCs (otherwise, indicated specifically in the figure legend) suspended in 200 μl PBS from the donor mice were intraperitoneally injected into experimental mice. A higher parasite dose (1×10⁷ iRBCs) was used in some experiments. Parasitemias were monitored daily by examination of Giemsa-stained thin tail blood smears. Female mice C57BL/6 (WT), Mavs^{-/-}, Mda5^{-/-}, Myd88^{-/-}, Sting^{-/-}, Trif^{-/-}, BDCA2-DTR, Tlr2^{-/-}, Tlr3^{-/-}, and Tlr4^{-/-} mice were purchased from The Jackson Laboratory. Tlr9^{-/-} mice, Tlr7^{-/-}, Rig-I^{-/-} mice, Irf3^{-/-}:Irf7^{-/-} mice, and cGAS^{-/-} mice were obtained. All mouse-related procedures were performed according to experimental protocols approved by the Animal Care and Welfare Committee at Houston Methodist Research Institute and in accordance with NIH-approved animal study protocol LMVR-11E.

Antibody Treatments.

To deplete pDCs, pDC-depleting functional-grade mAb (anti-mPDCA-1 IgG, clone JF05-1C2.4.1) and the corresponding isotype control IgG, which served as control, were purchased from Miltenyi Biotec (Auburn, Calif.). Two intraperitoneal injections of antibody (250 μg/mouse) per mouse were administered 12 h prior and after YM infection.

Isolation and Preparation of Plasmodium gDNA and RNA.

Parasite-infected mice blood was collected in saline solution and filtered to deplete white blood cells. Parasites were spun down after RBC lysis buffer treatment, and lysate incubated with buffer A (150 mM NaCl, 25 mM EDTA, 10% SDS and protein kinase) overnight. gDNAs were isolated using phenol/chloroform, and RNAs were isolated using TRIzol reagent (Invitrogen).

Isolation and Purification of Immune Cell Populations.

Bone marrow cells were isolated from the tibia and femur and cultured in RPMI1640 medium with 10% FBS, 1% penicillin-streptomycin, 55 μM β-mercaptoethanol, and 10% L929 conditioned media, containing macrophage-colony stimulating factor (M-CSF) for 5 days for BMDMs, 20 ng/ml murine GM-CSF and 10 ng/ml IL-4 for 6-8 days for cDCs. pDCs were generated from bone marrow in the culture medium containing 200 ng/ml Flt3L and further purified by FACS analysis and were gated on the CD11b⁻B220⁺CD11c⁺ cell population. For specific cell isolation from splenocytes, pDCs were isolated using anti-mPDCA-1 microbeads from Miltenyi Biotec (Auburn, Calif.). After pDC isolation, macrophages were isolated with CD11b microbeads from Miltenyi Biotec, cDCs were isolated with mouse CD11cPE labeling and followed by PE selection cocktail from STEMCELL technologies, following the manufacturer's protocol.

Flow Cytometry and ELISA.

Mouse splenocytes were prepared from spleens cut into small fragments and digested with collagenase D (1 mg/ml, Sigma) and DNAse I (20 μg/ml, Sigma) in RPMI-1640 medium for 30 min. Single cells were collected and cultured for 5 h in the presence of Golgi Stop solution (BD Biosciences), harvested for the surface molecular staining with the appropriate fluorescent mAbs (anti-CD11b, anti-F4/80, anti-CD11c, and anti-B220), and then fixed, permeabilized (FIX AND PERM, Invitrogen) and stained with fluorescein-labeled cytokine-specific mAbs (FITC-anti-mouse IFN-α, FITC-anti-mouse IFN-β were purchased from PBL; anti-Socs-1-mAb-Biotin was purchased from MBL) following the manufacturer's instructions. Appropriate fluorescein-conjugated, isotype-matched mAbs were used as negative controls. Cells were analyzed using the BD FACS Aria II. For ELISA, mouse serum or cell supernatants were collected at the indicated time after infection or stimulation and subjected to analysis with commercial ELISA kits for mouse IFN-α, IFN-β, (PBL Biomedical Laboratories) or IFN-γ (eBioscience), following the manufacturer's instructions.

RNAi-Mediated Knockdown in Mice.

In Vivo Ready siRNAs were mixed with Invivofectamine 3.0 liposomes (Invitrogen) following the manufacturer's instructions and injected intravenously in a volume of 100 μl at a dose of 5 mg/kg. Mice were infected with P. yoelii YM (0.5×10⁶ iRBCs) 48 h after siRNA treatment.

RNA Preparation and qPCR.

Total RNA was harvested from splenic tissue or stimulated cells using the TRIzol reagent (Invitrogen), and the complimentary cDNA was generated using reverse transcriptase III (Invitrogen). Real-time PCR was carried out using the ABI Prism 7000 analyzer (Applied Biosystems) using the SYBR GreenER qPCR Super Mix Universal (Invitrogen) and specific primers.

Isolation and Purification of pDCs and Transfection with RNAi.

Bone marrow cells were isolated and cultured with 200 ng/ml Flt3L for 9 days and isolated by FACS analysis for pDCs (CD11b⁻B220⁺CD11c⁺). siRNA oligonucleotides specific for cGAS, Dai, Ddx41, Ifi16, Socs1, Socs3, Ship1, Ship2, and control (scrambled siRNA) were purchased from Invitrogen and nucleotransfected into RAW264.7 cells or pDCs cells for 48 h using Amaxa nucleofector kit, following the manufacturer's instructions (Lonza). Next, cells were stimulated with 1 μg plasmodium gDNA or RNA, using Lipofectamine 2000 reagent (Invitrogen), at the indicated time points. Supernatants were tested for cytokine production by ELISA, and RNAs were extracted for qPCR assay.

Mouse Bone Marrow Transplant.

Total bone marrow was isolated from the femurs and tibias from 8-week-old female C57BL/6 mice. Bone marrow was subjected to erythrocyte lysis, and then transduced with concentrated Socs1-specific or scrambled sgRNA lentiviral supernatant in the presence of 2 μg/ml polybrene. At 24 h post transduction, cells were collected and intravenously injected into lethally irradiated (950 cGy) 6-week-old female C57BL/6 mice.

Diphtheria Toxin (DT) Treatment.

Deficient (Mda5^{-/-}, Mavs^{-/-}, and Sting^{-/-}) mice were crossed with BDCA2-DTR transgenic mice and treated with diphtheria toxin (DT, Sigma-Aldrich) intraperitoneally (i.p.) at a dose of 100-120 ng/mouse. pDCs were depleted by DT injection at 1 day before and 1, 3, and 5 days after P. yoelii YM infection.

DC/TRP-2 Vaccine Preparation.

Bone marrow cells were isolated from the tibia and femur and cultured in RPMI1640 medium with 10% FBS, 1% penicillin-streptomycin, 55 μM β-mercaptoethanol and 10% L929 conditioned media, containing 20 ng/ml murine GM-CSF and 10 ng/ml IL-4 for 6 days. TRP2 or β-Gal control peptide was incubated with DC for 4 h. DC were washed twice after incubation, and injected into B16 tumor-bearing mice intravenously.

Statistical Analysis.

All analyses were performed using GraphPad Prism version 5.0 (GraphPad Software, La Jolla, Calif.). Data are presented as means±s.d., unless otherwise stated. Statistical significance of differences between two groups was assessed by unpaired Student t tests and a p value of <0.05 was considered significant.

### Example 2: Type I IFN-Induced Immunity and its Utility for Vaccine Development Against Malaria and Other Infectious Diseases

Disclosed in Example 1 is a new regulatory mechanism of type I IFN signaling pathways in plasmacytoid dendritic cells (pDCs). However, how the early robust production of type I IFN in pDCs in different knockout (KO) mice (Sting^{-/-}, Mda5^{-/-}, and Mavs^{-/-}) is linked to subsequent development of innate and adaptive immunity was not clear. This Examples shows that early robust IFN-α/β production by pDCs in KO mice during the first 24 h of P. yoelii YM infection subsequently activates cDCs and macrophages through interferon-α/β receptor (IFNAR)-mediated downstream Jak1/Stat signaling pathways, which, in turn, induce T- and B cell-mediated adaptive immune responses. Importantly, adaptive immunity induced by P. yoelii YM infection in Mavs^{-/-} mice could protect the host from the second challenge of the same or different malaria strains. Thus, these findings have identified a stage-specific function of pDCs, macrophages, cDCs, and T/B cells for generating innate and adaptive immunity against lethal malaria infection and provided new strategies to develop therapeutic malaria vaccines.

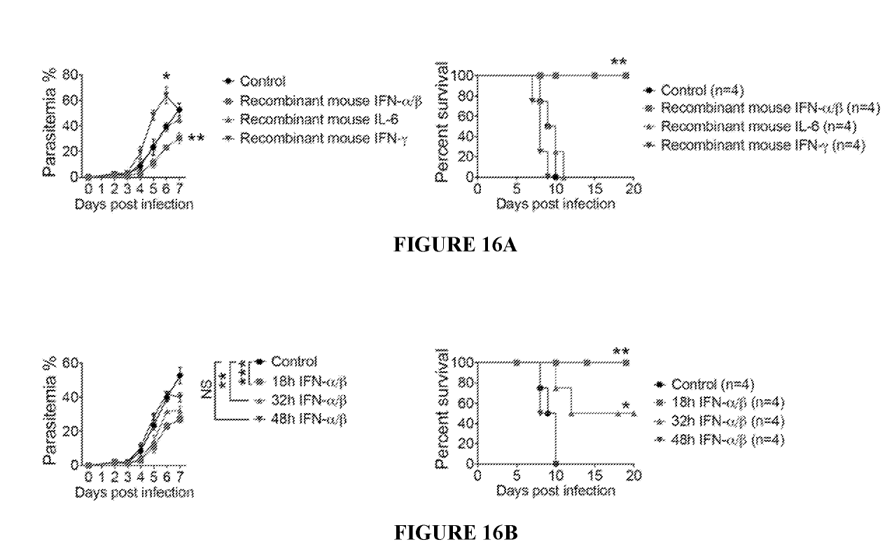

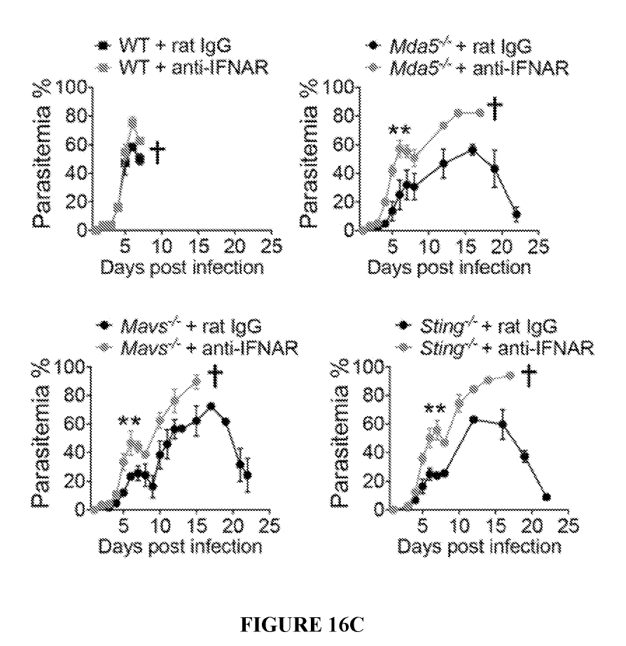

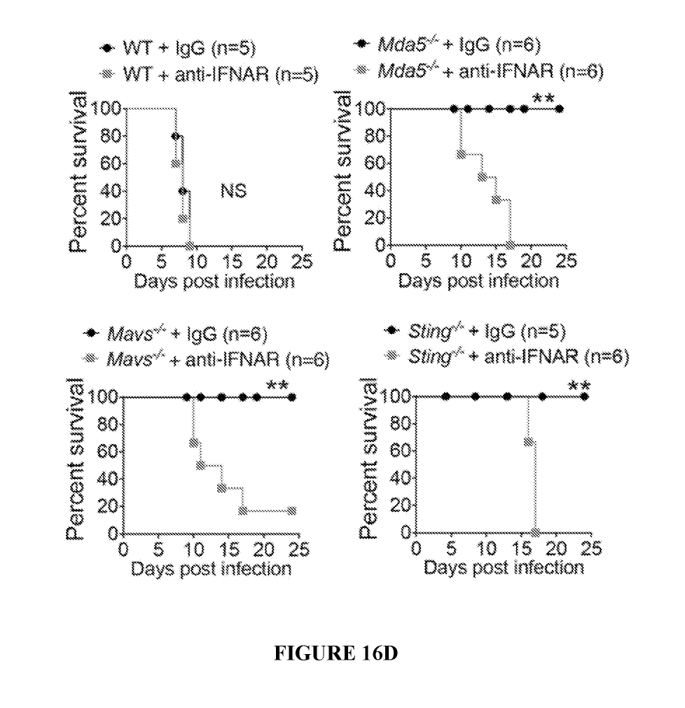

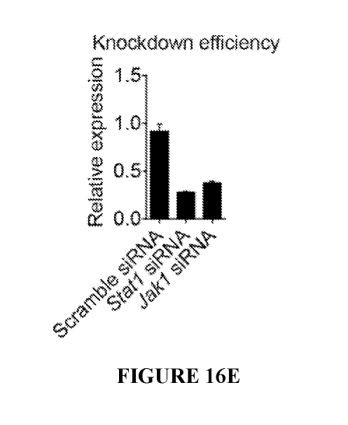

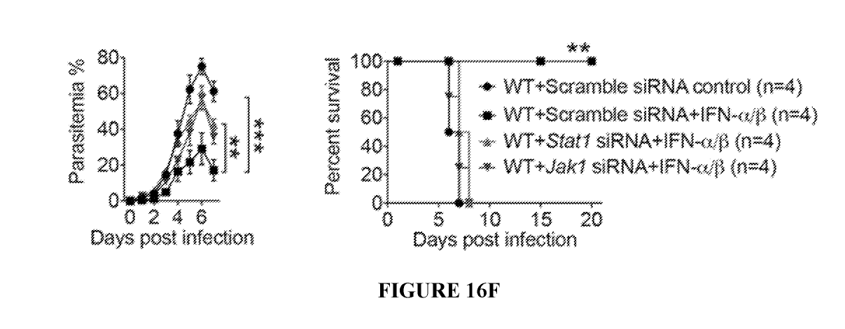

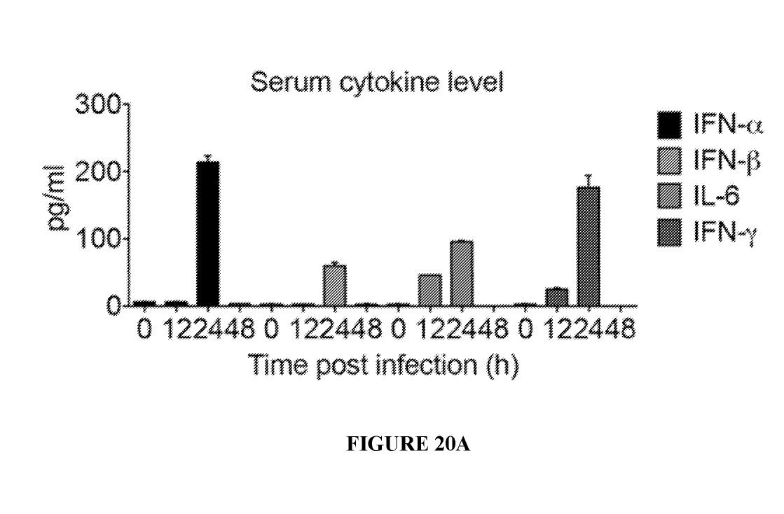

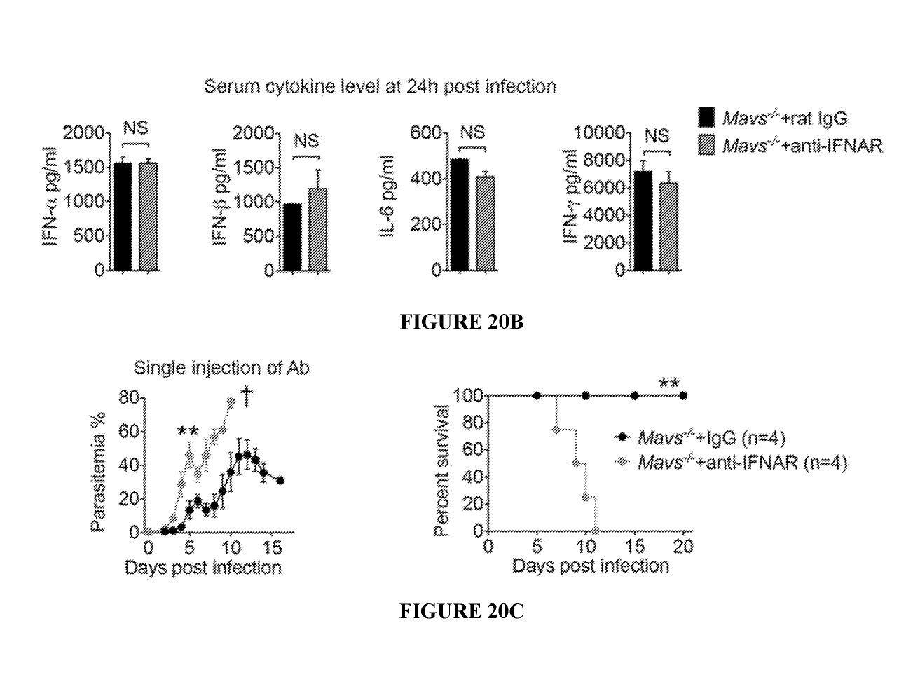

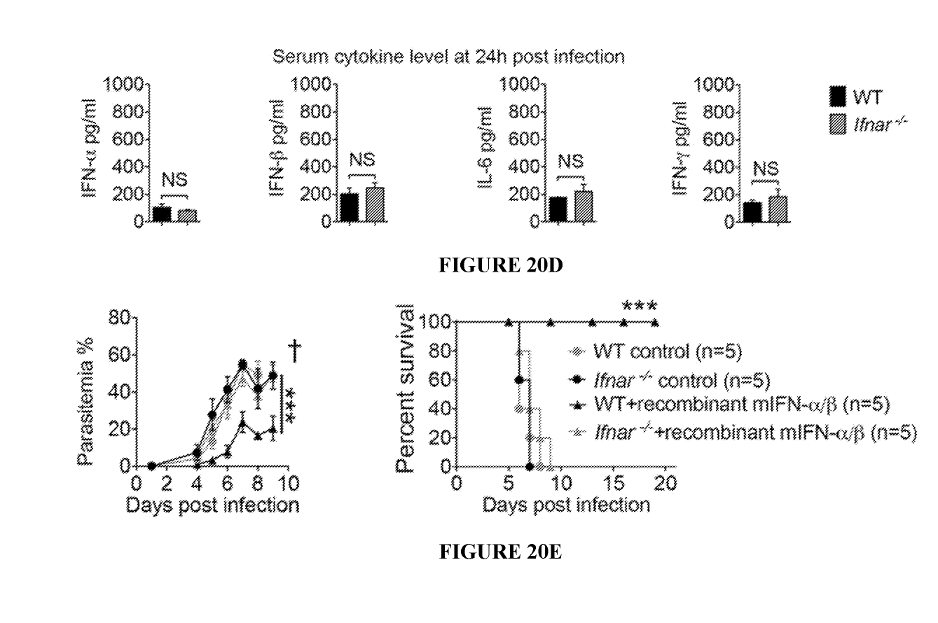

The above study showed that cGAS-STING and MDA5-MAVS-induced type I IFN signaling inhibits TLR-MyD88-IRF7-mediated type I IFN signaling pathway in plasmacytoid dendritic cells (pDCs) through upregulation of SOCS1 in WT mice (see Examples 1). In addition to the increased serum levels of IFN-α and IFN-β, the serum levels of IFN-γ and IL-6 were much higher in Mda5^{-/-}, Mavs^{-/-}, and Sting^{-/-} mice than WT mice after lethal Plasmodium yoelii YM infection (FIG. 20a). To determine which cytokines are responsible for generating protective immunity against P. yoelii YM infection, WT mice were injected with exogenous recombinant IFN-α/β, IFN-γ, or IL-6, and their ability to inhibit parasitemia and reduce host mortality after YM infection was tested. WT mice treated with IFN-α and IFN-β after YM infection had markedly decreased parasitemia and host mortality, but treatment with IL-6 or IFN-γ failed to do so (FIG. 16a). Notably, treatment of WT mice with IFN-γ actually slightly increased parasitemias and sensitivity to YM infection compared with untreated mice. These data indicated that IFN-α/β are required for the protective immunity against YM infection. Since the production of IFN-α and IFN-β in serum peaked at 24 h after YM infection and then disappeared, we asked whether the timing of cytokine treatment was important for protective immunity. We performed similar experiments with cytokine injections at different time points and found that levels of IFN-α- and IFN-β-mediated protection (mouse survival) were the highest at 18 h after YM infection, intermediate at 32 h, and the lowest at 48 h (FIG. 16b). These results suggest that timing of IFN-α/β administration or in vivo production is critical for inducing protective immunity against YM infection.

Next, experiments were conducted to determine the molecular mechanisms of how an early robust burst of IFN-α/β can generate potent innate and adaptive immune responses, which inhibit parasitemia and host mortality and eventually clear malaria infection. It was hypothesized that IFN-α/β may initiate a cascade of signaling events to generate potent immune responses. As the first step, experiments were conducted to determine whether IFN-α/β-mediated activation of IFNAR and downstream Stat/Jak signaling pathway are required for controlling parasitemia and host mortality. Type I IFN signaling was blocked in Mda5^{-/-}, Mavs^{-/-}, and Sting^{-/-} mice by administering anti-IFNAR (interferon-α/β receptor) antibody. Although anti-IFNAR treatment did not have much effect on the production of IFN-α, IFN-β, IFN-γ, and IL-6 in Mavs^{-/-} mice (FIG. 20b), the Mda5^{-/-}, Mavs^{-/-}, and Sting^{-/-} mice treated with anti-IFNAR antibody with a single injection at one day before infection (i.e. day −1) or four injections at days −1, 2, 4, and 6 showed increased parasitemias and died around day 15 (FIG. 20c, FIG. 16c-16d). These results suggest that IFN-α/β-mediated downstream signaling pathway is critical in controlling parasitemia and host mortality. To further test whether IFN-α/β-induced immunity requires IFNAR and its downstream signaling, Ifnar^{-/-} mice were infected with YM and found comparable low levels of IFN-α, IFN-β, IL-6, and IFN-γ in serum compared with those in WT mice (FIG. 20d). Whereas WT mice treated with exogenous recombinant IFN-α/β became resistant to YM infection, Ifnar^{-/-} mice with exogenous recombinant IFN-α/β failed to show reduced parasitemia and improved survival (Extended Data FIG. 20e), indicating that IFNAR is essential for the protective immunity induced by IFN-α/β. To further examine the role of the downstream signaling molecules such as Jak1 and Stat1, Jak1 or Stat1 expression was first knocked down in WT mice by Jak1- or Stat1-specific siRNA at 24 h before infection (FIG. 20e), followed by YM infection at day 0, which were then treated with exogenous recombinant IFN-α/β at 18 h after infection. As expected, WT mice treated with scrambled siRNA plus IFN-α/β were able to inhibit parasitemia and were resistant to YM infection. By contrast, WT mice treated with either Jak1- or Stat1-specific siRNA plus IFN-α/β failed to reduce parasitemia and died (FIG. 16f). These results clearly suggest that both IFNAR and the Jak1/Stat signaling pathway are required for IFN-α/β-mediated protective immunity against YM infection.

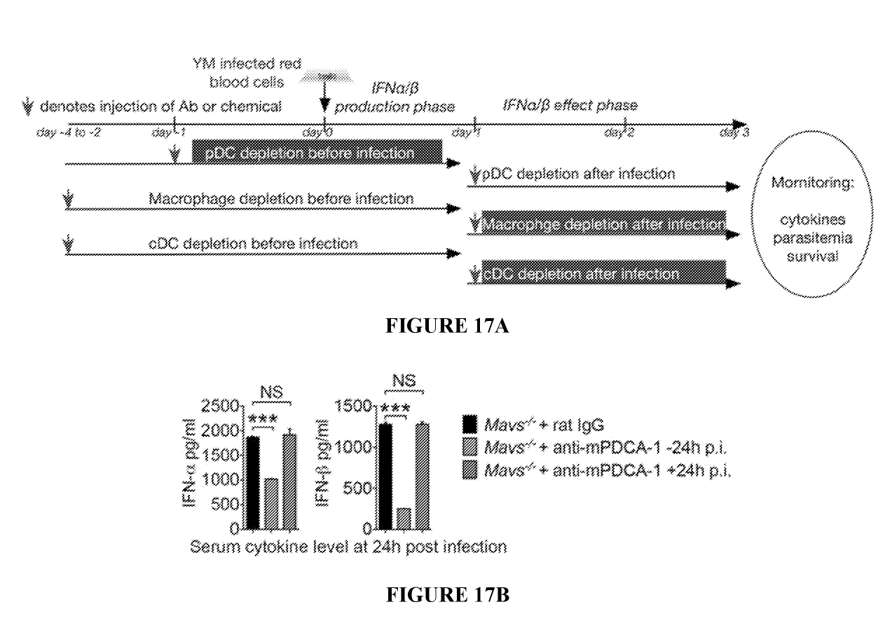

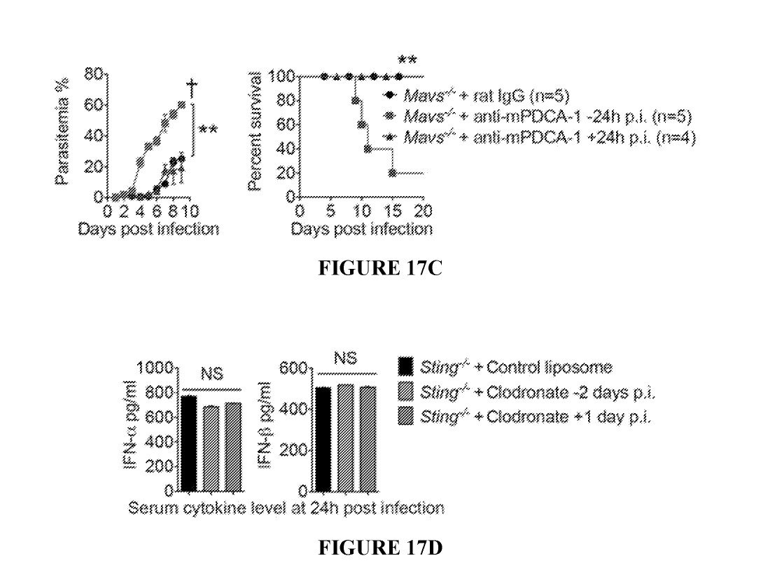

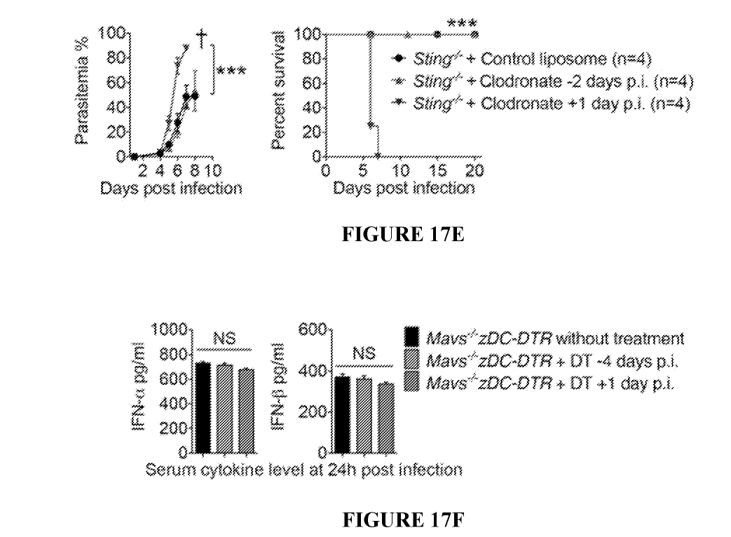

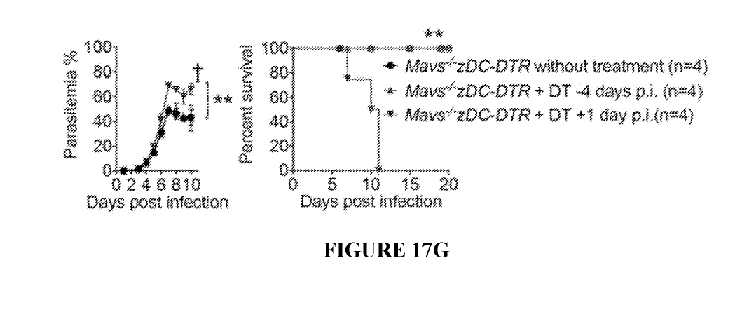

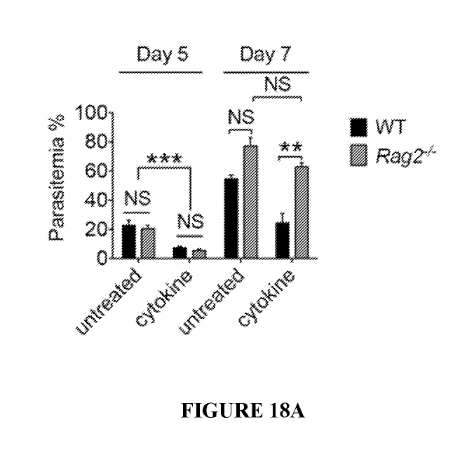

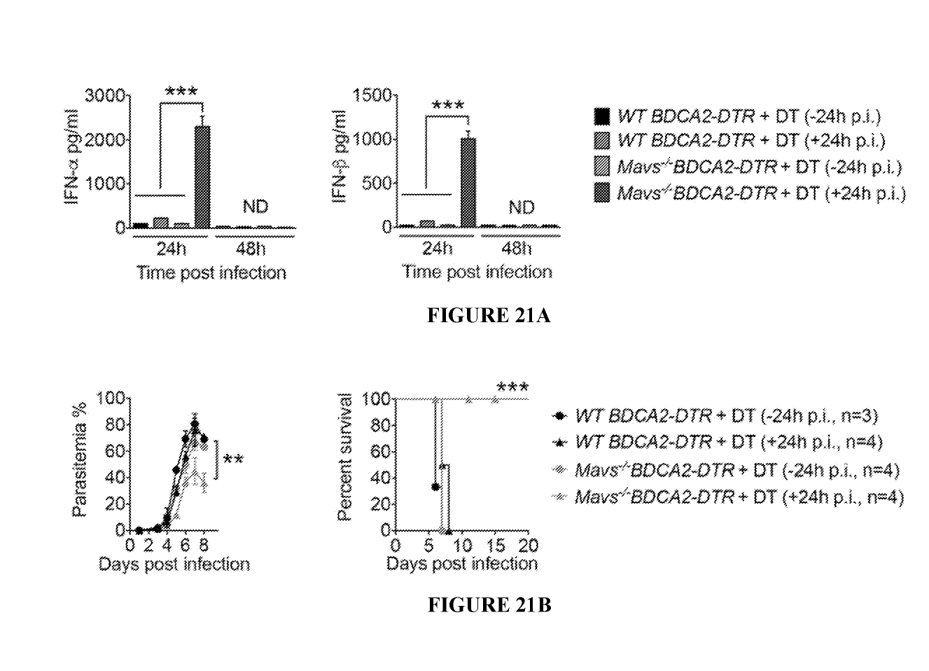

These results showed that the robust production of IFN-α/β in the first 24 h post infection in KO mice (Example 1) and early administration of recombinant IFN-α/β at 18 h, but not at 36 or 48 h post infection, are necessary and sufficient to induce protective immunity through IFNAR and the Jak1/Stat signaling pathway. pDCs are essential for IFN-α/β production, but it is not clear whether other immune cell populations, such as macrophages and conventional DCs (cDCs), are required for inducing both innate and adaptive immunity against YM infection. To determine the relative contribution of innate immune cells, such as pDCs, macrophages, and cDCs, in the IFN-α/β production phase (before and within the first 24 h of infection) or effector phase (after 24 h of infection), specific cell populations were depleted in different phases, and then the effects of specific cell depletion on cytokine production, parasitemia, and host survival was determined (FIG. 17a). We found again that depletion of pDCs with anti-mPDCA-1 in Mavs^{-/-} mice during the cytokine production phase (24 h before infection) markedly reduced IFN-α and IFN-β production; however, depletion of pDCs after 24 of infection had no effect on IFN-α and IFN-β production, compared with mice treated with a control antibody (FIG. 17b). Consistently, Mavs^{-/-} mice treated with anti-mPDCA-1 during the cytokine production phase had increased parasitemia and host mortality. By contrast, Mavs^{-/-} mice treated with anti-mPDCA-1 in the effector phase (at 24 h after infection) had no effect on parasitemia and host survival, compared with control antibody-treated mice (FIG. 17c). To further substantiate these findings, the pDC cell population was depleted in WT BDCA2-DTR and Mavs^{-/-} BDCA2-DTR mice at 24 h before infection or 24 h after infection. As expected, WT BDCA2-DTR mice produced low amounts of IFN-α and IFN-β regardless of pDC depletion at 24 before or after infection (FIG. 21a). By contrast, pDC depletion in Mavs^{-/-}:BDCA2-DTR mice at 24 h before infection abolished the production of serum IFN-α and IFN-β, increased parasitemia and host mortality, compared with pDC depletion at 24 h after infection (FIG. 21a-21b). Collectively, these results clearly demonstrate the importance of pDCs in the production of IFN-α/β within the first 24 h, but not after 24 h after infection, for the protective immune response against YM infection.

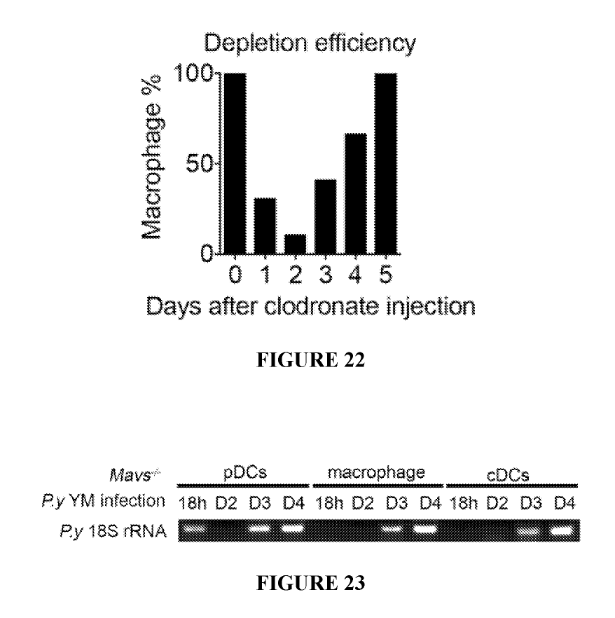

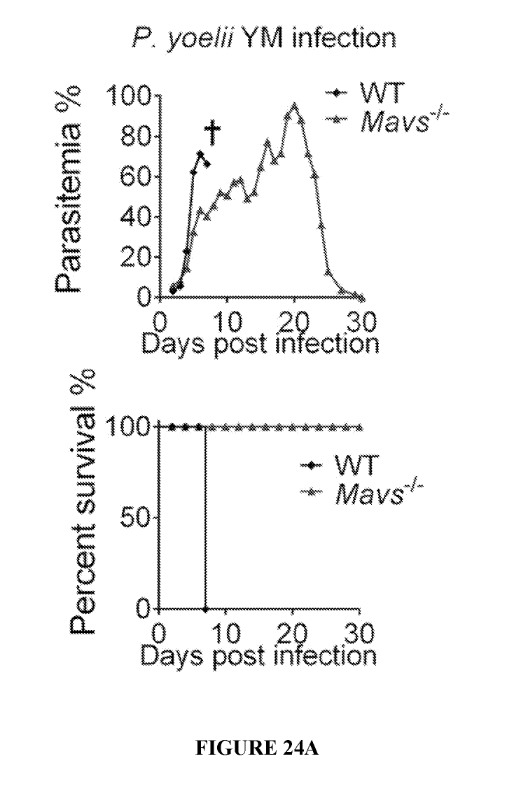

Next, the importance of macrophages in cytokine production, parasitemia, and host mortality was determined by depleting macrophages with clodronate at two days (−2) before or one day after YM infection based on the efficiency and kinetics of macrophage depletion (FIG. 22). Macrophage depletion in Sting^{-/-} mice did not affect serum levels of IFN-α and IFN-β, regardless of depletion at two days before or one day after YM infection (FIG. 17d). However, macrophage depletion during the effector phase (at one day post infection) markedly increased parasitemia and host mortality, compared with a liposome control or treatment at two days before infection (FIG. 17e), suggesting a critical role of macrophages during the effector phase (i.e. after IFN-α and IFN-β production), but not during the cytokine production phase. To determine the relative role of cDCs in anti-malaria immunity, Mavs^{-/-}:zDC-DTR (zinc figure transcription factor-driven DTR expression in cDCs) mice were generated by crossing Mavs^{-/-} mice with zDC-DTR transgenic mice. A previous study showed that DT injection into zDC-DTR bone marrow chimeras results in specific depletion of cDCs in 12 h and maintains cDC depletion for 5 days (Meredith, M. M. et al. J Exp Med 209, 1153-1165 (2012)). Thus DT was injected into Mavs^{-/-}:zDC-DTR chimeric mice at four days (−4) before or at one day post infection. There was no change in the serum levels of IFN-α and IFN-β when DT was injected either before or after YM infection (FIG. 17f), suggesting that cDCs are not responsible for the IFN-α and IFN-β production during the early phase (24 h) of infection. However, Mavs^{-/-}:zDC-DTR mice treated with DT after 24 h of infection showed increased parasitemia and died sooner after infection compared with untreated or treated with DT at four days before infection (FIG. 17g), suggesting that cDCs are required for generating protective immunity during the effector phase (after 24 h after infection). Consistent with these results, malaria 18S rRNA could be detected in pDCs, but not in macrophages or cDCs, at 18 h post infection. However, malaria 18S rRNA could be detected in macrophages and cDCs of Mavs^{-/-} mice at day 3 post infection (FIG. 23), suggesting that pDCs are major cell population that detect P. yoelii YM infection within the first 24 h, whereas macrophages and cDCs are involved in the detection of YM infection at later time points. Taken together, these results suggest that pDCs, but not macrophages or cDCs, are critically required for the production of IFN-α and IFN-β in the first 24 h after YM infection (cytokine production phase), whereas macrophages and cDCs are required for IFN-α/β-induced immunity against YM after 24 h post infection (effector phase).

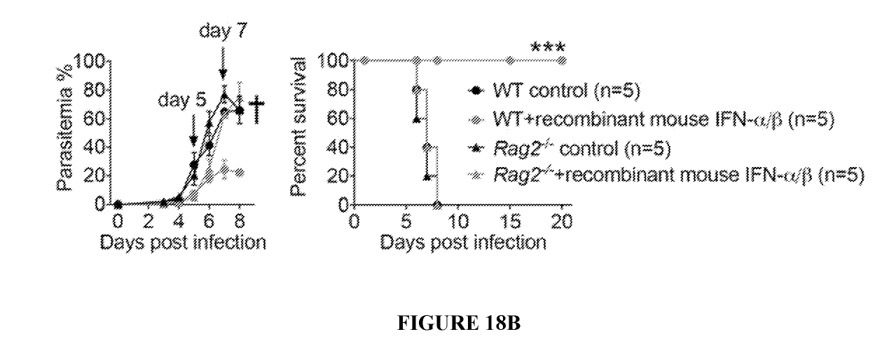

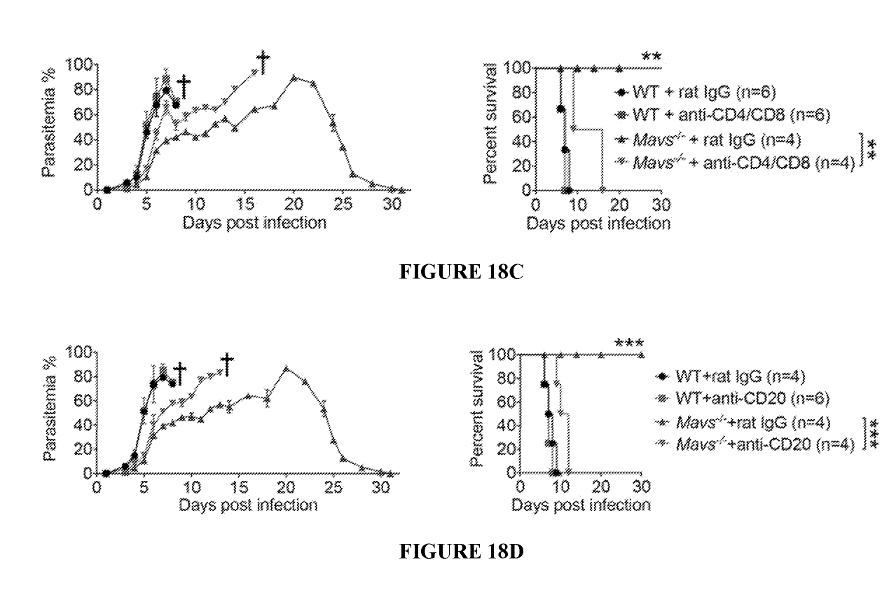

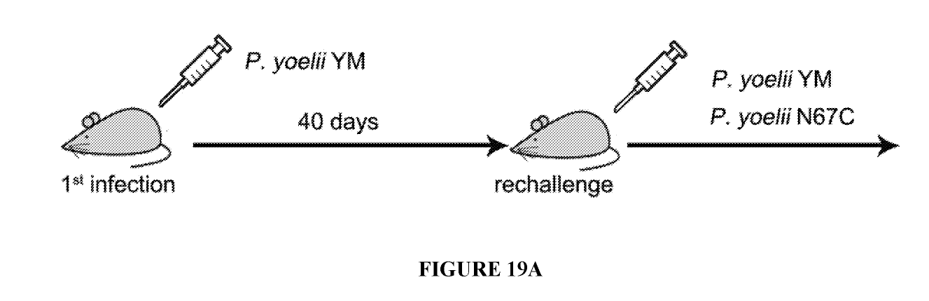

Next, experiments were conducted to determine whether B and T cells are required for IFN-α/β-induced immunity against YM, in particular for clearing malaria in the late phase of infection. To test this possibility, WT and Rag2-deficient mice, which lack B and T cells, were infected with YM in the presence or absence of exogenous murine recombinant IFN-α and IFN-β. Although cytokine-treated WT and Rag2^{-/-} mice demonstrated reduced parasitemia compared with untreated WT and Rag2^{-/-} mice, no differences in the percent of parasitemia between WT and Rag2^{-/-} mice was found, either in untreated groups or cytokine-treated groups in the first 5 days after infection (FIG. 18a). However, after 7 days post infection, parasitemia in cytokine-treated Rag2^{-/-} mice markedly increased to the levels observed in untreated WT or untreated Rag2^{-/-} mice (FIG. 18a-18b). WT untreated, Rag2^{-/-} untreated, and Rag2^{-/-} cytokine-treated mice died within 8 days after YM infection, whereas cytokine-treated WT mice maintained significantly lower parasitemia than the other three groups and survived beyond 8 days (FIG. 18b). To further support the role of T cells, T cell depletion was performed in Mavs^{-/-} mice using anti-CD4 and anti-CD8 antibodies. WT mice rapidly increased parasitemia and died within 8 days after YM infection, regardless of control or anti-CD4/anti-CD8 treatment (FIG. 18c). Consistent with results from Rag2^{-/-} mice, Mavs^{-/-} mice treated with anti-CD4 and anti-CD8 antibodies showed markedly increased parasitemia after 7 days post infection and all mice died within 16 days after infection. Conversely, Mavs^{-/-} mice treated with control antibody maintained low parasitemia and survived (FIG. 18c). Similarly, B cells were also important for protective immunity and eventually clearing parasites at later time point by depleting B cell in Mavs^{-/-} mice using anti-CD20 antibody (FIG. 18d). Overall, these results suggest that B and T cells are required for generating adaptive immunity to control parasitemia at 6 days after infection and to clear parasites in 3-4 weeks.

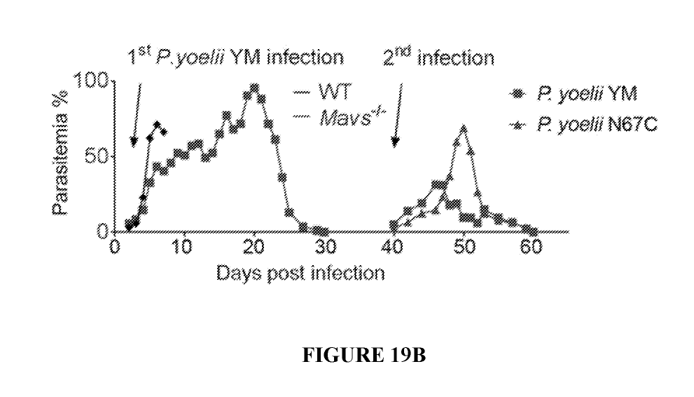

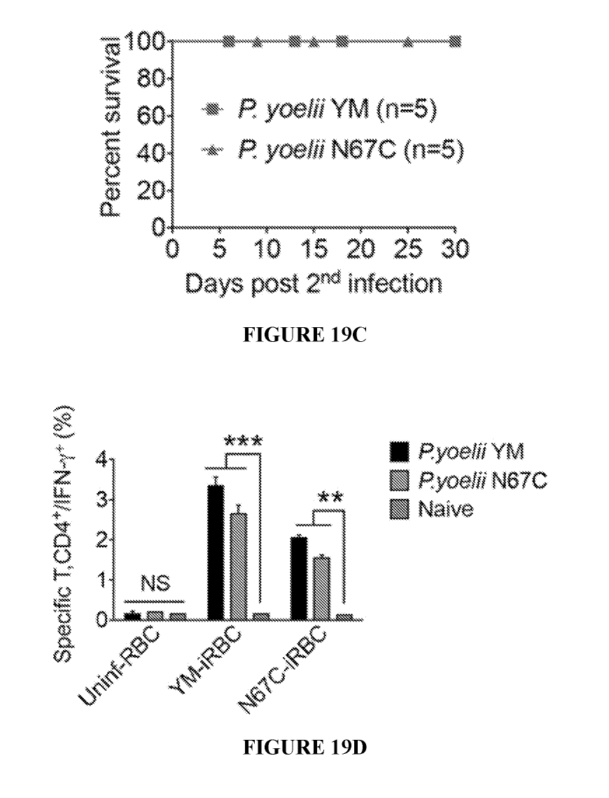

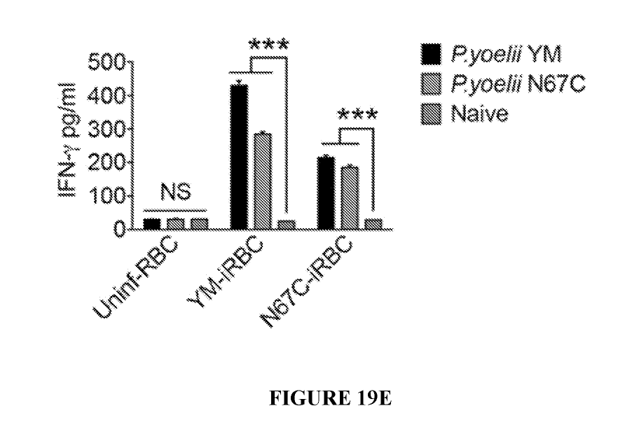

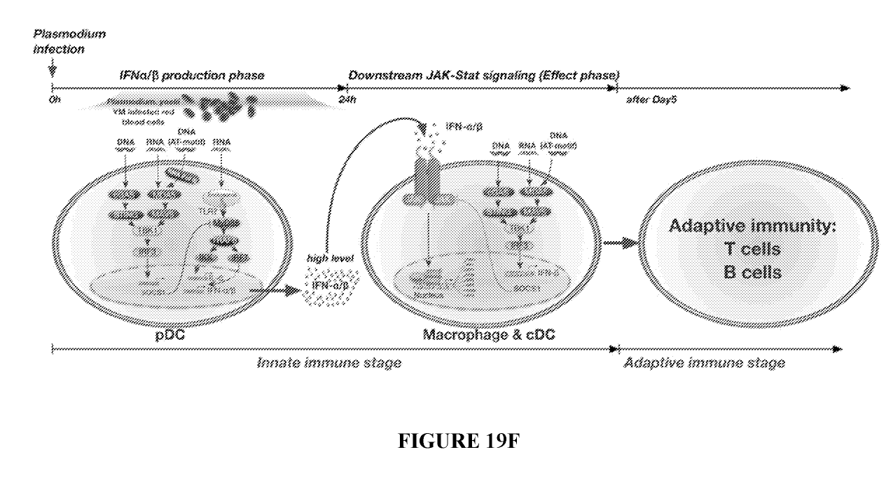

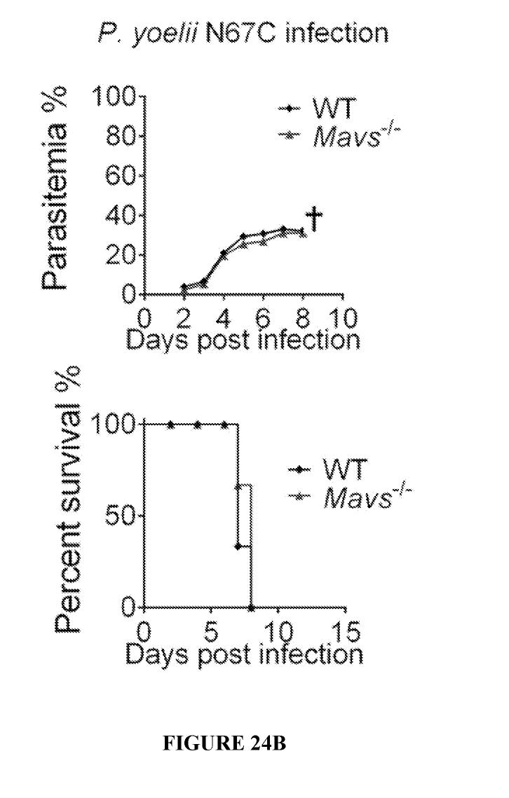

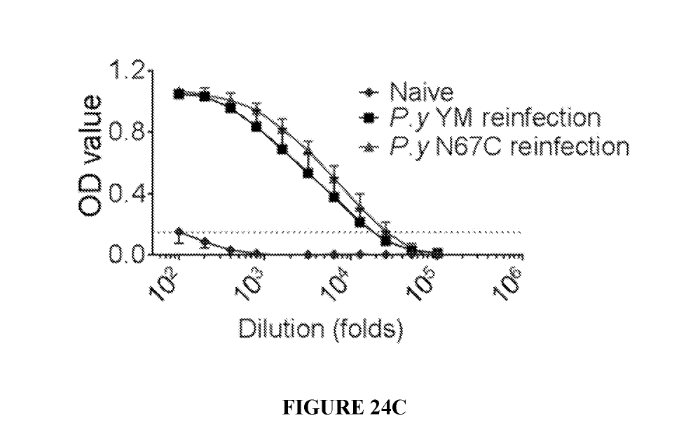

Next, experiments were conducted to determine whether innate and adaptive immunity developed in Mavs^{-/-}, mice after YM infection could generate adaptive and memory immune responses for the second challenge of the same (P. yoelii YM) or different (lethal P. yoelii N67C) strain, even though the pathogenesis of these two strains are different. It was reasoned that adaptive immunity generated by P. yoelii YM may respond to those antigens shared by P. yoelii YM and P. yoelii N67C. To test this possibility, WT and Mavs^{-/-} mice were first infected with P. yoelii YM, then the same YM strain or different lethal strain P. yoelii N67C as re-challenged with at 40 days after the first infection (FIG. 19a). Parasitemias of Mavs^{-/-} mice after the second YM infection was much lower than the first infection (FIG. 19b, 24a). Furthermore, Mavs^{-/-} mice initially infected with YM became resistant to lethal P. yoelii N67C strain and cleared the parasites during the second challenge (FIG. 19b-19c), although previously uninfected Mavs^{-/-} mice were sensitive to lethal P. yoelii N67C infection (FIG. 24b). These results indicate that Mavs^{-/-} mice generated potent adaptive immune responses after an initial infection with YM, and thus become resistant to the second challenge with the same or different strains.

To further understand cellular and molecular basis of YM-induced adaptive immunity in Mavs^{-/-} mice, splenocytes were isolated from YM-infected Mavs^{-/-} mice at day 60 after the initial infection (the time that mice had cleared the parasites after the second infection), and then stimulated with crude antigens (iRBC, infected red blood cells) from different malaria strains. The percentage of splenic IFN-γ⁺ CD4⁺ T cells from immunized Mavs^{-/-} mice were much higher than splenic IFN-γ⁺ CD4⁺ T cells from naïve Mavs^{-/-} mice (FIG. 19d). Consistently, IFN-γ protein levels and concentrations of IgG immunoglobulin in serum were significantly higher than in naïve Mavs^{-/-} mice (FIG. 19e, 24c), suggesting that YM-infected Mavs^{-/-} mice developed potent adaptive immune responses to antigens potentially shared by YM and N67C strains.

This study has identified the role, mechanism, and sequential order of immune responses induced by IFN-α/β. These results clearly demonstrate the requirement for pDCs with deficient in genes (Mavs, Mda5, and Sting) involved in IRF3-dependent type I IFN signaling for robust production of IFN-α and IFN-β through MyD88-dependent type I IFN signaling pathway during the first 24 h of infection, but not required after the cytokine production phase. By contrast, depletion of macrophages or cDCs at 24 h post infection (effector phase) resulted in increased parasitemia and host mortality, suggesting that these cell types participated in anti-malaria immunity, which is consistent with previous results showing that classic DCs are required for inducing T cell responses (Voisine, C., et al. Int J Parasatol 40:711-719005 (2010)). Although B cells and T cells are not required for IFN-α and IFN-β production and parasitemia control during the first 5-6 days of YM infection, they are required for the control of parasitemia, host mortality and clearance of parasites at later times (>6 days), as suggested by previous studies using cerebral malaria (Haque, A. et al. J Clin Invest 124:2483-2496 (2014); Ball, E. A. et al. J Immunol 190:5118-5127 (2013)). However, previous studies show that recombinant human IFN-α or murine IFN-β treatment protect mice from ANKA infection (Vigario, A. M. et al. J Immunol 178:6416-6425 (2007); Morrell, C. N. et al. Infect Immun 79:1750-1758 (2011)), but these findings contradict with results using mice deficient in Sting, Tbk1, Irf3/Irf7, or Ifnar expression, which are resistant to Plasmodium berghei ANKA infection (Haque, A. et al. J Clin Invest 124:2483-2496 (2014); Sharma, S. et al. Immunity 35:194-207 (2011)). P. berghei ANKA induces type I IFN cytokine production (peaked) at day 4 after infection (Haque, A. et al. J Clin Invest 124:2483-2496 (2014)), but YM infection induces type I IFN cytokine (peaked) at 24 h (day 1) in this study. Importantly, type I IFN cytokines produced at day 4 during P. berghei ANKA inhibit, rather than enhance, T cell immune response through IFNAR-mediated downstream type I IFN signaling (Haque, A. et al. J Clin Invest 124:2483-2496 (2014); Haque, A. et al. Eur J Immunol 41:2688-2698 (2011)). Similarly, there was a higher serum level of IFN-α/β in Mavs^{-/-} mice than WT mice at 24 h after P. yoelii N67C, but they were still sensitive to P. yoelii N67C infection. Thus, it is clear that type I IFN cytokines play a critical role in regulating immune response and dictating host mortality; however, the opposing effects (i.e., the protective or detrimental) of type I IFN cytokines on host mortality may be determined by multiples factors, including the timing and level of type I IFN production during infection of different malaria strains, cell tropism (targeting different cell types), immune trafficking to infected tissues, as well as their distinct pathogeneses (systemic versus organ-specific pathological diseases) (Wu, J. et al. Proc Natl Acad Sci USA 111:E511-520 (2014); Gun, S. Y., et al. Mediators of inflammation 2014:243713 (2014)). Using YM as a model (FIG. 19f), a vaccine strategy was developed to generate potent innate and adaptive immunity using KO (Mavs^{-/-}) mice that can effectively protect the host from the second challenge of the same or different lethal malaria strains: 1) lethal YM infection activates MyD88-dependent type I IFN signaling pathway for robust production of IFN-α and IFN-β in pDCs in Mavs-deficient mice in the first 24 h post infection; 2) IFN-α and IFN-β then activate downstream signaling pathways via IFNAR/Stat1/Jak1 in other innate immune cells, such as macrophages and cDCs, for priming potent adaptive immunity mediated by B and T cells to clear YM infection at later times; 3) importantly, adaptive immunity generated in YM-infected Mavs-deficient mice can markedly inhibit the second infection of the same or even different (such as P. yoelii N67C) strains, and eventually clear the parasites in 3-4 weeks. Thus, these findings not only identify a previously unrecognized critical role of stage-dependent type I IFN production and its downstream signaling pathways and specific contribution of different immune cells for developing innate and adaptive immunity, but also provide new strategies for the development of therapeutic malaria vaccines.

Methods

Malaria Parasites and Mice.

The parasite Plasmodium yoelii YM and Plasmodium yoelii N67C have been previously described (Wu, J. et al. Proc Natl Acad Sci USA 111:E511-520 (2014); Li, J. et al. Proc Natl Acad Sci USA 108:E374-382 (2011)). For plasmodium infection, 0.2 (low) or 2×10⁶ iRBCs (otherwise, indicated specifically in the figure legend) suspended in 200 μl PBS from the donor mice were intraperitoneally injected into experimental mice. Parasitemias were monitored daily by examination of Giemsa-stained thin tail blood smears. Female mice C57BL/6 (WT), Mavs^{-/-}, Mda5^{-/-}, Sting^{-/-}, BDCA2-DTR, zDC-DTR, Ifnar^{-/-}, and Rag2^{-/-} mice were purchased from The Jackson Laboratory. All mouse-related procedures were performed according to experimental protocols approved by the Animal Care and Welfare Committee at Houston Methodist Research Institute and in accordance with NIH-approved animal study protocol LMVR-11E.

In Vivo Receptor Blockade, Cytokine Treatment, and Cell Depletion.

To block type I IFN receptor, anti-mouse interferon α/β receptor antibodies (Leinco Technologies), in the amount of 500 μg in PBS at day 0, 2, 4, and 250 μg at day 6 after infection (or indicated specifically in the figure legend), were intraperitoneally injected into WT and deficient mice. Mouse IgG antibody was used as control, and parasitemias were monitored by daily Giemsa-stained blood smears. To test the role of type I IFN cytokines, C57BL/6 mice were first infected with P. yoelii YM, and then intravenously injected with 800 U/g mouse recombinant IFN-α and IFN-β, recombinant IL-6, or recombinant IFN-γ at the indicated time points. Bovine serum albumin (BSA, 0.1%) in PBS served as a control, and parasitemias were monitored by daily Giemsa-stained blood smears. To deplete pDCs, pDC-depleting functional-grade mAb (anti-mPDCA-1 IgG, clone JF05-1C2.4.1) was purchased from Miltenyi Biotec (Auburn, Calif.), and the corresponding isotype control IgG served as control. Two intraperitoneally injections of antibody (250 μg/mouse) per mouse were administered 12 h prior and after plasmodium YM infection or indicated specifically in the figure legend. To deplete macrophage, clodronate liposomes (from Dr. Nico. Van Rooijen) were injected intraperitoneally at 750 μg/injection at the indicated times, control liposomes served as control. T cells were depleted by intraperitoneally injection of anti-CD4 and anti-CD8 antibody every 3 days from 1 day before infection. B cells were depleted by intraperitoneally injection of anti-CD20 antibody every 4 days from 7 days before infection.

Flow Cytometry and ELISA.

Mouse splenocytes were prepared from spleens cut into small fragments and digested with collagenase D (1 mg/ml, Sigma) and DNAse I (20 μg/ml, Sigma) in RPMI-1640 medium for 30 min. Single cells were collected and cultured for 5 h in the presence of Golgi Stop solution (BD Biosciences), harvested for the surface molecular staining with the appropriate fluorescent mAbs (anti-CD4 and anti-CD8), and then fixed, permeabilized (FIX AND PERM, Invitrogen) and stained with fluorescein-labeled cytokine-specific mAbs (PE-anti-mouse IFN-γ was purchased from eBioscience) following the manufacturer's instructions. Appropriate fluorescein-conjugated, isotype-matched mAbs were used as negative controls. Cells were analyzed using the BD FACS Aria II. For ELISA, mouse serum or cell supernatants were collected at the indicated times after infection or stimulation and subjected to analysis with commercial ELISA kits for mouse IFN-α, IFN-β, (PBL Biomedical Laboratories) or IFN-γ, IL-6 (eBioscience), or IgG (SouthernBiotech) following the manufacturer's instructions.

RNAi-Mediated Knockdown in Mice.

In Vivo Ready siRNAs were mixed with Invivofectamine 3.0 liposomes (Invitrogen) following the manufacturer's instructions and injected intravenously in a volume of 100 μl at a dose of 5 mg/kg. Mice were infected with P. yoelii YM (0.5×10⁶ iRBCs) 48 h after siRNA treatment.

RNA Preparation and qPCR.

Total RNA was harvested from splenic tissue or stimulated cells using the TRIzol reagent (Invitrogen) and the complimentary cDNA was generated using reverse transcriptase III (Invitrogen). Real-time PCR was carried out using the ABI Prism 7000 analyzer (Applied Biosystems), using the SYBR GreenER qPCR Super Mix Universal (Invitrogen) and specific primers.

Diphtheria Toxin (DT) Treatment.

Deficient (Mda5^{-/-}, Mavs^{-/-}, and Sting^{-/-}) mice were crossed with BDCA2-DTR transgenic mice and treated with diphtheria toxin (DT, Sigma-Aldrich) intraperitoneally (i.p.) at dose of 100-120 ng/mouse. pDCs were depleted by DT injection at 1 day before and 1, 3, and 5 days after P. yoelii YM infection. For cDC depletion, bone marrow chimeras were reconstituted for at least 6 to 8 weeks after lethal irradiation (950 cGy) and i.v. transferred with 10×10⁶ bone marrow cells from Mavs^{-/-}:zDC-DTR mice, then injected with DT at a dose of 2.5 ng per gram of body weight.

Statistical Analysis.

All analyses were performed using GraphPad Prism version 5.0 (GraphPad Software, La Jolla, Calif.). Data are presented as means±s.d., unless otherwise stated. Statistical significance of differences between two groups was assessed by unpaired Student t tests and a p value of <0.05 was considered significant.

### Example 3: Mice Deficient in MAVS are Sensitive to Lethal P. yoelii N67C or P. berghei ANKA Infection

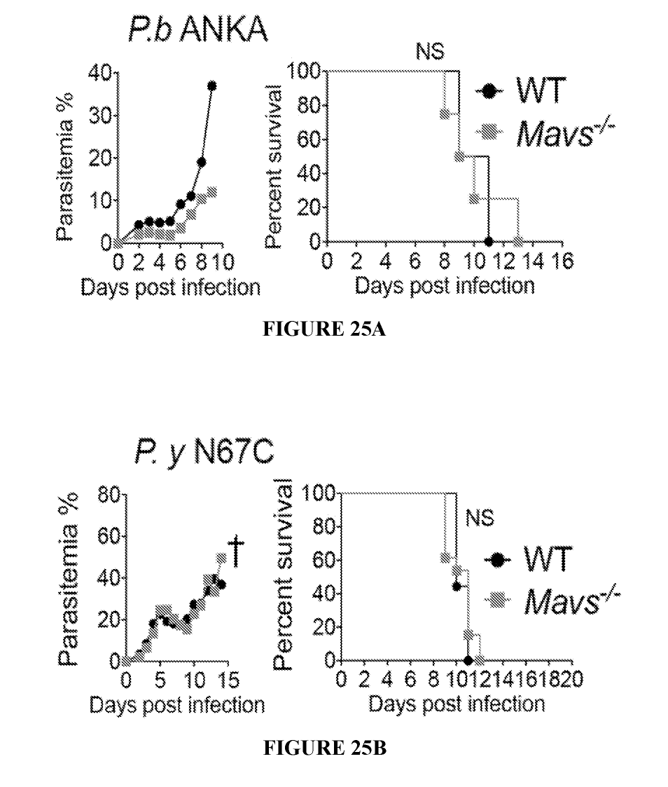

To test the generality of mice resistant to malaria infection, Mavs^{-/-} mice were infected with lethal malaria strains P. yoelii N67C and P. berghei ANKA. Mavs^{-/-} mice showed similar trend of parasitemia and morality as WT mice after P. yoelii N67C and P. berghei ANKA infection and eventually died at about day 10 after infection (FIG. 25). This phenomenon is quite different from that after P. yoelii YM infection, which suggest the unique feature of type I IFN in YM infection or function of type I IFN signaling could be both protective or pathogenic after different malaria strain infection.

### Example 4: Different Cytokine Patterns after Lethal P. yoelii YM, P. yoelii N67C or P. berghei ANKA Infection

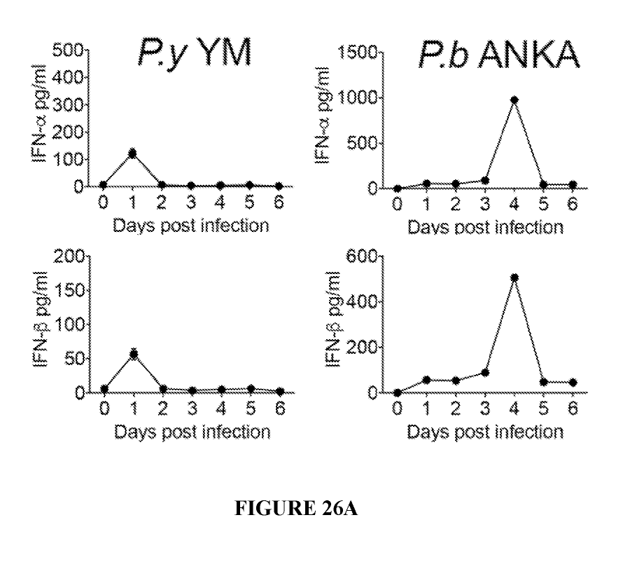

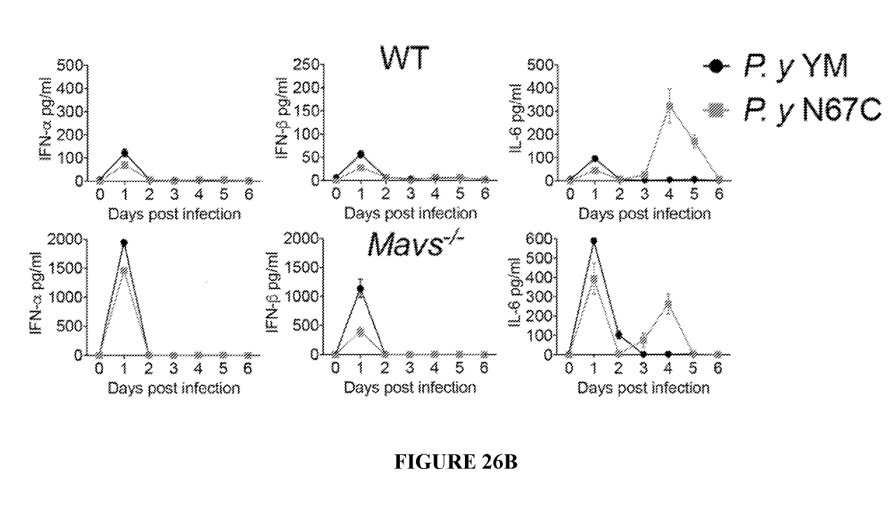

To understand why type I IFN functions differently in host anti-pathogen response after infection, different lethal Plasmodium strains were used to investigate the strain specific/shared immune responses and pathogenesis by infecting C57BL/6 (WT) mice with P. yoelii YM or P. berghei ANKA, and then serum cytokine levels were determined. WT mice produced low amounts of type I IFN at day 1 (24 h) after YM infection. By contrast, ANKA-infected WT mice did not produce any IFN-a/b at day 1, but produced large amounts of IFN-a/b at day 4 (peak) after infection (FIG. 26A), indicating that the delayed IFN-a/b at day 4 post infection inhibits immune response. Furthermore, similar experiments were performed using WT and Mavs^{-/-} mice infected with YM and N67C. WT mice produced low amounts of IFN-a/b and IL-6 at day 1 post both YM and N67C infection, but Mavs^{-/-} mice produced large amounts of IFN-a/b and IL-6 than WT mice at day 1 after YM or N67C infection (FIG. 26B). However, a second peak of IL-6 production was detected at day 4 in N67C-infected WT and Mavs^{-/-} mice, but not in YM-infected mice (FIG. 26B). It was reasoned that the second peak production of IL-6 at day 4 might impair the host immune responses, resulting in the death of N67C-infected Mavs^{-/-} mice.

### Example 5: Blockade of Late Cytokine (IFN-α/b or IL-6) Signaling Protects Mavs^{-/-} Mice from Lethal Malaria Infection

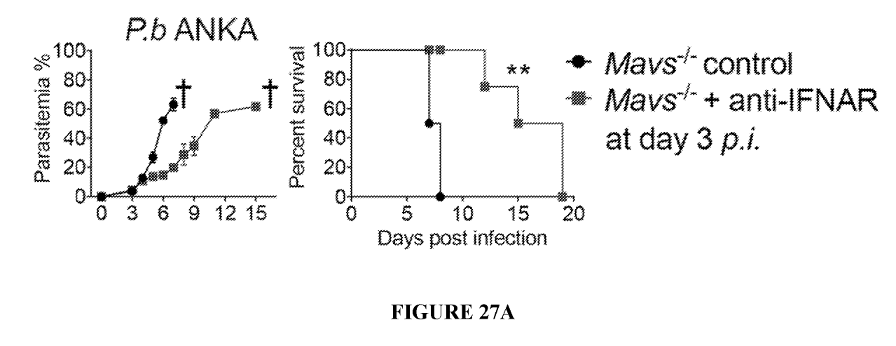

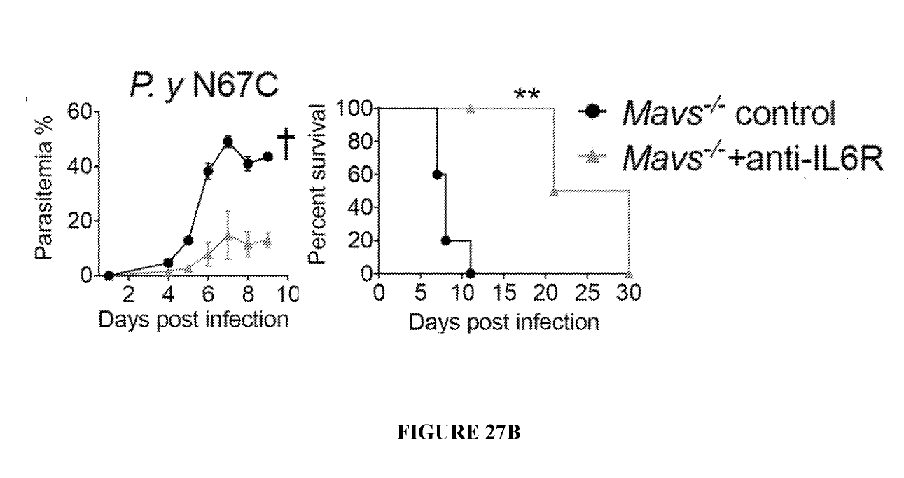

To determine whether the delayed production of IL-6 or type I IFN at day 4 after infection dampens early type I IFN-induced immune responses, IL-6 signaling was blocked with an anti-IL-6R antibody at day 3 post N67C or IFN-a/b signaling with anti-IFNAR at day 3 post ANKA infection. Anti-IL-6R antibody treatment could significantly prolong Mavs^{-/-} mouse survival after N67C infection (FIG. 27). Similar results were obtained with anti-IFNAR antibody treatment in mice with ANKA infection (FIG. 27). These results support the hypothesis that the delayed production of IL-6 or IFN-a/b after N67C or ANKA infection inhibits host immune response.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A composition comprising;
a TLR9 or TLR7 ligand and a SOCS1 pathway antagonist in a pharmaceutically acceptable carrier.

2. The composition of claim 1, wherein the SOCS1 pathway antagonist comprises a SOCS1 inhibitor.

3. The composition of claim 2, wherein the SOCS1 inhibitor comprises a gene silencing functional nucleic acid.

4. The composition of claim 3, wherein the gene silencing functional nucleic acid comprises an RNAi or siRNA.

5. The composition of claim 1, wherein the SOCS1 pathway antagonist comprises a TBK1 inhibitor.

6. The composition of claim 1, wherein the SOCS1 pathway antagonist comprises an IRF3 inhibitor.

7. The composition of claim 1, wherein the TLR9 or TLR7 ligand comprises a CpG oligonucleotide.

8. The composition of claim 1, further comprising a pathogen.

9. The composition of claim 8, wherein the pathogen comprises a Plasmodium.

10. The composition of claim 9, wherein the Plasmodium is live, attenuated.

11. The composition of claim 1, further comprising interferon-α, interferon-β, or a combination thereof.

12. The composition of claim 1, further comprising an adjuvant.

13. A method for vaccinating a subject for a pathogen, comprising administering to a subject in need thereof the composition of claim 1.

14. A method for vaccinating a subject for cancer, comprising administering to a subject in need thereof the composition of claim 1 and a tumor antigen.

15. The method of claim 14, wherein the composition comprises the tumor antigen.

16. The method of claim 14, wherein the composition and the tumor antigen are administered separately.

17. The method of claim 14, further comprising administering to the subject a composition comprising a checkpoint inhibitor.

18. The method of claim 14, further comprising administering to the subject a composition comprising interleukin-2, interferon-α, or combination thereof.

19. A method for enhancing tumor immunity in a subject, comprising administering to a subject diagnosed with a tumor the composition of claim 1.

20. The method of claim 19, further comprising administering to the subject a composition comprising a tumor antigen.

21. The method of claim 19, further comprising administering to the subject a composition comprising a checkpoint inhibitor.

22. The method of claim 21, further comprising administering to the subject a composition comprising interleukin.
